(19) United States
(12) Patent Application Publication
(10) Pub. No.: US 2010/0068204 A1
(43) Pub. Date: Mar. 18, 2010

(54) 4-ARYLOXYQUINOLIN-2(1H)-ONES AS MTOR KINASE AND PI3 KINASE INHIBITORS, FOR USE AS ANTI-CANCER AGENTS

(75) Inventors: Hwei-Ru Tsou, New City, NY (US); Gary Harold Birnberg, Tuxedo Park, NY (US); Gloria Jean MacEwan, Monroe, NY (US); Mercy Adufa Otteng, Pearl River, NY (US); Pawel Wojciech Nowak, Woodcliff Lake, NJ (US); Semiramis Ayral-Kaloustian, Tarrytown, NY (US); Tarek Suhayl Mansour, New City, NY (US); Arie Zask, New York, NY (US); Ker Yu, Pine Brook, NJ (US)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/558,661

(22) Filed: Sep. 14, 2009

Related U.S. Application Data
(60) Provisional application No. 61/096,420, filed on Sep. 12, 2008.

(51) Int. Cl. A61K 39/395; C07D 215/00; A61K 31/47

(52) U.S. Cl. 424/133.1; 546/155; 514/312

(57) ABSTRACT

Compounds of the formula I

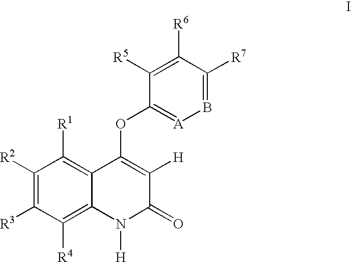

and pharmaceutically acceptable salts thereof, wherein A, B, R¹, R², R³, R⁴, R⁵, R⁶, and R⁷ are defined as set forth herein are disclosed. Also disclosed are pharmaceutical compositions comprising the compounds of the invention and a pharmaceutically acceptable carrier, methods of making the compounds of the invention and methods of using the compounds for inhibiting mTOR and PI3 kinases and for treating cancers.

### FIELD OF THE INVENTION

The invention relates to 4-ARYLOXYQUINOLIN-2(1H)-ONE compounds, compositions comprising a compound of the present invention, methods of synthesizing compounds of the present invention, and methods for treating mTOR-related diseases comprising the administration of an effective amount of a compound of the present invention. The invention also relates to methods for treating PI3K-related diseases comprising the administration of an effective amount of a compound of the present invention.

### BACKGROUND OF THE INVENTION

Phosphatidylinositol (hereinafter abbreviated as “PI”) is one of the phospholipids in cell membranes. In recent years it has become clear that PI plays an important role also in intracellular signal transduction. It is well recognized in the art that PI (4,5) bisphosphate (PI(4,5)P2 or PIP2) is degraded into diacylglycerol and inositol (1,4,5) triphosphate by phospholipase C to induce activation of protein kinase C and intracellular calcium mobilization, respectively [M. J. Berridge et al., Nature, 312, 315 (1984); Y. Nishizuka, Science, 225, 1365 (1984)].

In the late 1980s, phosphatidylinositol-3 kinase (“PI3K”) was found to be an enzyme that phosphorylates the 3-position of the inositol ring of phosphatidylinositol [D. Whitman et al., Nature, 332, 664 (1988)]. When PI3K was discovered, it was originally considered to be a single enzyme. Recently however, it was clarified that a plurality of PI3K subtypes exists. Three major subtypes of PI3Ks have now been identified on the basis of their in vitro substrate specificity, and these three are designated class I (a & b), class II, and class III [B. Vanhaesebroeck, Trend in Biol. Sci., 22, 267 (1997)].

The class Ia PI3K subtype has been most extensively investigated to date. Within the class Ia subtype there are three isoforms (α, β, & δ) that exist as hetero dimers of a catalytic 110-kDa subunit and regulatory subunits of 50-85 kDa. The regulatory subunits contain SH2 domains that bind to phosphorylated tyrosine residues within growth factor receptors or adaptor molecules and thereby localize PI3K to the inner cell membrane. At the inner cell membrane PI3K converts PIP2 to PIP3 (phosphatidylinositol-3,4,5-trisphosphate) that serves to localize the downstream effectors PDK1 and Akt to the inner cell membrane where Akt activation occurs. Activated Akt mediates a diverse array of effects including inhibition of apoptosis, cell cycle progression, response to insulin signaling, and cell proliferation. Class la PI3K subtypes also contain Ras binding domains (RBD) that allow association with activated Ras providing another mechanism for PI3K membrane localization. Activated, oncogenic forms of growth factor receptors, Ras, and even PI3K kinase have been shown to aberrantly elevate signaling in the PI3K/Akt/mTOR pathway resulting in cell transformation. As a central component of the PI3K/Akt/mTOR signaling pathway PI3K (particularly the class Ia a isoform) has become a major therapeutic target in cancer drug discovery.

Substrates for class 1 PI3Ks are PI, PI(4)P and PI(4,5)P2, with PI(4,5)P2 being the most favored. Class I PI3Ks are further divided into two groups, class Ia and class Ib, because of their activation mechanism and associated regulatory subunits. The class Ib PI3K is p110γ that is activated by interaction with G protein-coupled receptors. Interaction between p110γ and G protein-coupled receptors is mediated by regulatory subunits of 110, 87, and 84 kDa.

PI and PI(4)P are the known substrates for class II PI3Ks; PI(4,5)P2 is not a substrate for the enzymes of this class. Class II PI3Ks include PI3K C2α, C2β, and C2γ isoforms, which contain C2 domains at the C terminus, implying that their activity is regulated by calcium ions.

The substrate for class III PI3Ks is PI only. A mechanism for activation of the class III PI3Ks has not been clarified. Because each subtype has its own mechanism for regulating activity, it is likely that activation mechanism(s) depend on stimuli specific to each respective class of PI3K.

The compound PI103 (3-(4-(4-morpholinyl)pyrido[3′,2′:4,5]furo[3,2-d]pyrimidin-2-yl)phenol) inhibits PI3K_{α} and PI3K_{γ} as well as the mTOR complexes with IC₅₀ values of 2, 3, and 50-80 nM respectively. I.P. dosing in mice of this compound in human tumor xenograft models of cancer demonstrated activity against a number of human tumor models, including the glioblastoma (PTEN null U87MG), prostate (PC3), breast (MDA-MB-468 and MDA-MB-435) colon carcinoma (HCT 116); and ovarian carcinoma (SKOV3 and IGROV-1); (Raynaud et al, Pharmacologic Characterization of a Potent Inhibitor of Class I Phosphatidylinositide 3-Kinases, Cancer Res. 2007 67: 5840-5850).

The compound ZSTK474 (2-(2-difluoromethylbenzoimidazol-1-yl)-4,6-dimorpholino-1,3,5-triazine) inhibits PI3K_{α} and PI3K_{γ} but not the mTOR enzymes with IC₅₀ values of 16, 4.6 and >10,000 nM respectively (Dexin Kong and Takao Yamori, ZSTK474 is an ATP-competitive inhibitor of class I phosphatidylinositol 3 kinase isoforms, Cancer Science, 2007, 98:10 1638-1642). Chronic oral administration of ZSTK474 in mouse human xenograft cancer models, completely inhibited growth that originated from a nonsmall-cell lung cancer (A549), a prostate cancer (PC-3), and a colon cancer (WiDr) at a dose of 400 mg/kg. (Yaguchi et al, Antitumor Activity of ZSTK474, a New Phosphatidylinositol 3-Kinase Inhibitor, J. Natl. Cancer Inst. 98: 545-556).

The compound NVP-BEZ-235 (2-methyl-2-(4-(3-methyl-2-oxo-8-(quinolin-3-yl)-2,3-dihydro-1H-imidazo[4,5-c]quinolin-1-yl)phenyl)propanenitrile) inhibits both PI3K_{G}, and PI3K_{γ} as well as the mTOR enzyme with IC₅₀ values 4, 5, and “nanomolar”. Testing in human tumor xenograft models of cancer demonstrated activity against human tumor models of prostrate (PC-3) and glioblastoma (U-87) cancer. It entered clinical trials in December of 2006 (Verheijen, J. C. and Zask, A., Phosphatidylinositol 3-kinase (PI3K) inhibitors as anticancer drugs, Drugs Fut. 2007, 32(6): 537-547).

The compound SF-1126 (a prodrug form of LY-294002, which is 2-(4-morpholinyl)-8-phenyl-4H-1-benzopyran-4-one) is “a pan-PI3K inhibitor”. It is active in preclinical mouse cancer models of prostrate, breast, ovarian, lung, multiple myeloma, and brain cancers. It began clinical trials in April, 2007 for the solid tumors endometrial, renal cell, breast, hormone refractory prostate, and ovarian cancers. (Verheijen, J. C. and Zask, A., Phosphatidylinositol 3-kinase (PI3K) inhibitors as anticancer drugs, Drugs Fut. 2007, 32(6): 537-547).

Exelixis Inc. (So. San Francisco, Calif.) recently filed INDs for XL-147 (a selective pan-PI3K inhibitor of unknown structure) and XL-765 (a mixed inhibitor of mTOR and PI3K of unknown structure) as anticancer agents. TargeGen's short-acting mixed inhibitor of PI3Kγ and δ, TG-100115, is in phase I/II trials for treatment of infarct following myocardial ischemia-reperfusion injury. Cerylid's antithrombotic PI3Kβ inhibitor CBL-1309 (structure unknown) has completed preclinical toxicology studies.

According to Verheijen, J. C. and Zask, A., Phosphatidylinositol 3-kinase (PI3K) inhibitors as anticancer drugs, Drugs Fut. 2007, 32(6): 537-547,
Although it seems clear that inhibition of the a isoform is essential for the antitumor activity of PI3K inhibitors, it is not clear whether a more selective inhibitor of a particular PI3K isoform may lead to fewer unwanted biological effects. It has recently been reported that non-PI3Kα class I isoforms (PI3Kβ, δ and γ) have the ability to induce oncogenic transformation of cells, suggesting that nonisoform-specific inhibitors may offer enhanced therapeutic potential over specific inhibitors.
Selectivity versus other related kinases is also an important consideration for the development of PI3K inhibitors. While selective inhibitors may be preferred in order to avoid unwanted side effects, there have been reports that inhibition of multiple targets in the PI3K/Akt pathway (e.g., PI3Kα and mTOR [mammalian target of rapamycin]) may lead to greater efficacy. It is possible that lipid kinase inhibitors may parallel protein kinase inhibitors in that nonselective inhibitors may also be brought forward to the clinic.

Mammalian Target of Rapamycin, mTOR, is a cell-signaling protein that regulates the response of tumor cells to nutrients and growth factors, as well as controlling tumor blood supply through effects on Vascular Endothelial Growth Factor, VEGF. Inhibitors of mTOR starve cancer cells and shrink tumors by inhibiting the effect of mTOR. All mTOR inhibitors bind to the mTOR kinase. This has at least two important effects. First, mTOR is a downstream mediator of the PI3K/Akt pathway. The PI3K/Akt pathway is thought to be over-activated in numerous cancers and may account for the widespread response from various cancers to mTOR inhibitors. The over-activation of the upstream pathway would normally cause mTOR kinase to be over-activated as well. However, in the presence of mTOR inhibitors, this process is blocked. The blocking effect prevents mTOR from signaling to downstream pathways that control cell growth. Over-activation of the PI3K/Akt kinase pathway is frequently associated with mutations in the PTEN gene, which is common in many cancers and may help predict what tumors will respond to mTOR inhibitors. The second major effect of mTOR inhibition is anti-angiogenesis, via the lowering of VEGF levels.

In lab tests, certain chemotherapy agents were found to be more effective in the presence of mTOR inhibitors. George, J. N., et al., Cancer Research, 61, 1527-1532, 2001. Additional lab results have shown that some rhabdomyosarcoma cells die in the presence of mTOR inhibitors. The complete functions of the mTOR kinase and the effects of mTOR inhibition are not completely understood.

There are three mTOR inhibitors, which have progressed into clinical trials. These compounds are Wyeth's Torisel, also known as 42-(3-hydroxy-2-(hydroxymethyl)-rapamycin 2-methylpropanoate, CCl-779 or Temsirolimus; Novartis' Everolimus, also known as 42-O-(2-hydroxyethyl)-rapamycin, or RAD 001; and Ariad's AP23573 also known as 42-(dimethylphopsinoyl)-rapamycin. The FDA has approved Torisel for the treatment of advanced renal cell carcinoma. In addition, Torisel is active in a NOS/SCID xenograft mouse model of acute lymphoblastic leukemia [Teachey et al, Blood, 107(3), 1149-1155, 2006]. On Mar. 30, 2009, the Food and Drug Administration (FDA) approved Everolimus (AFINITOR™) for the treatment of patients with advanced renal cell carcinoma. AP23573 has been given orphan drug and fast-track status by the FDA for treatment of soft-tissue and bone sarcomas.

The three mTOR inhibitors have non-linear, although reproducible pharmacokinetic profiles. Mean area under the curve (AUC) values for these drugs increase at a less than dose related way. The three compounds are all semi-synthetic derivatives of the natural macrolide antibiotic rapamycin. It would be desirable to find fully synthetic compounds, which inhibit mTOR that are more potent and exhibit improved pharmacokinetic behaviors.

There are three dual PI3K/mTOR inhibitors, which have progressed into phase I clinical trials. These compounds are Novartis's BTG-226, Exelixis's XL-765, and Novartis's BEZ-235, also known as 2-methyl-2-[4-(3-methyl-2-oxo-8-quinolin-3-yl-2,3-dihydro-imidazo[4,5-c]quinolin-1-yl)-phenyl]-propionitrile.

There are seven PI3K inhibitors, which have progressed into phase 1 clinical trials. These compounds are BKM-120 from Novartis, SF 1126, also known as N²-(1,4-dioxo-4-((4-(4-oxo-8-phenyl-4H-1-benzopyran-2-yl)morpholinium-4-yl)methoxy)butyl)-L-arginylglycyl-L-α-aspartyl-L-serine inner salt from Semafore, XL-147 from Exelixis, CAL-101 from Calistoga, GDC-0941, also known as 2-(1H-indazol-4-yl)-6-(4-methanesulfonylpiperazin-1-ylmethyl)-4-morpholin-4-ylthieno(3,2-d)pyrimidine from Genetech/Roche/Piramed, GSK-1059615 from GlaxoSmithKline, and PX-866, (1S,4E,10R,11R,13S,14R)-[4-diallylaminomethylene-6-hydroxy-1-methoxymethyl-10,13-dimethyl-3,7,17-trioxo-1,3,4,7,10,11,12,13,14,15,16,17-dodecahydro-2-oxa-cyclopenta[a]phenanthren-11-yl acetic acid ester a semi-synthetic viridin analogue, from Oncothyreon. The GSK-1059615 study was terminated prematurely due to lack of sufficient exposure following single- and repeat-dosing.

As explained above, PI3K inhibitors and mTOR inhibitors are expected to be novel types of medicaments useful against cell proliferation disorders, especially as carcinostatic agents. Thus, it would be advantageous to have new PI3K inhibitors and mTOR inhibitors as potential treatment regimens for mTOR- and PI3K-related diseases.

### SUMMARY OF THE INVENTION

In one aspect, the invention provides compounds of the formula I:

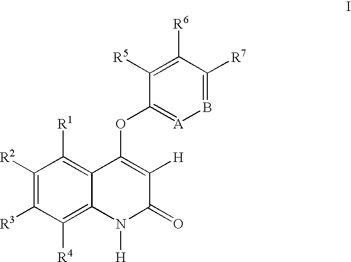

and pharmaceutically acceptable salts thereof, wherein A, B, R1, R2, R3, R4, R5, R6, and R7 are defined as set forth below.

The invention also provides pharmaceutical compositions comprising the compounds of the invention and a pharmaceutically acceptable carrier.

The invention further provides methods for making the compounds of the invention, and methods of using such compounds, as described below. In one aspect, the compounds and compositions of this invention are useful for inhibiting mTOR kinase. In another aspect, the compounds and compositions of this invention are useful for inhibiting PI3 kinases (PI3K's). In a further aspect, the compounds and compositions of this invention are useful for treating cancers.

### DETAILED DESCRIPTION OF THE INVENTION

In one aspect, the invention provides compounds of the Formula I:

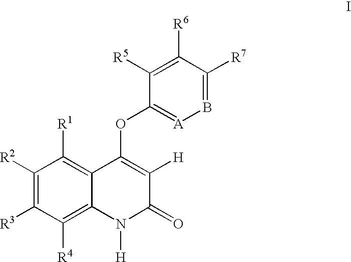

wherein:

A and B are each independently CH or N;

R¹, R², R³, and R⁴ are each independently selected from:

H, halogen, C₆-C₁₀aryl, C₁-C₉heteroaryl, and C₁-C₉heterocyclyl-, C₁-C₆alkyl, C₃-C₆cycloalkyl, C₂-C₆alkenyl, C₂-C₆alkynyl, NHC(O)Y, NR′R″, CF₃, OCF₃, OCHF₂, OR′, SR′, SOR′, SO₂R′, NO₂, SO₂NR′R″, NHSO₂R′, CONR′R″, CN, COOR′, OCOR′, COR′, NHCONR′R″, and NHCOOR';

Y is C₁-C₉heteroaryl-, C₁-C₆alkyl-, or C₁-C₉heterocyclyl- and is optionally substituted with 1 or 2 substituents selected from C₆-C₁₀aryl-, C₁-C₉heteroaryl-, C₁-C₉heterocyclyl-, OR′, SR′, COR′, and NR′R″;

R′ and R″ are each independently selected from the group consisting of H, C₁-C₆alkyl, C₆-C₁₀aryl-, C₁-C₉heteroaryl-, and C₁-C₉heterocyclyl-;

R⁵ and R⁷ are each independently selected from the group consisting of H, HO—, C₁-C₆alkoxy, C₆-C₁₀aryl-O—, C₁-C₉heteroaryl-O—, C₁-C₉heterocyclyl-O—, NHC(O)Y, C₆-C₁₀aryl, C₁-C₉heteroaryl, and C₁-C₉heterocyclyl-, COR′, OCF₃, C₁-C₆alkyl, C₃-C₆cycloalkyl, C₂-C₆alkenyl, C₂-C₆alkynyl, OCHF₂, halogen, COOR′, NO₂, NR′R″, OH, CF₃, and CN;

R⁶ is selected from the group consisting of H, HO—, C₁-C₆alkoxy, C₆-C₁₀aryl-O—, C₁-C₉heteroaryl-O—, C₁-C₉heterocyclyl-O—, NHC(O)Y, C₆-C₁₀aryl, C₁-C₉heteroaryl, C₁-C₉heterocyclyl-, COR′, OCF₃, C₁-C₆alkyl, C₃-C₆cycloalkyl, C₂-C₆alkenyl, C₂-C₆alkynyl, OCHF₂, halogen, COOR′, NO₂, NR′R″, NHCHR′R″, CF₃, CN, and CH₂CN;

or R⁵ and R⁶ taken together with the carbon atoms to which they are attached form a C₆-C₁₀aryl;

or, R⁶ and R⁷ taken together are OCH₂O, so that together with the carbon atoms to which they are attached they form a 5-membered ring;

each C₁-C₆alkyl group may be optionally substituted by a OH, NR′R″, C₃-C₆cycloalkyl, C₆-C₁₀aryl, C₁-C₉heteroaryl, or C₁-C₉heterocyclyl-group;

each C₁-C₉heteroaryl- and C₁-C₉heterocyclyl-group includes 1-4 ring atoms selected from O, S, and N;

and each C₆-C₁₀aryl, C₁-C₉heteroaryl, and C₁-C₉heterocyclyl-group is optionally substituted with one to three substituents independently selected from halogen, C₆-C₁₀aryl, C₁-C₉heteroaryl, C₁-C₉heterocyclyl-, C₁-C₆alkyl, C₃-C₆cycloalkyl, C₂-C₆alkenyl, C₂-C₆alkynyl, NHC(O)Y, NR′R″, CF₃, OCF₃, OCHF₂, OR′, SR′, SOR′, SO₂R′, NO₂, SO₂NR′R″, NHSO₂R′, CONR′R″, CN, COOR′, OCOR′, COR′, NHCONR′R″, and NHCOOR';

provided that: when A and B are both CH, and R⁵, R⁶ and R⁷ are all H, then R¹, R², R³ and R⁴ are not H, halogen, phenyl, naphthyl, C₁-C₆alkyl, C₃-C₆cycloalkyl, C₂-C₆alkenyl, NHC(O)Y, NR′R″, OR′, SR′, NO₂, SO₂NR′R″, NHSO₂R′, CONR′R″, CN, COOR′, OCOR′, COR′, or NHCONR′R″;

further provided that: when A and B are both CH, and R¹, R², R³, R⁴, R⁵, and R⁶ are all H, then R⁷ cannot be H, unsubstituted methyl or unsubstituted methoxy;

and further provided that: when B is N, then R¹, R², R³ and R⁴ are not NO₂, CN, COOR′, CONR′R″, C₃-C₆cycloalkyl-, or C₁-C₉heterocyclyl-,

and pharmaceutically acceptable salts thereof.

In certain embodiments of the invention, A and B are both CH; in other embodiments, one of A and B is N and the other is CH.

In some embodiments of the invention, one or more of R¹, R², R³, and R⁴ may be H.

In some embodiments, R² or R³ may be selected from the group consisting of halogen, C₆-C₁₀aryl-, C₁-C₉heteroaryl-, C₁-C₉heterocyclyl-, and NHC(O)Y.

In certain embodiments, R⁵ is halogen, C₁-C₆alkoxy-, or CF₃, or R⁵ and R⁶ taken together with the carbon atoms to which they are attached form a phenyl ring. In some embodiments, R⁶ is selected from the group consisting of C₆-C₁₀aryl, C₁-C₉heteroaryl, C₁-C₉heterocyclyl-, and NCHR′R″.

In some embodiments, R¹, R², R³, R⁴, R⁵, and R⁷ are all H. In some of these embodiments, A and B are both CH and R⁶ is aryl or C₁-C₉heteroaryl, for example substituted or unsubstituted phenyl, pyrazolyl or pyridyl, or R⁶ is NR′R″, for example NHCH₂heteroaryl. In other of these embodiments, one of A and B is N and the other is CH and R⁶ is H.

In one embodiment, R¹ is H.

In one embodiment, R⁴ is H.

In one embodiment, R³ is H.

In one embodiment, R² is H.

In one embodiment, R² or R³ is selected from the group consisting of halogen, C₆-C₁₀aryl,

C₁-C₉heteroaryl, C₁-C₉heterocyclyl-, and NHC(O)Y.

In one embodiment, A and B are both CH.

In one embodiment, A is CH and B is N.

In one embodiment, A is N and B is CH.

In one embodiment, R⁵ is halogen, C₁-C₆alkoxy-, or CF₃, or R⁵ and R⁶ taken together with the carbon atoms to which they are attached form a phenyl ring.

In one embodiment, R⁶ is selected from the group consisting of C₆-C₁₀aryl, C₁-C₉heteroaryl, C₁-C₉heterocyclyl-, and NHCHR′R″.

In one embodiment, R¹, R², R³, R⁴, R⁵, and R⁷ are all H.

In one embodiment, A and B are both CH and R⁶ is selected from the group consisting of C₆-C₁₀aryl, C₁-C₉heteroaryl, C₁-C₉heterocyclyl-, and NHCHR′R″.

In one embodiment, one of A and B is N and the other is CH.

In one embodiment, R⁶ is H.

Illustrative compounds of formula I are:
4-[3-(6-aminopyridin-3-yl)phenoxy]quinolin-2(1H)-one;
4-({4′-[(1-methylpiperidin-4-yl)amino]biphenyl-3-yl}oxy)quinolin-2(1H)-one;
4-{3-[1-(2-morpholin-4-ylethyl)-1H-pyrazol-4-yl]phenoxy}quinolin-2(1H)-one;
4-{3-[pyridin-3-ylmethyl)amino]phenoxy}quinolin-2(1H)-one;
4-(pyridin-2-yloxy) quinolin-2(1H)-one;
4-(pyridin-3-yloxy) quinolin-2(1H)-one;
2,2-dimethyl-N-{3-[({3-[(2-oxo-1,2-dihydroquinolin-4-yl)oxy]phenyl}amino)methyl]-pyridin-2-yl}propanamide;
4-(3-{[(5-fluoropyridin-3-yl)methyl]amino}phenoxy)quinolin-2(1H)-one;
4-({4′-[(1-methylpiperidin-4-yl)amino]biphenyl-3-yl}oxy)quinolin-2(1H)-one;
4-(3-{[(6-chloropyridin-3-yl)methyl]amino}phenoxy)quinolin-2(1H)-one;
4-{3-[(quinolin-3-ylmethyl)amino]phenoxy}quinolin-2(1H)-one;
4-(3-{[(2-aminopyridin-3-yl)methyl]amino}phenoxy)quinolin-2(1H)-one;
4-(3-{6-[3-(dimethylamino)propoxy]pyridin-3-yl}phenoxy)quinolin-2(1H)-one;
4-(3-{[4-(1H-imidazol-1-yl)benzyl]amino}phenoxy)quinolin-2(1H)-one;
4-{3-[(pyridin-3-ylmethyl)amino]phenoxy}quinolin-2(1H)-one;
4-(3-{[(6-fluoropyridin-3-yl)methyl]amino}phenoxy)quinolin-2(1H)-one;
4-{3-[(2-thienylmethyl)amino]phenoxy}quinolin-2(1H)-one;
4-[3-({4-[6-(hydroxymethyl)pyridin-2-yl]benzyl}amino)phenoxy]quinolin-2(1H)-one;
4-{[4′-(piperidin-4-ylamino)biphenyl-3-yl]oxy}quinolin-2(1H)-one;
4-[3-({[2-(methylamino)pyridin-3-yl]methyl}amino)phenoxy]quinolin-2(1H)-one;
4-[3-(6-aminopyridin-3-yl)phenoxy]quinolin-2(1H)-one;
4-{3-[(4-pyridin-3-ylbenzyl)amino]phenoxy}quinolin-2(1H)-one; and
1-[2-(dimethylamino)ethyl]-3-{3′-[(2-oxo-1,2-dihydroquinolin-4-yl)oxy]biphenyl-4-yl}urea;
   or a pharmaceutically acceptable salt thereof.

In other aspects, the invention provides pharmaceutical compositions comprising compounds or pharmaceutically acceptable salts of the compounds of the present Formula I and a pharmaceutically acceptable carrier.

In other aspects, the invention provides that the pharmaceutically acceptable carrier suitable for oral administration and the composition comprises an oral dosage form.

In other aspects, the invention provides a composition comprising a compound of Formula I; a second compound selected from the group consisting of a topoisomerase I inhibitor, a MEK1/2 inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab, and lavendustin A; and a pharmaceutically acceptable carrier.

In one embodiment, the second compound is Avastin.

In other aspects, the invention provides a method of treating a PI3K-related disorder, comprising administering to a mammal in need thereof a compound of Formula I in an amount effective to treat a PI3K-related disorder.

In other aspects, the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the PI3K-related disorder is cancer.

In one embodiment, the cancer is selected from the group consisting of leukemia, skin cancer, bladder cancer, breast cancer, uterus cancer, ovarian cancer, prostate cancer, non-small cell lung cancer, colon cancer, pancreas cancer, renal cancer, gastric cancer, and brain cancer.

In other aspects, the invention provides a method of treating an mTOR-related disorder, comprising administering to a mammal in need thereof a compound of Formula I in an amount effective to treat an mTOR-related disorder.

In other aspects, the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the mTOR-related disorder is cancer.

In one embodiment, the cancer is selected from the group consisting of leukemia, skin cancer, bladder cancer, breast cancer, uterus cancer, ovarian cancer, prostate cancer, non-small cell lung cancer, colon cancer, pancreas cancer, renal cancer, gastric cancer, and brain cancer.

In one embodiment, the invention provides a method of treating advanced renal cell carcinoma, comprising administering to a mammal in need thereof a compound of Formula I in an amount effective to treat advanced renal cell carcinoma.

In other aspects, the invention provides a method of treating acute lymphoblastic leukemia, comprising administering to a mammal in need thereof a compound of Formula I in an amount effective to treat acute lymphoblastic leukemia.

In one embodiment, the invention provides a method of treating acute malignant melanoma, comprising administering to a mammal in need thereof a compound of Formula I in an amount effective to treat malignant melanoma.

In one embodiment, the invention provides a method of treating soft-tissue or bone sarcoma, comprising administering to a mammal in need thereof a compound of Formula I in an amount effective to treat soft-tissue or bone sarcoma.

In one embodiment, the invention provides a method of treating a cancer selected from the group consisting of leukemia, skin cancer, bladder cancer, breast cancer, uterus cancer, ovarian cancer, prostate cancer, non-small cell lung cancer, colon cancer, pancreas cancer, renal cancer, gastric cancer, and brain cancer comprising administering to a mammal in need thereof a composition comprising a compound of Formula I; a second compound selected from the group consisting of a topoisomerase I inhibitor, a MEK1/2 inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab, and lavendustin A; and a pharmaceutically acceptable carrier. in an amount effective to treat the cancer.

In other aspects, the invention provides a method of inhibiting mTOR in a subject, comprising administering to a subject in need thereof a compound of Formula I in an amount effective to inhibit mTOR.

In other aspects, the invention provides a method of inhibiting PI3K in a subject, comprising administering to a subject in need thereof a compound of Formula I in an amount effective to inhibit PI3K.

In other aspects, the invention provides a method of inhibiting mTOR and PI3K together in a subject, comprising administering to a subject in need thereof a compound of Formula I in an amount effective to inhibit mTOR and PI3K.

In other aspects, the invention provides a method of synthesizing a compound of Formula I, comprising treating a compound of the formula II

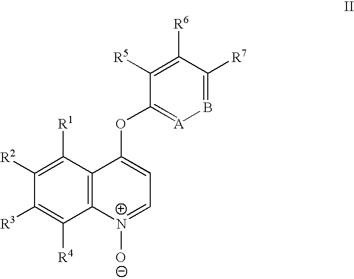

with a compound selected from tosyl chloride or benzoyl chloride, followed by hydrolysis with a base, wherein A, B, R¹, R², R³, R⁴, R⁵, R⁶ and R⁷ are defined as in Formula I.

In one embodiment, the base is selected from the group consisting of sodium carbonate, potassium carbonate, potassium hydroxide, sodium hydroxide, and triethylamine.

In other aspects, the compound of formula II is made by reacting a compound of the formula:

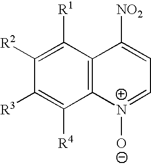

with a compound of the formula:

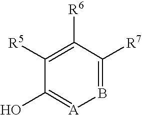

under basic conditions.

In other aspects, the invention provides a method of synthesizing a compound of Formula I, wherein A and B are both CH, comprising replacing the iodine atom in a compound of formula III:

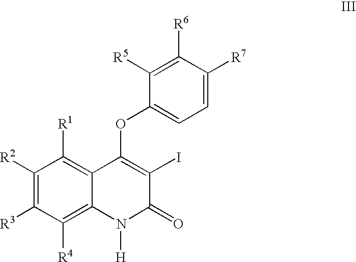

with a hydrogen atom.

In one embodiment, the iodine atom is replaced with a hydrogen atom by treating the compound of formula III with Zn and acetic acid.

In other aspects, the compound of formula III is prepared by a process comprising reacting a compound of formula:

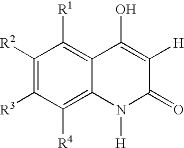

with a compound of formula:

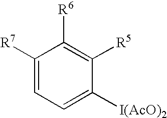

under basic conditions.

In one embodiment, R¹ and R⁴ are both H.

Representative “pharmaceutically acceptable salts” include but are not limited to, e.g., water-soluble and water-insoluble salts, such as the acetate, aluminum, amsonate (4,4-diaminostilbene-2,2-disulfonate), benzathine (N,N′-dibenzylethylenediamine), benzenesulfonate, benzoate, bicarbonate, bismuth, bisulfate, bitartrate, borate, bromide, butyrate, calcium, calcium edetate, camsylate (camphorsulfonate), carbonate, chloride, choline, citrate, clavulariate, diethanolamine, dihydrochloride, diphosphate, edetate, edisylate (camphorsulfonate), esylate (ethanesulfonate), ethylenediamine, fumarate, gluceptate (glucoheptonate), gluconate, glucuronate, glutamate, hexafluorophosphate, hexylresorcinate, hydrabamine (N,N′-bis(dehydroabietyl)ethylenediamine), hydrobromide, hydrochloride, hydroxynaphthoate, 1-hydroxy-2-naphthoate, 3-hydroxy-2-naphthoate, iodide, isothionate (2-hydroxyethanesulfonate), lactate, lactobionate, laurate, lauryl sulfate, lithium, magnesium, malate, maleate, mandelate, meglumine (1-deoxy-1-(methylamino)-D-glucitol), mesylate, methyl bromide, methylnitrate, methylsulfate, mucate, napsylate, nitrate, N-methylglucamine ammonium salt, oleate, oxalate, palmitate, pamoate (4,4′-methylenebis-3-hydroxy-2-naphthoate, or embonate), pantothenate, phosphate, picrate, polygalacturonate, potassium, propionate, p-toluenesulfonate, salicylate, sodium, stearate, subacetate, succinate, sulfate, sulfosaliculate, suramate, tannate, tartrate, teoclate (8-chloro-3,7-dihydro-1,3-dimethyl-1H-purine-2,6-dione), triethiodide, tromethamine (2-amino-2-(hydroxymethyl)-1,3-propanediol), valerate, and zinc salts.

Some compounds within the present invention possess one or more chiral centers, and the present invention includes each separate enantiomer of such compounds as well as mixtures of the enantiomers. Where multiple chiral centers exist in compounds of the present invention, the invention includes each combination as well as mixtures thereof. All chiral, diastereomeric, and racemic forms of a structure are intended, unless the specific stereochemistry or isomeric form is specifically indicated. It is well known in the art how to prepare optically active forms, such as by resolution of racemic forms or by synthesis from optically active starting materials.

An “effective amount” when used in connection a compound of the present invention of this invention is an amount effective for inhibiting mTOR or PI3K in a subject.

### DEFINITIONS

The following definitions are used in connection with the compounds of the present invention unless the context indicates otherwise. In general, the number of carbon atoms present in a given group is designated “Cₓ-C_{y}”, where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions and the like. Unless indicated otherwise, the nomenclature of substituents that are not explicitly defined herein are arrived at by naming from left to right the terminal portion of the functionality followed by the adjacent functionality toward the point of attachment. For example, the substituent “arylalkyloxycabonyl” refers to the group (C₆-C₁₄aryl)-(C₁-C₆alkyl)-O—C(O)—. It is understood that the above definitions are not intended to include impermissible substitution patterns (e.g., methyl substituted with 5 fluoro groups, two hydroxyl groups on a single carbon atom, a hydroxyl group on a non-aromatic double bond). Such impermissible substitution patterns are well known to the skilled artisan. In each of the below groups, when a subgroup is designated with a multiple occurrence, each occurrence is selected independently. For example, in di(C₁-C₆alkyl)amino- e.g. (C₁-C₆alkyl)₂N—, the C₁-C₆alkyl groups can be the same or different. Preferably C₁-C₆alkyl, C₆-C₁₄aryl, C₁-C₉heteroaryl, and C₁-C₉heterocyclyl substituent groups, if substituted themselves, are substituted by a substituent that is unsubstituted or at most mono- or di-substituted.

“Alkoxy-” refers to the group R—O— where R is an alkyl group, as defined below. Exemplary C₁-C₆alkoxy-groups include but are not limited to methoxy, ethoxy, n-propoxy, 1-propoxy, n-butoxy and t-butoxy. An alkoxy group can be unsubstituted or substituted with one or more of the following groups: halogen, hydroxyl, C₁-C₆alkoxy-, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, C₁-C₆alkoxy-, HO₂O—, (C₁-C₆alkoxy)carbonyl-, C₁-C₈acyl-, C₆-C₁₄aryl-, C₁-C₉heteroaryl-, C₃-C₈cycloalkyl-, C₁-C₆haloalkyl-, C₁-C₆-aminoalkyl-, (C₁-C₆alkyl)carboxy-, C₁-C₆-carbonylamidoalkyl-, or O₂N—.

“Alkyl-” refers to a hydrocarbon chain that may be a straight chain or branched chain, containing the indicated number of carbon atoms, for example, a O₁—O₁₀alkyl-group may have from 1 to 10 (inclusive) carbon atoms in it. In the absence of any numerical designation, “alkyl” is a chain (straight or branched) having 1 to 6 (inclusive) carbon atoms in it. Examples of C₁-C₆alkyl-groups include, but are not limited to, methyl, ethyl, propyl, butyl, pentyl, hexyl, isopropyl, isobutyl, sec-butyl, tert-butyl, isopentyl, neopentyl, and isohexyl. An alkyl-group can be unsubstituted or substituted with one or more of the following groups: halogen, H₂N—, (C₁-C₆alkyl)amino-, di(C₁-C₆alkyl)amino-, (C₁-C₆alkyl)O(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂O—, (C₁-C₆alkoxy)carbonyl-, C₁-C₈acyl-, C₆-C₁₄aryl-, C₁-C₉heteroaryl-, C₃-C₈cycloalkyl-, C₁-C₆haloalkyl-, C₁-C₆-aminoalkyl-, (C₁-C₆alkyl)carboxy-, C₁-C₆-carbonylamidoalkyl-, or O₂N—.

“Alkenyl-” refer to a straight or branched chain unsaturated hydrocarbon containing at least one double bond. Where E- and/or Z-isomers are possible, the term “alkenyl” is intended to include all such isomers. Examples of a C₂-C₆alkenyl-group include, but are not limited to, ethylene, propylene, 1-butylene, 2-butylene, isobutylene, sec-butylene, 1-pentene, 2-pentene, isopentene, penta-1,4-dien-1-yl, 1-hexene, 2-hexene, 3-hexene, and isohexene. An alkenyl-group can be unsubstituted or substituted with one or more of the following groups: halogen, H₂N—, (C₁-C₆alkyl)amino-, di(C₁-C₆alkyl)amino-, (C₁-C₆alkyl)O(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂O—, (C₁-C₆alkoxy)carbonyl-, C₁-C₈acyl-, C₆-C₁₄aryl-, C₁-C₉heteroaryl-, and C₃-C₈cycloalkyl-.

“Alkynyl-” refers to a straight or branched chain unsaturated hydrocarbon containing at least one triple bond. Examples of a C₂-C₆alkynyl-group include, but are not limited to, acetylene, propyne, 1-butynyl, 2-butynyl, isobutynyl, sec-butynyl, 1-pentynyl, 2-pentynyl, isopentynyl, penta-1,4-diyn-1-yl, 1-hexynyl, 2-hexynyl, 3-hexynyl, and isohexynyl. An alkynyl group can be unsubstituted or substituted with one or more of the following groups: halogen, H₂N—, (C₁-C₆alkyl)amino-, di(C₁-C₆alkyl)amino-, (C₁-C₆alkyl)O(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂O—, (C₁-C₆alkoxy)carbonyl-, C₁-C₈acyl-, C₆-C₁₄aryl-, C₁-C₉heteroaryl-, and C₃-C₈cycloalkyl-.

Aryl- refers to an aromatic hydrocarbon group. Examples of an C₆-C₁₄aryl-group include, but are not limited to, phenyl, 1-naphthyl, 2-naphthyl, 3-biphen-1-yl, anthryl, tetrahydronaphthyl, fluorenyl, indanyl, biphenylenyl, and acenaphthenyl. Examples of an C₆-C₁₀aryl-group include, but are not limited to, phenyl, 1-naphthyl, 2-naphthyl, tetrahydronaphthyl, and indanyl. An aryl group can be monocyclic or polycyclic as long as at least one ring is aromatic and the point of attachment is at an aromatic carbon atom. An aryl group can be unsubstituted or substituted with one or more of the following groups: C₁-C₆alkyl-, halogen, haloalkyl-, hydroxyl, hydroxyl(C₁-C₆alkyl)-, H₂N—, aminoalkyl-, di(C₁-C₆alkyl)amino-, HO₂C—, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“Cycloalkyl-” refers to a monocyclic saturated hydrocarbon ring. Representative examples of a C₃-C₈cycloalkyl- include, but are not limited to, cyclopropyl, cyclobutyl, cyclopentyl, cyclohexyl, cycloheptyl, and cyclooctyl. Representative examples of a C₃-C₆cycloalkyl- include, but are not limited to, cyclopropyl, cyclobutyl, cyclopentyl, and cyclohexyl. A cycloalkyl- can be unsubstituted or independently substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, or C₃-C₈cycloalkyl-, C₁-C₆haloalkyl-, C₁-C₆-aminoalkyl-, (C₁-C₆alkyl)carboxy-, C₁-C₆-carbonylamidoalkyl-, or O₂N—. Additionally, each of any two hydrogen atoms on the same carbon atom of the carbocyclic ring can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle-containing two oxygen atoms.

“Halo” or “halogen” refers to —F, —Cl, —Br and —I.

“Heteroaryl-” refers to 5-10-membered mono and bicyclic aromatic groups containing at least one heteroatom selected from oxygen, sulfur, and nitrogen, wherein any S can optionally be oxidized, and any N can optionally be quaternized with an C₁-C₆alkyl group. Examples of monocyclic C₁-C₉heteroaryl-radicals include, but are not limited to, oxazinyl, thiazinyl, diazinyl, triazinyl, thiadiazolyl, tetrazinyl, imidazolyl, tetrazolyl, isoxazolyl, furanyl, furazanyl, oxazolyl, thiazolyl, thiophenyl, pyrazolyl, triazolyl, pyrimidinyl, N-pyridyl, 2-pyridyl, 3-pyridyl and 4-pyridyl. Examples of bicyclic C₁-C₉heteroaryl-radicals include but are not limited to, benzimidazolyl, indolyl, isoquinolinyl, benzofuranyl, benzothiophenyl, indazolyl, quinolinyl, quinazolinyl, purinyl, benzisoxazolyl, benzoxazolyl, benzthiazolyl, benzodiazolyl, benzotriazolyl, isoindolyl, and indazolyl. The contemplated heteroaryl-rings or ring systems have a minimum of 5 members. Therefore, for example, C₁heteroaryl-radicals would include but are not limited to tetrazolyl, C₂heteroaryl-radicals include but are not limited to triazolyl, thiadiazolyl, and tetrazinyl, C₉heteroaryl-radicals include but are not limited to quinolinyl and isoquinolinyl. A heteroaryl-group can be unsubstituted or substituted with one or more of the following groups: C₁-C₆alkyl-, halogen, C₁-C₆haloalkyl-, hydroxyl, C₁-C₆hydroxylalkyl-, H₂N—, C₁-C₆-aminoalkyl-, di(C₁-C₆alkyl)amino-, —COOH, (C₁-C₆alkoxy)carbonyl-, (C₁-C₆alkyl)carboxy-, di(C₁-C₆alkylamido-, H₂NC(O)—, (C₁-C₆alkyl)amido-, or O₂N—.

The term “heteroatom” refers to a sulfur, nitrogen, or oxygen atom.

“Heterocycle” or “heterocyclyl-” refers to 3-10-membered monocyclic, fused bicyclic, and bridged bicyclic groups containing at least one heteroatom selected from oxygen, sulfur and nitrogen, wherein any S can optionally be oxidized, and any N can optionally be quaternized by a C₁-C₆alkyl group. A heterocycle may be saturated or partially saturated. One of the rings for a fused bicyclic heterocycle may be aromatic. Exemplary C₁-C₉heterocyclyl-groups include but are not limited to aziridine, oxirane, oxirene, thiirane, pyrroline, pyrrolidine, dihydrofuran, tetrahydrofuran, dihydrothiophene, tetrahydrothiophene, dithiolane, piperidine, 1,2,3,6-tetrahydropyridine-1-yl, tetrahydropyran, pyran, thiane, thiine, piperazine, oxazine, 5,6-dihydro-4H-1,3-oxazin-2-yl, 2,5-diazabicyclo[2.2.1]heptane, 2,5-diazabicyclo[2.2.2]octane, 3,6-diazabicyclo[3.1.1]heptane, 3,8-diazabicyclo[3.2.1]octane, 6-oxa-3,8-diazabicyclo[3.2.1]octane, 7-oxa-2,5-diazabicyclo[2.2.2]octane, 2,7-dioxa-5-azabicyclo[2.2.2]octane, 2-oxa-5-azabicyclo[2.2.1]heptane, 2-oxa-5-azabicyclo[2.2.2]octane, 3,6-dioxa-8-azabicyclo[3.2.1]octane, 3-oxa-6-azabicyclo[3.1.1]heptane, 3-oxa-8-azabicyclo[3.2.1]octane, 5,7-dioxa-2-azabicyclo[2.2.2]octane, 6,8-dioxa-3-azabicyclo[3.2.1]octane, 6-oxa-3-azabicyclo[3.1.1]heptane, 8-oxa-3-azabicyclo[3.2.1]octane, 8-oxa-3-azabicyclo[3.2.1]octan-3-yl, 2-methyl-2,5-diazabicyclo[2.2.1]heptane-5-yl, 1,3,3-trimethyl-6-azabicyclo[3.2.1]oct-6-yl, 4-methyl-3,4-dihydro-2H-1,4-benzoxazin-7-yl, thiazine, dithiane, and dioxane. The contemplated heterocycle rings or ring systems have a minimum of 3 members. Therefore, for example, C₁heterocyclyl-radicals would include but are not limited to oxaziranyl, diaziridinyl, and diazirinyl, C₂heterocyclyl-radicals include but are not limited to aziridinyl, oxiranyl, and diazetidinyl, C₉heterocyclyl-radicals include but are not limited to azecanyl, tetrahydroquinolinyl, and perhydroisoquinolinyl.

A “subject” is a mammal, e.g., a human, mouse, rat, guinea pig, dog, cat, horse, cow, pig, or non-human primate, such as a monkey, chimpanzee, baboon or gorilla.

The compounds of the present invention exhibit an mTOR inhibitory activity and, therefore, can be utilized to inhibit abnormal cell growth in which mTOR plays a role. Thus, the compounds of the present invention are effective in the treatment of disorders with which abnormal cell growth actions of mTOR are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovarian cancer, prostate cancer, non-small cell lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

The compounds of the present invention exhibit a PI3 kinase inhibitory activity and therefore, can be utilized in order to inhibit abnormal cell growth in which PI3 kinases play a role. Thus, the compounds of the present invention are effective in the treatment of disorders with which abnormal cell growth actions of PI3 kinases are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovarian cancer, prostate cancer, non-small cell lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

The compounds of the present invention may inhibit both mTOR and PI3 kinase simultaneously and, therefore, can be utilized in order to inhibit abnormal cell growth in which both mTOR and PI3 kinases simultaneously play a role. Thus, the compounds of the present invention are effective in the treatment of disorders with which abnormal cell growth actions of PI3 kinases are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovarian cancer, prostate cancer, non-small cell lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

For therapeutic use, the pharmacologically active compounds of Formula I will normally be administered as a pharmaceutical composition comprising as the (or an) essential active ingredient at least one such compound in association with a solid or liquid pharmaceutically acceptable carrier and, optionally, with pharmaceutically acceptable adjutants and excipients employing standard and conventional techniques.

The pharmaceutical compositions of this invention include suitable dosage forms for oral, parenteral (including subcutaneous, intramuscular, intradermal and intravenous) bronchial or nasal administration. Thus, if a solid carrier is used, the preparation may be made into tablets, placed in a hard gelatin capsule in powder or pellet form, or in the form of a troche or lozenge. The solid carrier may contain conventional excipients such as binding agents, fillers, lubricants used to make tablets, disintegrants, wetting agents and the like. The tablet may, if desired, be film coated by conventional techniques. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. Particularly useful is the administration of a compound of Formula I directly in parenteral formulations. The pharmaceutical compositions are prepared by conventional techniques appropriate to the desired preparation containing appropriate amounts of the active ingredient, that is, the compound of Formula I according to the invention. See, for example, Remington: The Science and Practice of Pharmacy, 20th Edition. Baltimore, Md.: Lippincott Williams & Wilkins, 2000.

The dosage of the compound of Formula I to achieve a therapeutic effect will depend not only on such factors as the age, weight and sex of the patient and mode of administration, but also on the degree of potassium channel activating activity desired and the potency of the particular compound being utilized for the particular disorder of disease concerned. It is also contemplated that the treatment and dosage of the particular compound may be administered in unit dosage form and that one skilled in the art would adjust the unit dosage form accordingly to reflect the relative level of activity. The decision as to the particular dosage to be employed (and the number of times to be administered per day is within the discretion of the physician, and may be varied by titration of the dosage to the particular circumstances of this invention to produce the desired therapeutic effect.

A suitable dose of a compound of Formula I or pharmaceutical composition thereof for a mammal, including man, suffering from, or likely to suffer from any condition as described herein is an amount of active ingredient from about 0.01 mg/kg to 10 mg/kg body weight. For parenteral administration, the dose may be in the range of 0.1 mg/kg to 1 mg/kg body weight for intravenous administration. For oral administration, the dose may be in the range about 0.1 mg/kg to 5 mg/kg body weight. The active ingredient will preferably be administered in equal doses from one to four times a day. However, usually a small dosage is administered, and the dosage is gradually increased until the optimal dosage for the host under treatment is determined.

However, it will be understood that the amount of the compound actually administered will be determined by a physician, in the light of the relevant circumstances including the condition to be treated, the choice of compound of be administered, the chosen route of administration, the age, weight, and response of the individual patient, and the severity of the patient's symptoms.

The amount of the compound of the present invention or a pharmaceutically acceptable salts thereof is an amount that is effective for inhibiting mTOR or PI3K in a subject. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound of the present invention or a pharmaceutically acceptable salt thereof is administered, the effective dosage amounts correspond to the total amount administered.

In one embodiment, the compound of the present invention or a pharmaceutically acceptable salt thereof is administered concurrently with another therapeutic agent.

In one embodiment, a composition comprising an effective amount of a compound of the present invention or a pharmaceutically acceptable salt thereof and an effective amount of another therapeutic agent within the same composition can be administered.

Effective amounts of the other therapeutic agents are well known to those skilled in the art. However, it is well within the skilled artisan's purview to determine the other therapeutic agent's optimal effective amount range. The compound of the present invention or a pharmaceutically acceptable salt thereof and the other therapeutic agent can act additively or, in one embodiment, synergistically. In one embodiment, of the invention, where another therapeutic agent is administered to an animal, the effective amount of the compound of the present invention or a pharmaceutically acceptable salt thereof is less than its effective amount would be where the other therapeutic agent is not administered. In this case, without being bound by theory, it is believed that the compound of the present invention or a pharmaceutically acceptable salt thereof and the other therapeutic agent act synergistically.

Procedures used to synthesize the compounds of the present invention are described in Schemes 1-21 and are illustrated in the examples. Reasonable variations of the described procedures are intended to be within the scope of the present invention:

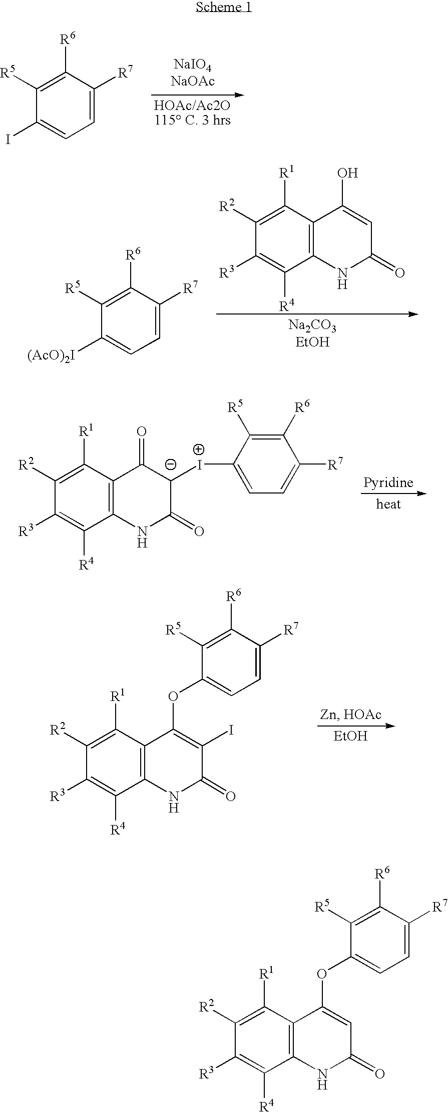

Compounds of this invention may be prepared according to Scheme 1. The iodides are oxidized with periodate to yield the iodonium salts, which are then reacted with the quinolinone compounds in aqueous sodium carbonate at 38° C. for 1 hour 20 minutes. The isolated salts are then heated with pyridine at 118° C. for 2 hours to yield the 3-iodo-4-phenoxy compounds, which are then reduced by heating with zinc in acetic acid for 3.5 hours.

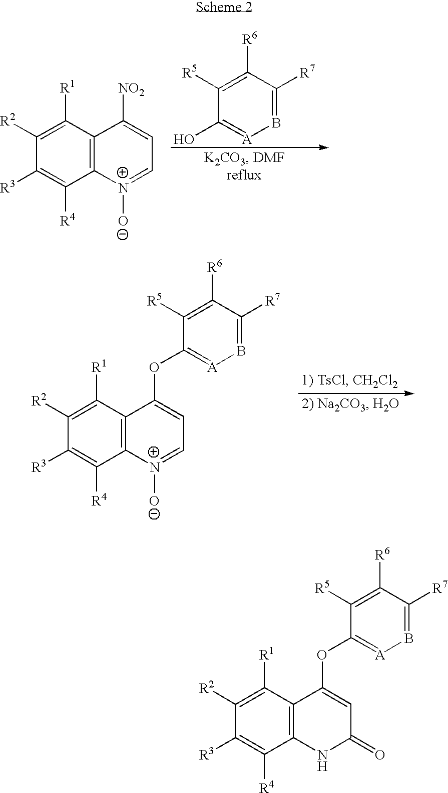

Compounds of this invention may be prepared according to Scheme 2. The nitro compounds are reacted with aryl alcohol and potassium carbonate in DMF under reflux. The resulting N-oxides are then reacted with tosyl chloride followed by hydrolysis with aqueous sodium carbonate.

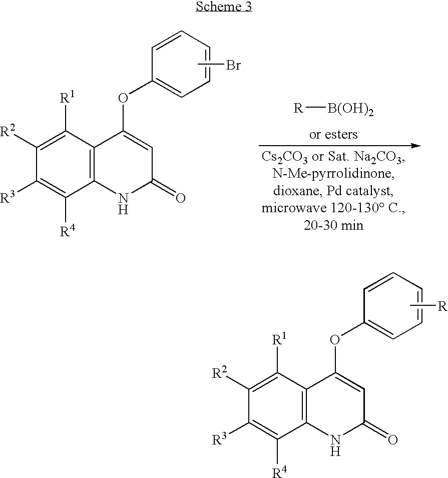

Compounds of this invention may be prepared according to Scheme 3 by reacting the bromides with boronic acid or boronate in the presence of base (such as cesium carbonate, or saturated aqueous sodium carbonate), a solvent (such as N-Me-pyrrolidinone or dioxane), Pd catalyst [such as 2-(dimethylamino)-2-biphenylpalladium (II) chloride dinorborylphosphine, or tetrakis(triphenylphosphine)palladium(0)] under microwave irradiation at 120 to 130° C. for 20-30 minutes. R represents any of R⁵, R⁶ and R⁷ is selected from the group C₆-C₁₀aryl, C₁-C₉heteroaryl, and C₂-C₆alkenyl.

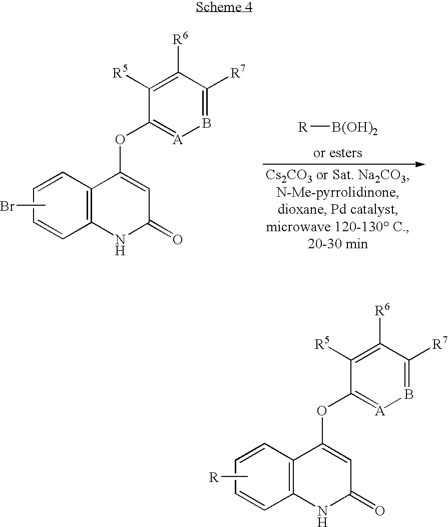

Compounds of this invention may be prepared according to Scheme 4 by reacting the bromides with boronic acid or boronate in the presence of base (such as cesium carbonate, or saturated aqueous sodium carbonate), a solvent (such as N-Me-pyrrolidinone or dioxane), Pd catalyst [such as 2-(dimethylamino)-2-biphenylpalladium (II) chloride dinorborylphosphine, or tetrakis(triphenylphosphine)palladium(0)] under microwave irradiation at 120 to 130° C. for 20-30 minutes. R represents any of R¹, R², R³, and R⁴, which are selected, from C₆-C₁₀aryl, C₁-C₉heteroaryl, or C₂-C₆alkenyl.

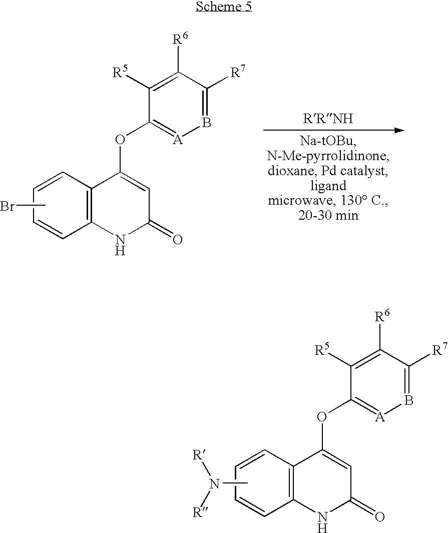

Compounds of this invention may be prepared according to Scheme 5 by reacting the bromides with the amines in the presence of base (such as potassium tert-butoxide), a solvent (such as N-Me-pyrrolidinone or dioxane), Pd catalyst (such as 2-(dimethylamino)-2-biphenylpalladium (II) chloride dinorborylphosphine), and a ligand [such as 1,3-bis(2,6-di-1-propylphenyl)imidazolium chloride] under microwave irradiation at 130° C. for 20-30 minutes.

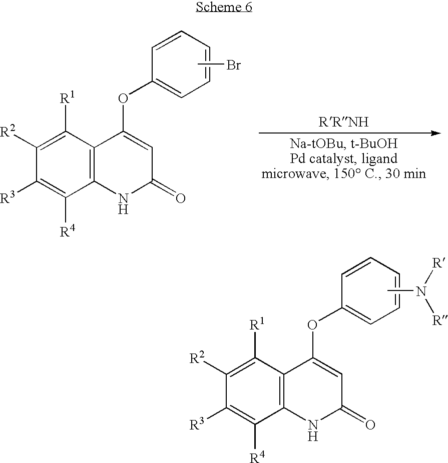

Compounds of this invention may be prepared according to Scheme 6 by reacting the bromides with the amines in the presence of base (such as potassium tert-butoxide), a solvent (such as tert-butanol), Pd catalyst (such as tris(dibenzylideneacetone)dipalladium(0) [Pd₂(dba)₃) and a ligand (such as 2-(dicyclohexylphosphino)-2′,4′,6′-tri-1-propyl-1,1′-biphenyl (X-Phos)) under microwave irradiation at 150° C. for 30 minutes.

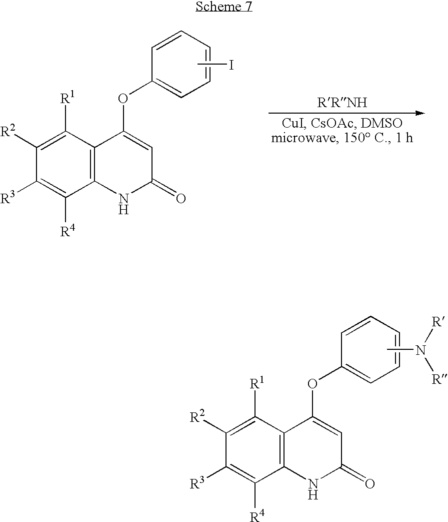

Compounds of this invention may be prepared according to Scheme 7 by reacting the iodides with the amines in the presence of copper (I) iodide, cesium acetate and DMSO under microwave irradiation at 150° C. for 1 hour.

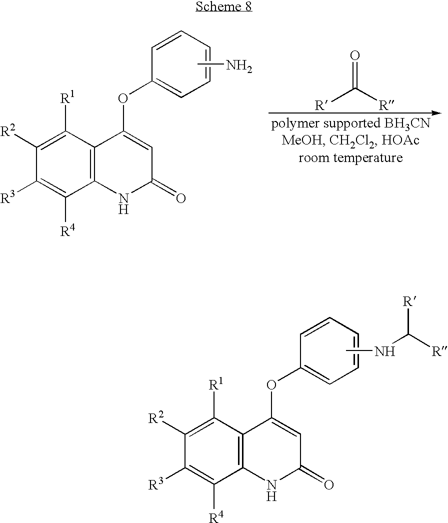

Compounds of this invention may be prepared according to Scheme 8 by reacting the amines in methanol, methylene chloride, and acetic acid with aldehydes or ketones in the presence of polymer-supported cyanoborohydride at room temperature.

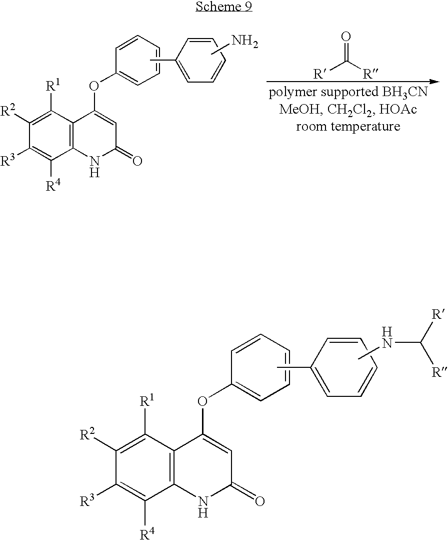

Compounds of this invention may be prepared according to Scheme 9 by reacting the amines in methanol, methylene chloride, and acetic acid with aldehydes or ketones in the presence of polymer-supported cyanoborohydride at room temperature.

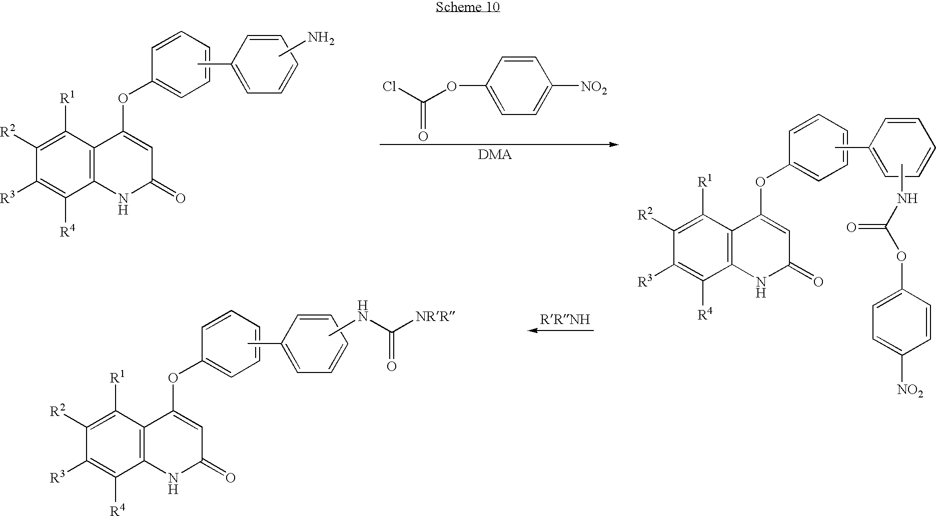

Compounds of this invention may be prepared according to Scheme 10 by first converting the amines to the p-nitrophenylcarbamate compounds, which were then reacted with amines to yield the ureas.

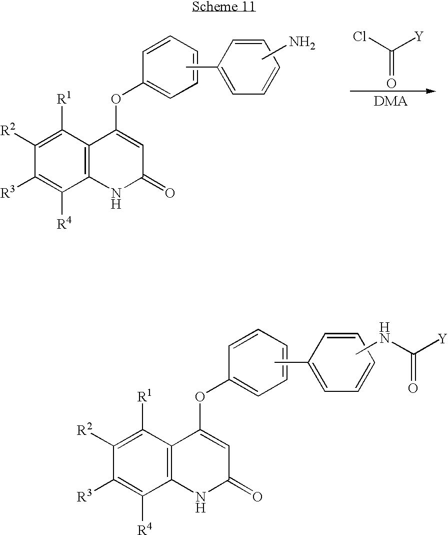

Compounds of this invention may be prepared according to Scheme 11 by reaction of the amines with acid chlorides in N,N-dimethylacetamide at 0° C., followed by warming up to room temperature.

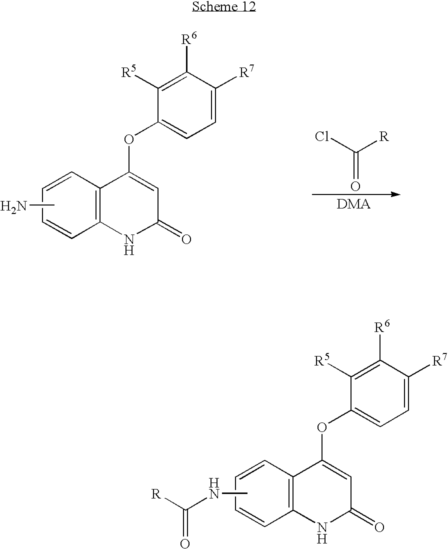

Compounds of this invention may be prepared according to Scheme 12 by reaction of the amines with acid chloride in N,N-dimethylacetamide at 0° C., followed by warming up to room temperature.

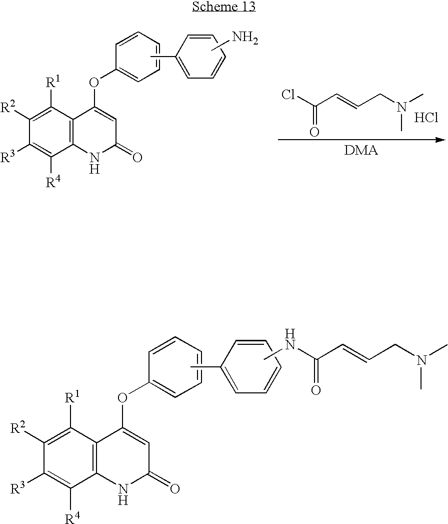

Compounds of this invention may be prepared according to Scheme 13 by first reacting the amino acid with oxalyl chloride to generate the corresponding acid chloride hydrochloride salt, and then further reacted with the amines in N,N-dimethylacetamide at 0° C.

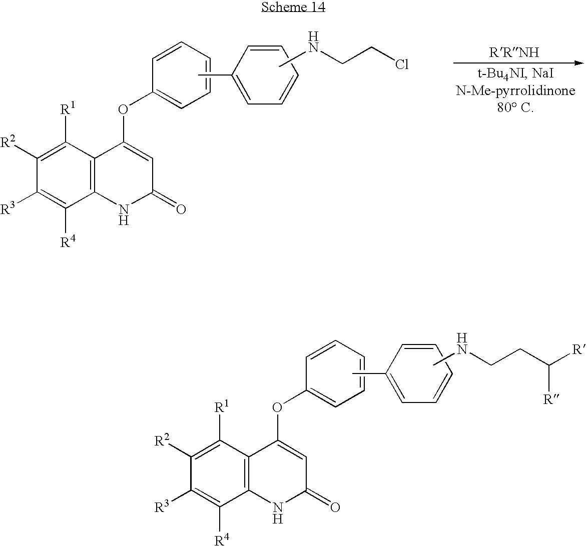

Compounds of this invention may be prepared according to Scheme 14 by reacting the chlorides with amines in the presence of tert-butylammonium iodide, sodium iodide, and N-Me-pyrrolidinone at 80° C. for 3 hours.

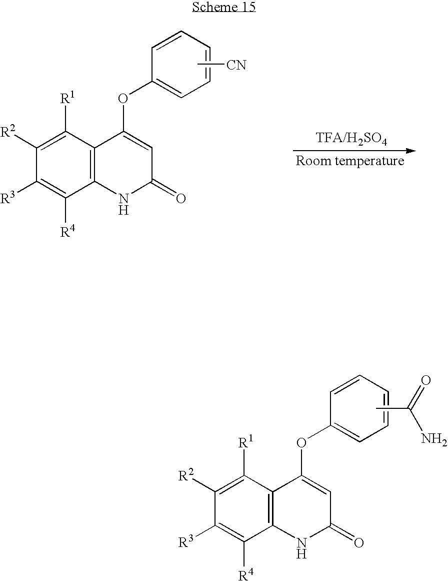

Compounds of this invention may be prepared according to Scheme 15 by hydrolyzing the nitriles in a solution of 4:1 (v:v) of trifluoroacetic acid and concentrated sulfuric acid at room temperature.

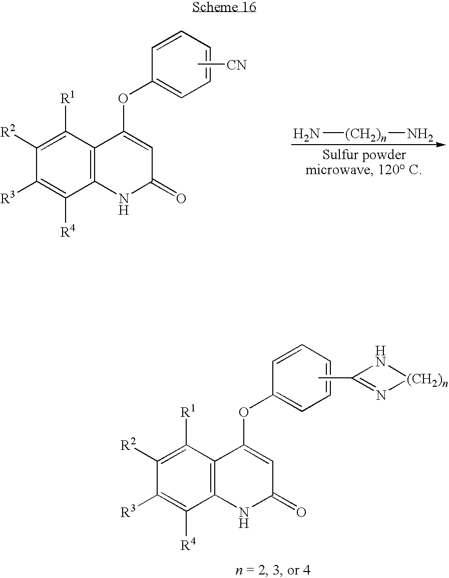

Compounds of this invention may be prepared according to Scheme 16 by reacting the nitriles with diamines and sulfur powder under microwave irradiation at 120° C. for 10 minutes.

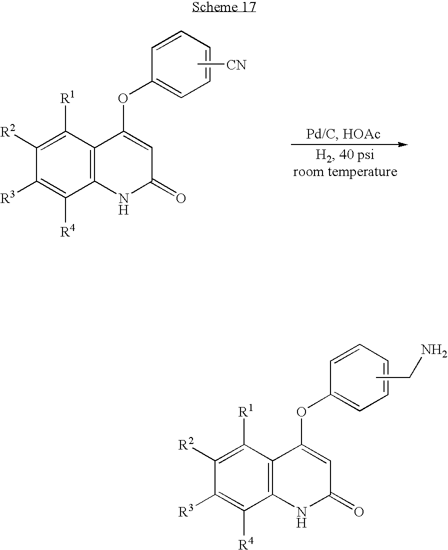

Compounds of this invention may be prepared according to Scheme 17 by reduction of nitriles with Pd/C in acetic acid under 40 psi atmosphere of hydrogen.

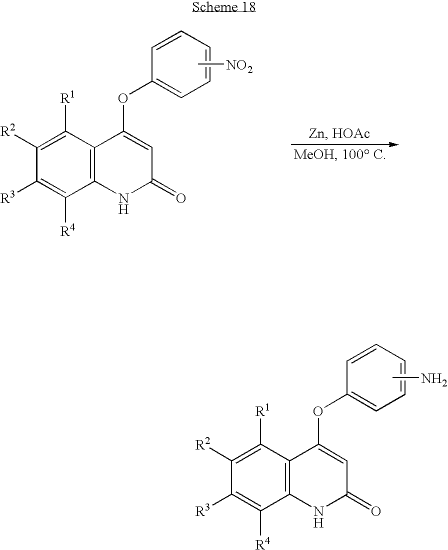

Compounds of this invention may be prepared according to Scheme 18 by reduction of the nitro compounds with zinc in acetic acid and methanol at 100° C.

### Synthesis of Intermediate Compounds

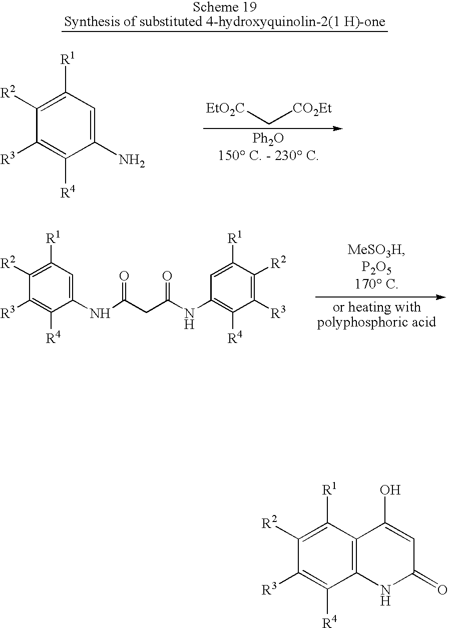

The synthesis was carried out using the procedures described in J. Heterocyclic Chem., 25, 857 (1988) [R¹═R⁴═Cl; R²═R⁴═Cl; R³═Cl, R⁴═OMe] or Bioorg. Med. Chem. Lett. 12, 811-815 (2002) [R²═NO₂].

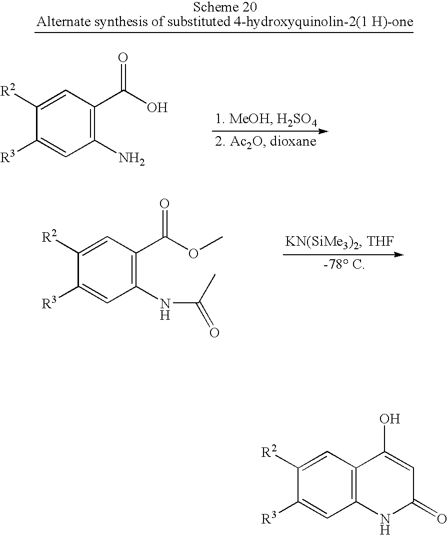

The synthesis was carried out using the procedures described in J. Med. Chem. 47 (24), 5923-36 (2004).

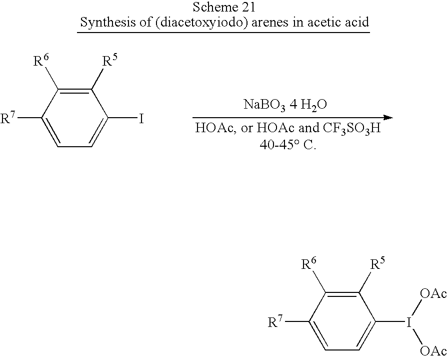

The synthesis was carried out using the procedure described in Tetrahedron 45, 3299-06 (1989) in acetic acid or the procedure described in J. Org. Chem., 70, 6984-6 (2005) in acetic acid and trifluorosulfonic acid.

One of skill in the art will recognize that Schemes 1-21 can be adapted to produce the other compounds of Formulas I and pharmaceutically acceptable salts of compounds of Formulas I according to the present invention.

### EXAMPLES

The following abbreviations are used herein and have the indicated definitions: ACN is acetonitrile, AcOH is acetic acid. ATP is adenosine triphosphate. βME is 2-mercaptoethanol, BOC is t-butoxycarbonyl and t-BuOH is tert-butyl alcohol or 2-methyl-2-propanol. BSA is Bovine Serum Albumin. Celite™ is flux-calcined diatomaceous earth. Celite™ is a registered trademark of World Minerals Inc. CHAPS is (3-[(3-cholamidopropyl)dimethylammonio]-1-propanesulfonic acid, DEAD is diethyl azodicarboxylate, DIAD is diisopropylazodicarboxylate, DMAP is dimethyl aminopyridine, DME is 1,2-dimethoxyethane, DMF is N,N-dimethylformamide, DMF-DMA is dimethylformamide dimethyl acetal, and DMSO is dimethylsulfoxide. DPBS is Dulbecco's Phosphate Buffered Saline Formulation, DTT is (2S,3S)-1,4-bis-sulfanylbutane-2,3-diol or dithiothreitol, EDTA is ethylenediaminetetraacetic acid, EGTA is ethylene glycol tetraacetic acid, ESI stands for Electrospray Ionization, EtOAc is ethyl acetate, and EtOH is ethanol. FLAG-TOR is a FLAG-tagged TOR protein, HBTU is O-benzotriazole-N,N,N′,N′-tetramethyl-uronium-hexafluoro-phosphate, HEPES is 4-(2-hydroxyethyl)-1-piperazineethanesulfonic acid, GMF is glass microfiber, HOBT is N-hydroxybenzotriazole, Hunig's Base is diisopropylethylamine, HPLC is high-pressure liquid chromatography, LPS is lipopolysaccharide. MeCN is acetonitrile, MeOH is methanol, microcrystin LR is a cyclic heptapeptide hepatotoxin produced Microcystis aeruginosa containing the amino acids leucine (L) and arginine (R) in the variable positions, MS is mass spectrometry, mTOR is Mammalian Target of Rapamycin (a protein), and NEt₃ is triethylamine. PI3K is phosphoinositide 3-kinase (an enzyme). NMP is N-methylpyrrolidone, NMR is nuclear magnetic resonance, PBS is phosphate-buffered saline (pH 7.4), RPMI 1640 is a buffer (Sigma-Aldrich Corp., St. Louis, Mo., USA), SDS is dodecyl sulfate (sodium salt), SRB is Sulforhodamine B, TCA is trichloroacetic acid, TFA is trifluoroacetic acid, THF is tetrahydrofuran, THP is tetrahydro-2H-pyran-2-yl. TLC is thin-layer chromatography and TRIS is tris(hydroxymethyl)aminomethane.

### Synthetic Methods

### Procedure 1:

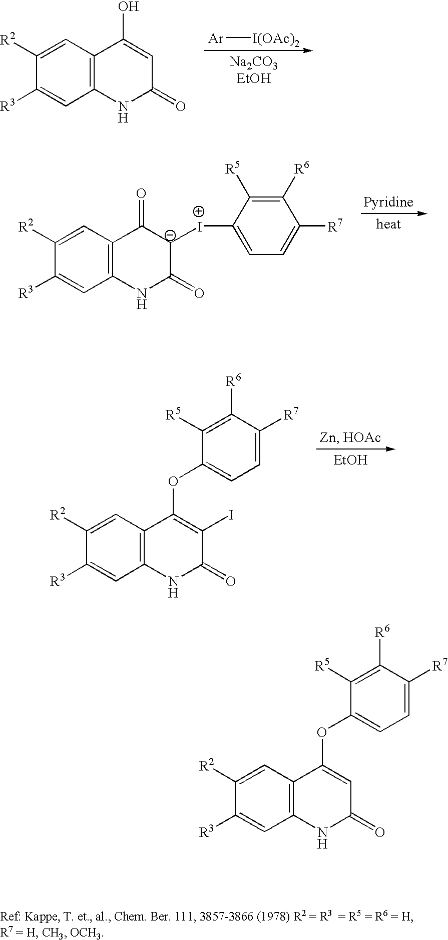

### Example 2

### Synthesis of 6-Chloro-4-phenoxyquinolin-2(1H)-one

To a solution of 6-chloro-4-hydroxyquinolin-2(1H)-one (200 mg, 1.022 mmol) and aqueous sodium carbonate (0.1 N, 10 ml) at 38° C. was added a suspension of diacetoxyiodobenze (330 mg, 1.022 mmol), aqueous sodium carbonate (0.1 N, 10 ml), and a small amount of ethanol. After heating at 38° C. for 1 hour 20 minutes, it was cooled and filtered, washed with water to give 324 mg of 6-chloro-2,4-dioxo-3-(phenyl)odonio)-1,2,3,4-tetrahydroquinolin-3-ide as a pink solid (80% yield).

6-Chloro-2,4-dioxo-3-(phenyl)odonio)-1,2,3,4-tetrahydroquinolin-3-ide (319 mg, 0.8 mmol) was heated with pyridine (10 ml) at 118° C. for 2 hours. The solution was cooled and treated with ice water. The solid was filtered and washed with water to give 269 mg of 6-chloro-3-iodo-4-phenoxyquinolin-2(1H)-one as a white solid (85% yield).

6-Chloro-3-iodo-4-phenoxyquinolin-2(1H)-one (39.7 mg, 0.1 mmol) was treated with acetic acid (1 ml), ethanol (1 ml), and zinc dust (34 mg, 0.52 mmol). After the mixture was heated for 3.5 hours, it was filtered and washed with ethanol to yield 21.7 mg of 6-chloro-4-phenoxyquinolin-2(1H)-one as a white solid (80% yield).

The following examples were prepared according to the procedure of Example 2:

| | | | | | | Yield |
|---|---|---|---|---|---|---|
| Ex. # | R² | R³ | R⁵ | R⁶ | R⁷ | (%) |
| 1 | H | H | H | H | H | 62 |
| 2 | Cl | H | H | H | H | 80 |
| 3 | H | H | H | OCH₃ | H | 81 |
| 4 | H | H | H | H | OCH₃ | 74 |
| 5 | H | H | H | H | CH₃ | 89 |
| 6 | H | H | H | CH₃ | H | 99 |
| 7 | H | H | H | H | F | 94 |
| 8 | H | H | H | H | NH₂ | 50 |
| 9 | H | H | H | H | NAc | 8 |
| 10 | H | Cl | H | H | H | 92 |
| 17 | H | Br | H | H | H | 97 |
| 19 | H | H | OCH₃ | H | H | 78 |
| 20 | H | H | CH₃ | H | H | 79 |
| 21 | H | H | H | H | Br | 65 |
| 31 | H | H | H | H | Ac | 66 |
| 32 | H | H | H | H | CN | 66 |
| 33 | H | H | H | H | Cl | 62 |
| 25 | H | H | H | Br | H | 92 |
| 35 | H | H | H | CF₃ | H | 95 |
| 36 | H | H | H | H | OCF₃ | 83 |
| 39 | H | H | H | CH₂CN | H | 93 |
| 37 | H | H | Br | H | H | 98 |
| 38 | H | H | H | OCF₃ | H | 100 |
| 40 | H | H | H | H | OCF₃ | 100 |
| 11 | Br | H | H | H | H | 87 |
| 16 | NH₂ | H | H | H | H | 61 |
| 21 | H | H | H | H | Br | 56 |
| 46 | H | H | H | CH₃ | CH₃ | 20 |

### Procedure 2:

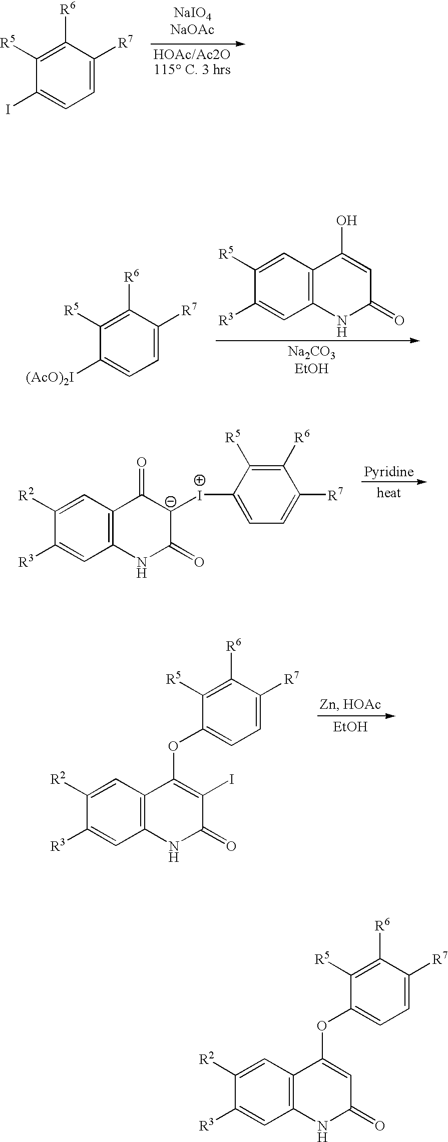

### Example 58

### Synthesis of 4-[(4-difluoromethoxy)phenoxy]quinolin-2(1H)-one

A mixture of 4-(difluoromethoxy)phenol (2.70 g, 10.0 mmol), sodium acetate (1.8 g, 22.0 mmol), sodium periodate (2.25 g, 10.5 mmol), acetic acid (15 ml) and acetic anhydride (1.5 ml) was stirred and heated at 115° C. for 3 hours. The reaction was then cooled to room temperature, solvents evaporated and the residue treated with water, filtered, washed with 10% acetic acid in water, water then ethyl acetate and dried to give 2.172 g of the diacetoxyiodo-4-difloromethoxybenzene (56% yield).

To a solution of 4-hydroxyquinolin-2(1H)-one (902 mg, 5.6 mmol) and aqueous sodium carbonate (0.1 N, 56 ml) at 38° C. was added a suspension of the diacetoxyiodo-4-difloromethoxybenzene (2.172 g, 5.6 mmol), aqueous sodium carbonate (0.1 N, 56 ml), and a small amount of ethanol. After heating at 38° C. for 1 hour 20 minutes, it was cooled and filtered, washed with water to give a solid, which was dried, then heated with pyridine (50 ml) at 118° C. for 2 hours. The solution was cooled and treated with ice water. The solid was filtered and washed with water to give 858 mg of 3-iodo-4-phenoxyquinolin-2(1H)-one as a white solid (36% yield). This was treated with acetic acid (28 ml), ethanol (28 ml), and zinc dust (520 mg, 7.95 mmol). After the mixture was heated for 3.5 hours, it was filtered and washed with ethanol, the filtrate was evaporated, treated with water, the solids collected and dried to yield 548 mg of 4-(4-(difluoromethoxy)phenoxy)quinolin-2(1H)-one as a white solid (90% yield).

The following examples were prepared according to the procedure of Example 58.

| Ex. # | R² | R³ | R⁵ | R⁶ | R⁷ | Yield (%) |
|---|---|---|---|---|---|---|
| 58 | H | H | H | H | OCHF₂ | 90 |
| 59 | H | H | H | H | OEt | 83 |
| 77 | H | H | H | F | H | 31 |
| 78 | H | H | H | CO₂Me | OMe | 72 |
| 79 | H | H | Benzo | | H | 79 |
| 80 | H | H | CF₃ | H | H | 57 |

### Procedure 3:

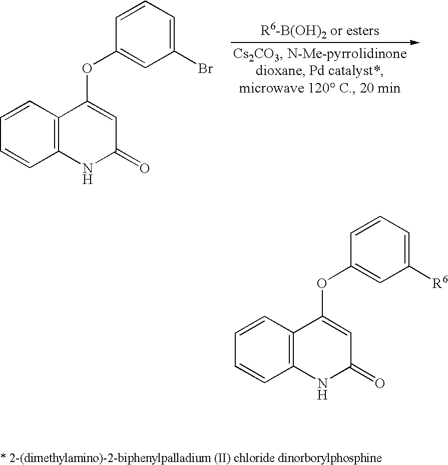

### Example 41

### Synthesis of 4-(3-pyridin-3-ylphenoxy)quinolin-2(1H)-one

A mixture of 4-(3-bromophenoxy)quinolin-2(1H)-one (31.6 mg, 0.1 mmol), (3-pyridinyl)boronic acid (25 mg, 0.2 mmol), 2-(dimethylamino)-2-biphenylpalladium (II) chloride dinorborylphosphine (8.4 mg, 0.015 mmol), cesium carbonate (82 mg, 0.25 mmol), N-Me-pyrrolidinone (1.1 ml) and dioxane (1.1 ml) was heated at 120° C. in a microwave for 20 minutes. After it was filtered through a pad of Celite™, the filtrate was dried up and washed with water to yield 29 mg of 4-(3-pyridin-3-ylphenoxy)quinolin-2(1H)-one as a white solid (92% yield).

The following examples were prepared according to the procedure of Example 41:

| Ex. # | R⁶ | Yield (%) |
|---|---|---|
| 41 | 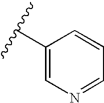 | 92 |
| 42 | 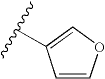 | 36 |
| 43 | 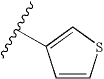 | 56 |
| 55 | 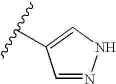 | 1.3 |
| 54 | 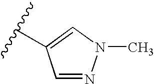 | 17 |
| 56 | 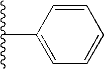 | 23 |
| 60 | 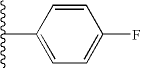 | 11 |

### Procedure 4:

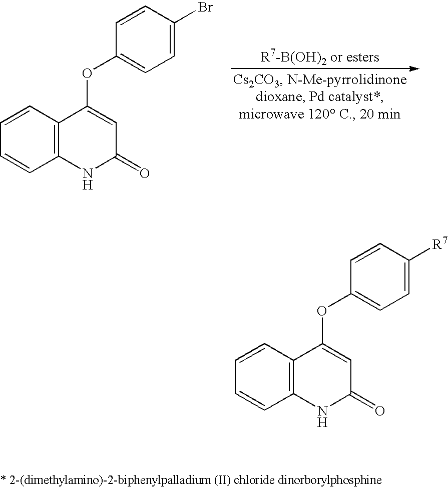

The following examples were prepared according to the procedure of Example 41, starting with a 4-bromophenoxy compound:

| Ex. # | R⁷ | Yield (%) |
|---|---|---|
| 26 | 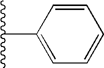 | 53 |
| 27 | 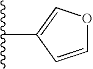 | 34 |
| 28 | 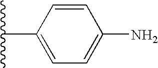 | 60 |
| 29 | 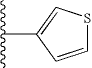 | 79 |
| 30 | 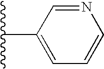 | 66 |
| 34 | 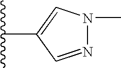 | 53 |
| 47 | 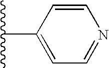 | 67 |
| 48 | 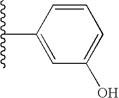 | 58 |

### Procedure 5:

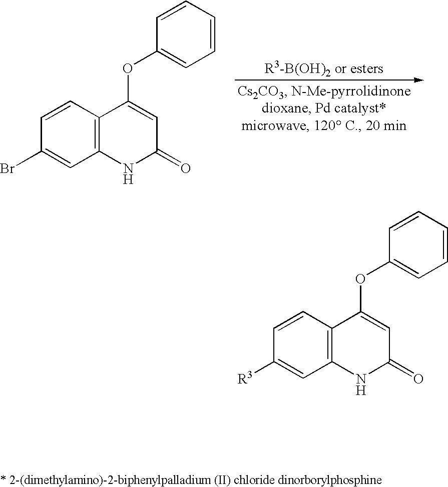

### Example 53

### Synthesis of 7-(1-methyl-1H-pyrazol-4-yl)-4-phenoxyquinolin-2(1H)-one

A mixture of 7-bromo-4-phenoxyquinolin-2(1H)-one (63.2 mg, 0.2 mmol), 1-methylpyrazole-4-boronic acid pinacol ester (84 mg, 0.4 mmol), 2-(dimethylamino)-2-biphenylpalladium (II) chloride dinorborylphosphine (16.8 mg, 0.03 mmol), cesium carbonate (164 mg, 0.5 mmol), N-Me-pyrrolidinone (2.2 ml) and dioxane (2.2 ml) was heated at 120° C. in a microwave for 20 minutes. After it was filtered through a pad of Celite™, the filtrate was dried up and purified by C-18 reverse-phase HPLC to yield 8.4 mg of 7-(1-methyl-1H-pyrazol-4-yl)-4-phenoxyquinolin-2(1H)-one as a white solid (13% yield).

The following examples were prepared according to the procedure of Example 53:

| Ex. # | R³ | Yield (%) |
|---|---|---|
| 52 | 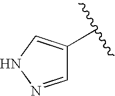 | 2.4 |
| 53 | 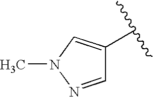 | 13 |

### Procedure 6:

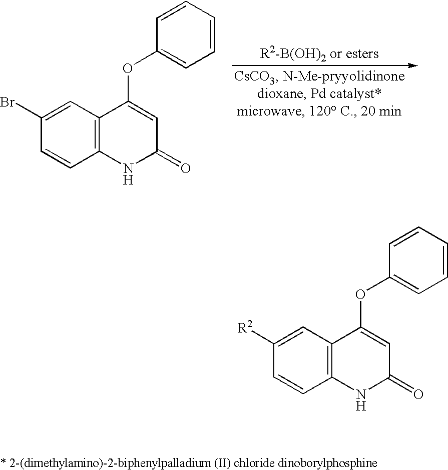

The following examples were prepared according to the procedure of Example 53, starting with a 6-bromoquinoline compound:

| Ex. # | R² | Yield (%) |
|---|---|---|
| 12 | 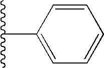 | 40 |
| 13 | 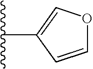 | 26 |

### Procedure 7:

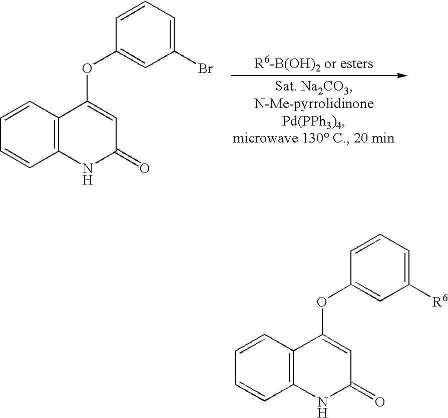

### Example 61

### Synthesis of 4-[(4′-aminobiphenyl-3-yl)oxy]quinolin-2(1H)-one

A mixture of 4-(3-bromophenoxy)quinolin-2(1H)-one (63 mg, 0.2 mmol), tetrakis(triphenylphosphine)palladium(0) (35 mg, 0.03 mmol), saturated sodium carbonate (0.5 ml), N-Me-pyrrolidinone (2 ml) and 4-(4,4,5,5-tetramethyl-1,3,2-dioxaborolan-2-yl)aniline (88 mg, 0.4 mmol) was heated at 130° C. in a microwave for 20 minutes. It was filtered through a pad of Celite™ and washed with chloroform. After drying up the filtrate, the residue was treated with acetonitrile and filtered to yield 34 mg of 4-[(4′-aminobiphenyl-3-yl)oxy]quinolin-2(1H)-one as a white solid (52% yield).

The following examples were prepared according to the procedure of Example 61.

| Ex. # | R⁶ | Yield (%) |
|---|---|---|
| 61 | 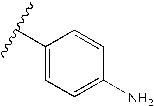 | 52 |
| 62 | 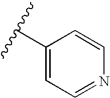 | 48 |
| 64 | 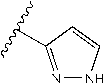 | 79 |
| 65 | 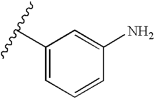 | 30 |
| 75 | 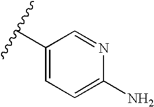 | 75 |
| 76 | 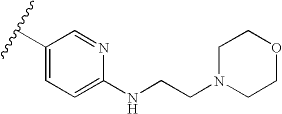 | 44 |
| 100 | 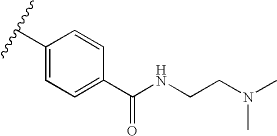 | 39 |
| 101 | 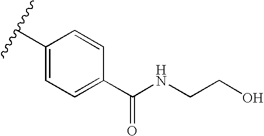 | 57 |
| 102 | 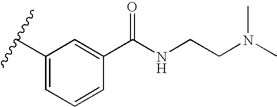 | 67 |
| 103 | 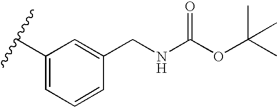 | 40 |
| 104 | 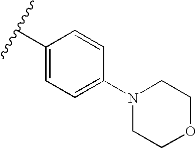 | 75 |
| 105 | 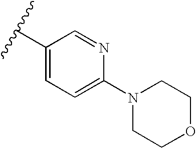 | 62 |
| 106 | 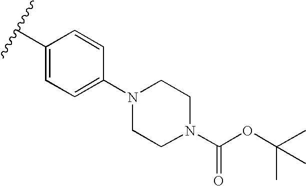 | 39 |
| 107 | 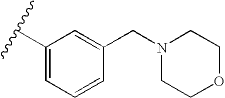 | 22 |
| 114 | 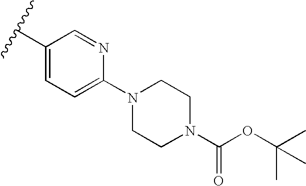 | 49 |
| 115 | 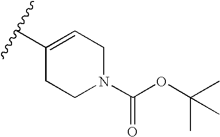 | 27 |
| 123 | 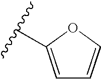 | 83 |
| 124 | 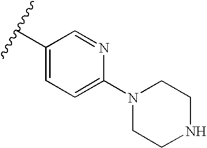 | 70 |
| 125 | 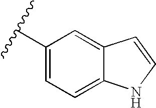 | 30 |
| 126 | 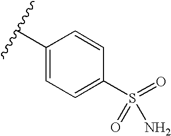 | 51 |
| 127 | 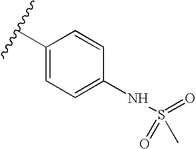 | 49 |
| 131 | 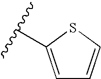 | 55 |
| 132 | 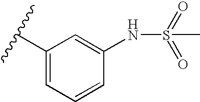 | 54 |
| 133 | 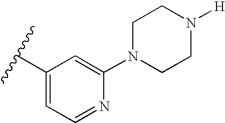 | 31 |
| 164 | 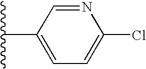 | 23 |
| 71 | 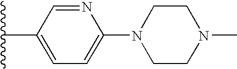 | 65 |
| 74 | 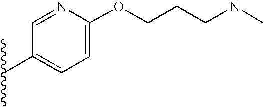 | 54 |
| 82 | 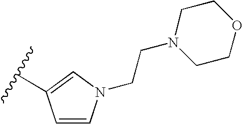 | 19 |
| 83 | 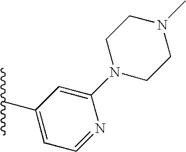 | 30 |
| 85 | 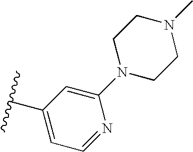 | 29 |
| 86 | 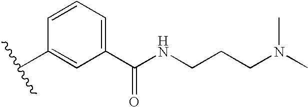 | 41 |
| 87 | 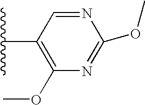 | 43 |

### Example 75

### Data for 4-[3-(6-aminopyridin-3-yl)phenoxy]quinolin-2(1H)-one

MS (ESI) m/z 330.3;

HRMS: calcd for C20H15N3O2+H⁺, 330.12370; found (ESI-FTMS, [M+H]¹⁺), 330.12425;

¹H NMR (400 MHz, DMSO-d₆) δ ppm 5.48 (s, 1H), 6.15 (s, 2H), 6.5 (d, J=8.6 Hz, 1H), 7.18 (d, J=8.3 Hz, 1H), 7.27 (t, J=6.8 Hz, 1H), 7.35 (d, J=8.1 Hz, 1H), 7.5-7.65 (m, 4H), 7.78 (dd, J=8.1, 2.6 Hz, 1H), 8 (d, J=8.6 Hz, 1H), 8.32 (d, J=1.8 Hz, 1H), 11.57 (s, 1H).

### Example 82

### Data for 4-{3-[1-(2-morpholin-4-ylethyl)-1H-pyrazol-4-yl]phenoxy}quinolin-2(1H)-one

1H NMR (400 MHz, DMSO-d₆) δ ppm 2.39-2.42 (m, 4H), 2.73 (t, J=6.4 Hz, 2H), 3.5-3.56 (m, 4H), 4.23 (t, J=6.4 Hz, 2H), 5.76 (d, J=1.4 Hz, 1H), 7.10 (dd, J=8.0, 1.4 Hz, 1H) 7.27 (t, J=8.4 Hz, 1H), 7.36 (d, J=8.4 Hz, 1H), 7.48-7.64 (m, 4H), 7.96 (s, 1H), 8.0 (d, J=8.0 Hz, 1H), 8.30 (s, 1H), 11.57 (s, 1H).

### Procedure 8:

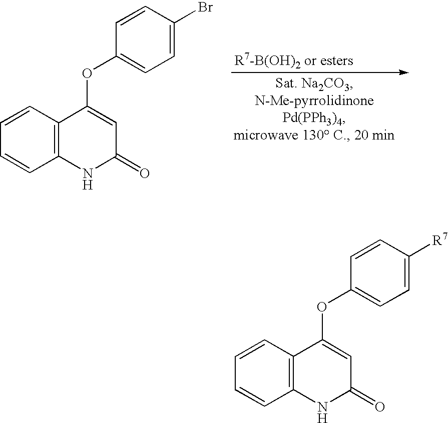

The following examples were prepared according to the procedure of Example 61.

| Ex. # | R⁷ | Yield (%) |
|---|---|---|
| 49 | 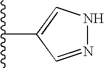 | 48 |
| 50 | 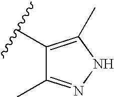 | 26 |
| 74 | 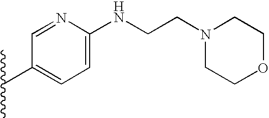 | 30 |
| 84 | 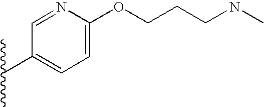 | 60 |

### Procedure 9:

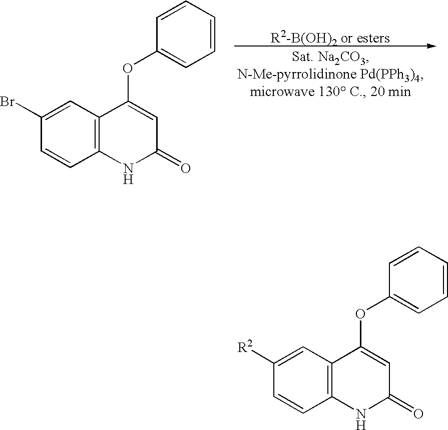

### Example 14

### Synthesis of 4-phenoxy-6-(1H-pyrazol-3-yl)quinolin-2(1H)-one

A mixture of 5-bromo-4-phenoxyquinolin-2(1H)-one (63 mg, 0.2 mmol), tetrakis(triphenylphosphine)palladium(0) (35 mg, 0.03 mmol), saturated sodium carbonate (0.5 ml), N-Me-pyrrolidinone (2 ml) and pyrazol-3-ylboronic acid (45 mg, 0.4 mmol) was heated at 130° C. in a microwave for 20 minutes. It was filtered through a pad of Celite™ and washed with chloroform. After drying up the filtrate, the residue was treated with water, filtered and washed with acetonitrile to yield 49 mg of 4-phenoxy-6-(1H-pyrazol-3-yl)quinolin-2(1H)-one as a white solid (81% yield).

The following examples were prepared according to the procedure of Example 14.

| Ex. # | R² | Yield (%) |
|---|---|---|
| 14 | 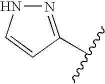 | 81 |
| 63 | 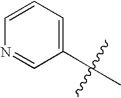 | 48 |
| 44 | 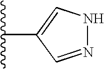 | 39 |
| 51 | 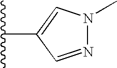 | 78 |
| 67 | 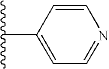 | 87 |
| 68 | 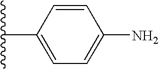 | 54 |

### Procedure 10:

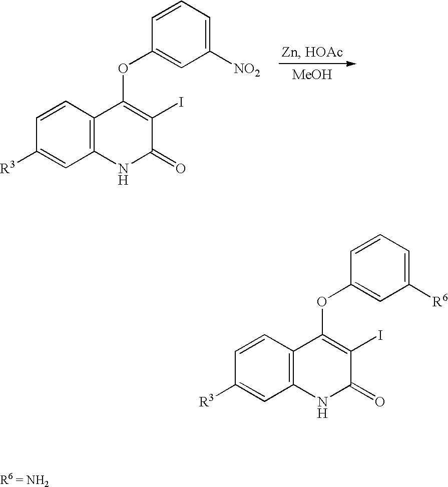

### Example 91

### Synthesis of 4-(3-aminophenoxy)-7-chloroquinolin-2(1H)-one

A mixture of 7-chloro-3-iodo-4-(3-nitrophenoxy)quinolin-2(1H)-one (0.42 g, 0.949 mmol), ethanol (9 ml), acetic acid (9 ml), and zinc dust (0.309 g, 4.745 mmol) was heated at 100° C. for 3 hours. After it was filtered, the filtrate was dried up and washed with water and ether to give 0.137 g of 4-(3-aminophenoxy)-7-chloroquinolin-2(1H)-one as a white solid (50% yield).

The following examples were prepared according to the procedure of Example 91.

| Ex. # | R⁶ | Yield (%) |
|---|---|---|
| 45 | H | 84 |
| 91 | Cl | 50 |

### Procedure 11:

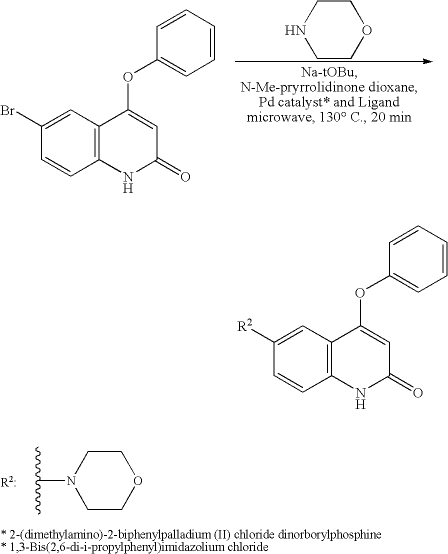

### Example 15

### 6-morpholin-4-yl-4-phenoxyquinolin-2(1H)-one

A mixture of 6-bromo-4-phenoxyquinolin-2(1H)-one (200 mg, 0.63 mmol), morpholine (0.27 ml, 3.15 mmol), 2-(dimethylamino)-2-biphenylpalladium (II) chloride dinorborylphosphine (53 mg, 0.095 mmol), 1,3-bis(2,6-di-1-propylphenyl)imidazolium chloride, potassium tert-butoxide (182 mg, 1.82 mmol), N-Me-pyrrolidinone (2.0 ml) and dioxane (2.0 ml) was heated at 130° C. in a microwave for 20 minutes. After it was filtered through a pad of Celite™, the filtrate was dried up and purified silica gel column chromatography 1:9 methanol:methylene chloride to recover 75.0 mg of example 15 as a greenish yellow solid (36%)

| Ex. # | R² | Yield (%) |
|---|---|---|
| 15 | 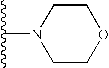 | 36 |

### Procedure 12:

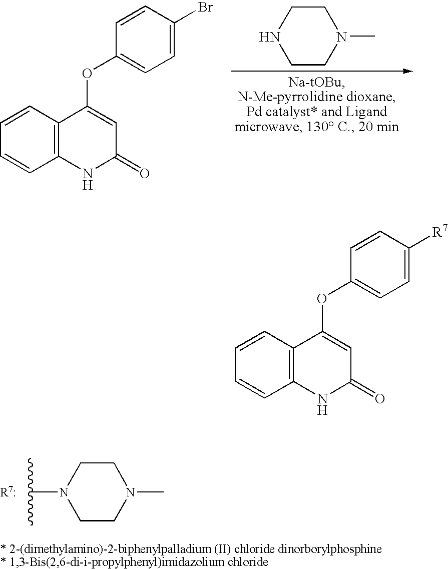

The following were prepared using the same procedure as example 15 and starting with 4-(4-bromophenoxy)quinolin-2(1H)-one.

| Ex. # | R⁷ | Yield (%) |
|---|---|---|
| 69 | 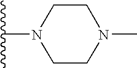 | 16 |

### Procedure 13:

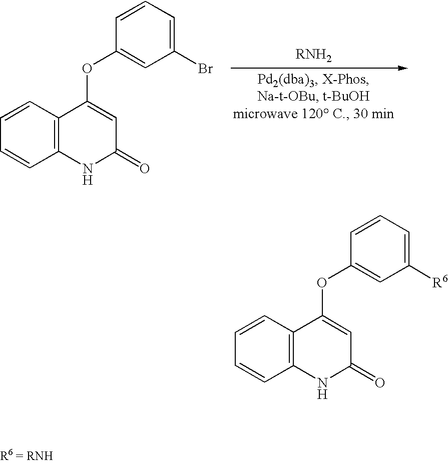

### Example 139

### Synthesis of 4-{3-[(2-pyridin-3-ylethyl)amino]phenoxy}quinolin-2(1H)-one

A mixture of 4-(3-bromophenoxy)quinolin-2(1H)-one (126 mg, 0.4 mmol), tris(dibenzylideneacetone)dipalladium (0) [Pd₂(dba)₃] (18.4 mg, 0.02 mmol), 2-(dicyclohexylphosphino)-2′,4′,6′-tri-1-propyl-1,1′-biphenyl (X-Phos) (19.1 mg, 0.04 mmol), sodium t-butoxide (54 mg, 0.56 mmol), 3-(aminoethyl)pyridine (58 μL, 0.48 mmol) and t-butanol (1 ml) was heated at 120° C. in a microwave for 30 minutes. It was filtered through a pad of Celite™ and washed with chloroform. The filtrate was dried, washed with water and acetonitrile to yield 121 mg of a crude product. Further purification on prep TLC plate (developed in 7.5% methanol in ethyl acetate) to yield 22 mg of white solid (15% yield).

| Ex. # | R⁶ | Yield (%) |
|---|---|---|
| 139 | 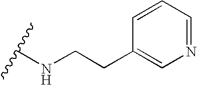 | 15 |

[symbol]

### Procedure 14:

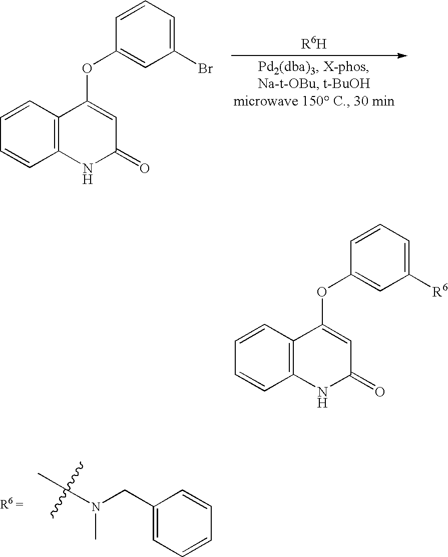

### Example 161

### Synthesis of 4-{3-[methyl(pyridin-3-ylmethyl)amino]phenoxy}quinolin-2(1H)-one

To a 5.0 ml microwave tube was added 4-(3-bromophenoxy)quinolin-2(1H)-one (316 mg, 1.0 mmol), 2-Dicyclohexylphosphino-2′,4′,6′-tri-iso-propyl-1,1′-biphenyl (95 mg, 0.200 mmol), sodium 2-methylpropan-2-olate (269 mg, 2.80 mmol), Tris(dibenzylideneacetone)dipalladium(0) (92 mg, 0.100 mmol) and t-BuOH (3.5 ml). This was sealed and then heated at 150° C. for 30 minutes in the microwave. Reaction mixture was filtered through Celite™ and washed with 10% methanol in chloroform. The resulting orange solution was evaporated. This solution was evaporated, dissolved in 15% methanol in chloroform, loaded onto a 20 cm×20 cm 2 mm prep TLC plate and eluted with 10% methanol in chloroform. The third colored band down from the solvent front was product by MS and LC/MS. This band was collected and washed with 15% methanol in chloroform, eluate evaporated and the residue treated with acetonitrile, solid collected on a sintered glass funnel, washed with acetonitrile, then air dried to give a white solid, 4-(3-(methyl(pyridin-3-ylmethyl)amino)phenoxy)quinolin-2(1H)-one (59.8 mg, 0.167 mmol, 16% yield). mp 187-93° C. dec; HRMS: calcd for C₂₂H₁₉N₃O₂+H⁺, 358.15500; found (ESI-FTMS, [M+H]¹⁺), 358.15488;

### Procedure 15:

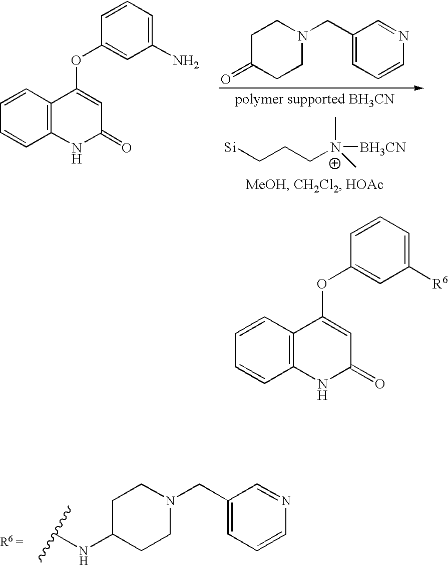

### Example 158

### Synthesis of 4-(3-{[1-(pyridin-2-ylmethyl)piperidin-4-yl]amino}phenoxy)quinolin-2(1H)-one

To a solution of 4-(3-aminophenoxy)quinolin-2(1H)-one (0.12 g, 0.48 mmol), methanol (10 ml) and methylene chloride (10 ml) was added polymer-supported cyanoborohydride (0.5 g, 0.5 mmol). After it was stirred at room temperature for 10 minutes, 1-(pyridin-2-ylmethyl)piperidin-4-one (0.2 ml, 0.96 ml) was added. After stirring at room temperature for five days, it was filtered through a pad of Celite™, and washed with chloroform. After it was dried, the residue was treated with water, filtered and washed with acetonitrile, ether, and little bit of methanolic chloroform to yield 163 mg as a beige solid (71% yield).

The following examples were prepared according to the procedure of Example 158.

| Ex. # | R⁶ | Yield % |
|---|---|---|
| 157 | 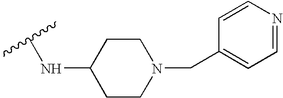 | 33 |
| 158 | 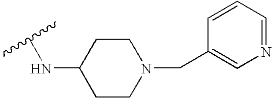 | 71 |
| 153 | 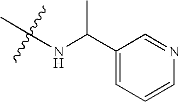 | 15 |
| 154 | 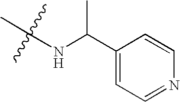 | 24 |

### Procedure 16:

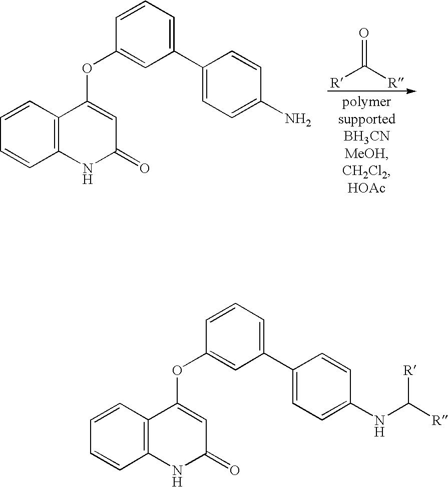

The following examples were prepared according to the procedure of Example 158.

| Ex. # | 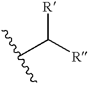 | Yield (%) |
|---|---|---|
| 116 | 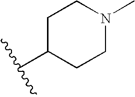 | 55 |
| 159 | 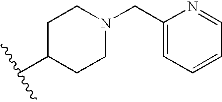 | 5 |
| 160 | 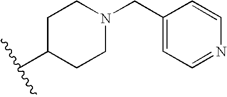 | 36 |
| 162 | 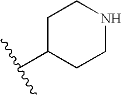 | 25 |
| 163 | 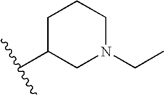 | 18 |

### Example 116

### Data for 4-({4′-[(1-methylpiperidin-4-yl)amino]biphenyl-3-yl}oxy)quinolin-2(1H)-one

1H NMR (400 MHz, DMSO-d₆) δ ppm 1.35-1.45 (m, 2H), 1.85-1.90 (m, 2H), 2.0-2.05 (m, 2H), 2.17 (s, 3H), 2.70-2.75 (m, 2H), 3.18-3.22 (m, 1H), 5.37 (s, 1H), 5.71 (d, J=8.0 Hz, 1H), 6.64 (d, J=8.4 Hz, 2H), 7.09-7.13 (m, 1H), 7.24-7.28 (m, 1H), 7.36 (d, J=8.0 Hz, 1H), 7.45-7.64 (m, 6H), 8.0 (d, J=8.4 Hz, 1H), 11.57 (s, 1H).

### Procedure 17:

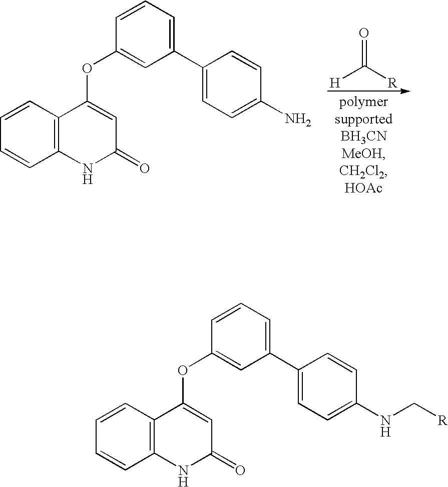

The following examples were prepared according to the procedure of Example 158.

| Ex. # | 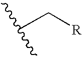 | Yield (%) |
|---|---|---|
| 108 | 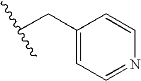 | 26 |
| 109 | 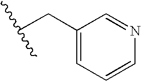 | 35 |
| 118 | 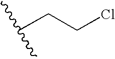 | 50 |

### Procedure 18:

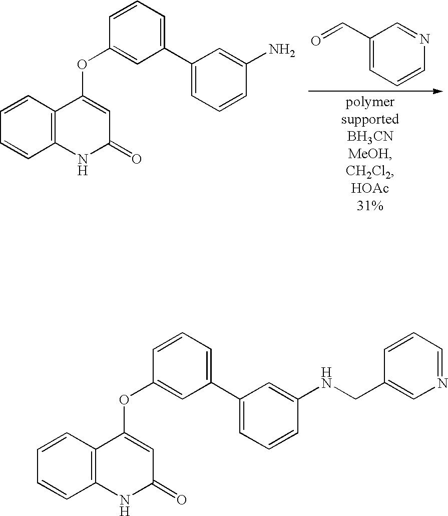

### Example 117

### Synthesis of 4-(3′-(pyridin-3-ylmethylamino)biphen-3-yloxy)quinolin-2(1H)-one

The synthesis followed the procedure of Example 158.

### Procedure 19:

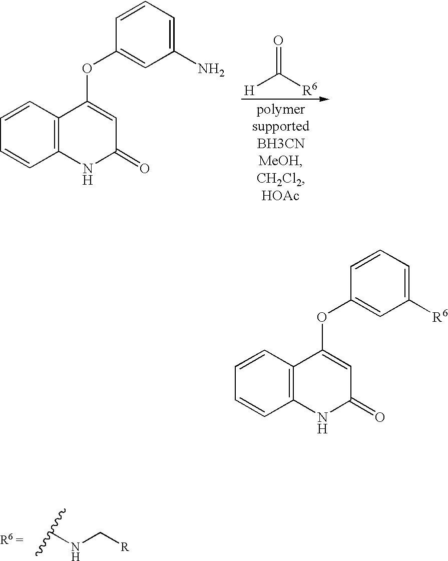

### Example 89

### Synthesis of 4-{3-[(pyridin-3-ylmethyl)-amino]-phenoxy}-1H-quinolin-2-one

To a solution of 4-(3-aminophenoxy)quinolin-2(1H)-one (0.3 g, 1.19 mmol), methanol (15 ml) and methylene chloride (15 ml) was added polymer-supported cyanoborohydride (0.5 g, 3 mmol, Aldrich silica supported ˜6 mmol/g). After it was stirred at room temperature for 10 minutes, pyridine-3-carbaldehyde (0.14 g, 0.12 ml) was added. After stirring at room temperature for 5 hours, it was filtered through a pad of Celite™, and washed with chloroform. The residue was dissolved in DMSO and purified via HPLC to afford 70 mg yellow solid (42%). 1H NMR (400 MHz, DMSO-d₆) δ ppm 4.33 (d, 2H), 5.36 (s, 1H), 6.40 (d, J=8.0 Hz, 1H), 6.46 (s, 1H), 6.59 (m, 2H), 7.21 (m, 2H), 7.35 (m, 2H), 7.58 (t, J=8.0 Hz, 1H), 7.75 (d, J=8.0 Hz, 1H), 7.92 (d, J=8.0 Hz, 1H), 8.45 (d, J=4.7 Hz, 1H), 8.58 (s, 1H), 11.58 (s, 1H).

The following examples were prepared according to the procedure of Example 89.

| Ex. # | R⁶ | Yield (%) |
|---|---|---|
| 89 | 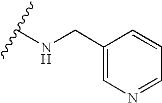 | 32 |
| 129 | 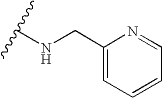 | 42 |
| 130 | 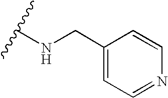 | 26 |
| 134 | 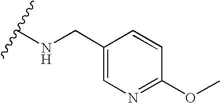 | 25 |
| 135 | 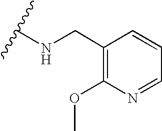 | 30 |
| 136 | 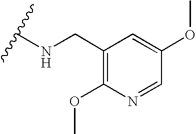 | 28 |
| 141 | 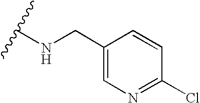 | 35 |
| 142 | 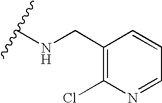 | 23 |
| 143 | 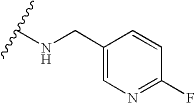 | 14 |
| 144 | 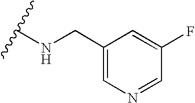 | 29 |
| 145 | 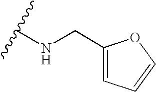 | 23 |
| 146 | 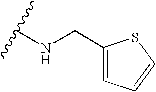 | 23 |
| 147 | 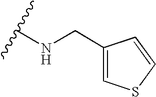 | 24 |
| 148 | 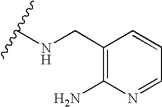 | 41 |
| 149 | 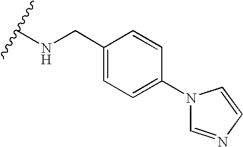 | 30 |
| 156 | 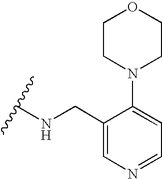 | 48 |
| 166 | 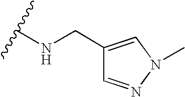 | 15 |
| 167 | 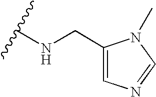 | 27 |
| 168 | 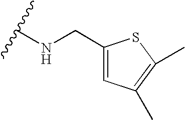 | 23 |
| 169 | 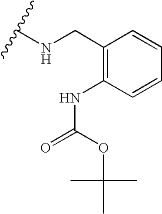 | 10 |
| 170 | 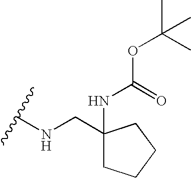 | 34 |
| 171 | 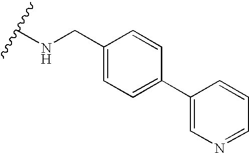 | 35 |
| 172 | 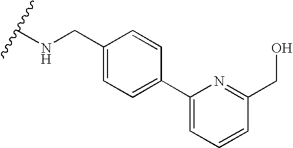 | 16 |
| 173 | 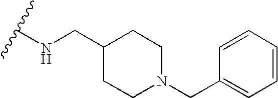 | 21 |
| 174 | 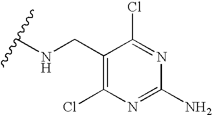 | 10 |
| 175 | 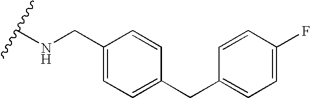 | 26 |
| 176 | 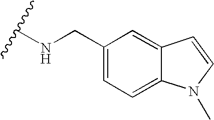 | 7 |
| 176 | 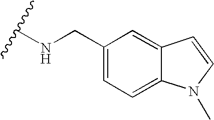 | 48 |
| 177 | 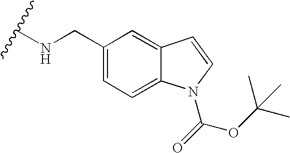 | 33 |
| 178 | 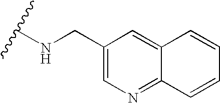 | 17 |
| 179 | 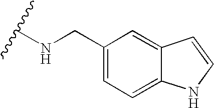 | 54 |
| 180 | 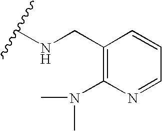 | 40 |
| 181 | 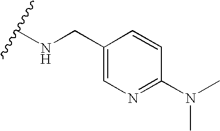 | 46 |
| 182 | 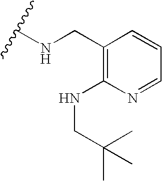 | 21 |
| 183 | 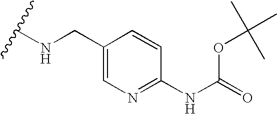 | 54 |
| 184 | 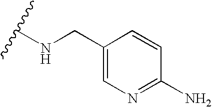 | 49 |
| 185 | 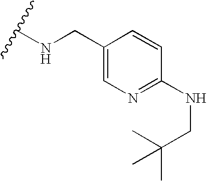 | 67 |
| 186 | 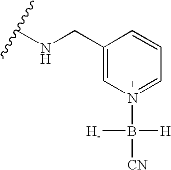 | 34 |
| 187 | 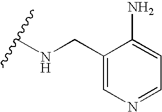 | 80 |
| 188 | 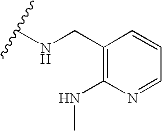 | 50 |

### Procedure 20:

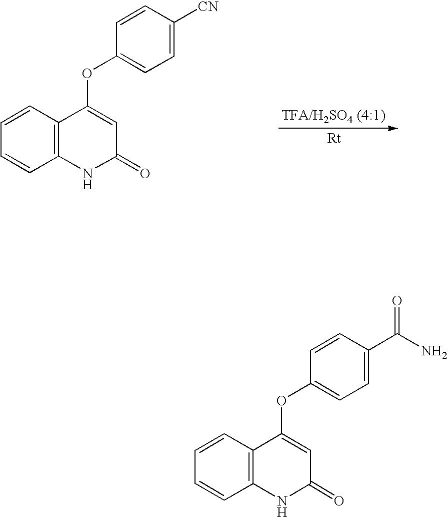

### Example 66

### Synthesis of 4-(2-oxo-1,2-dihydroquinolin-4-yloxy)benzamide

A 4:1 v:v mixture (0.4 ml) of TFA and concentrated sulfuric acid was added to 0.070 g (0.27 mmol) of 4-(2-oxo-1,2-dihydro-quinolin-4-yloxy)-benzonitrile and let react at room temperature overnight. Ice was added to the mixture and solids formed. These were filtered and rinsed with methanol and dried in vacuo to afford 25 mg (33%) of 4-(2-oxo-1,2-dihydroquinolin-4-yloxy)-benzamide as a white solid.

### Procedure 21:

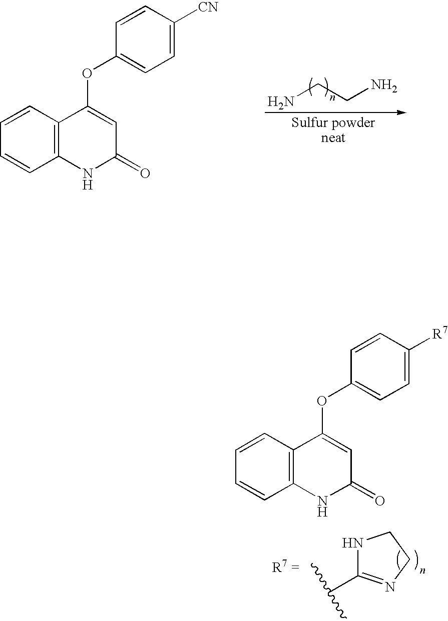

### Example 70

### Synthesis of 4-[4-(4,5-dihydro-1H-imidazol-2-yl)-phenoxy]-1H-quinolin-2-one

In a 0.5-2 mL microwave vial was combined 0.1 g (0.38 mmol) 4-(2-oxo-1,2-dihydro-quinolin-4-yloxy)-benzonitrile, 0.003 g sulfur powder (0.095 mmol), and 0.1 mL (1.53 mmol) ethane-1,2-diamine. Additional amine was added to ease stirring. The suspension was irradiated for 10 minutes at 120 deg C. Water was added and solids formed which were filtered, rinsed with diethyl ether, and dried in vacuo to afford 90 mg (77%) of 4-[4-(4,5-Dihydro-1H-imidazol-2-yl)-phenoxy]-1H-quinolin-2-one as a white solid.

The following examples were prepared according to the procedure of Example 70.

| Ex. # | R⁷ | Yield (%) |
|---|---|---|
| 70 | 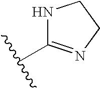 | 77 |
| 73 | 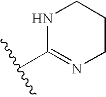 | 94 |

### Procedure 22:

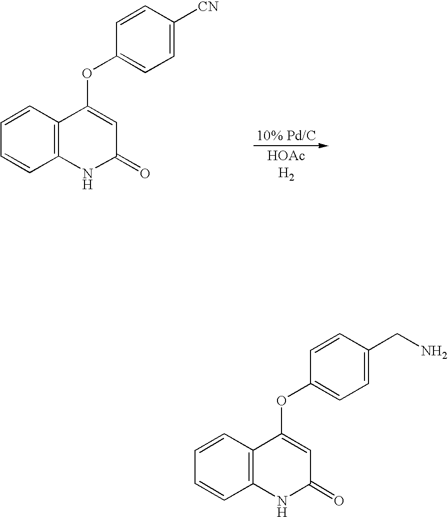

### Example 72

### Synthesis of 4-(4-aminomethyl-phenoxy)-1H-quinolin-2-one

To 0.1 g (0.38 mmol) of 4-(2-oxo-1,2-dihydro-quinolin-4-yloxy)-benzonitrile was added 5 mL glacial acetic acid, 0.040 g (0.038 mmol) of 10% Pd/C and placed on the hydrogenation apparatus with 40 psi of hydrogen and let react overnight. Filtered the suspension through Celite™ and rinsed with acetic acid. Concentrated, added water and filtered solids, which were then purified via HPLC to afford 8 mg (10%) 4-(4-Aminomethyl-phenoxy)-1H-quinolin-2-one as a white solid.

### Procedure 23:

### Alternate Synthesis of substituted 4-aryloxyquinolin-2(1H)-one

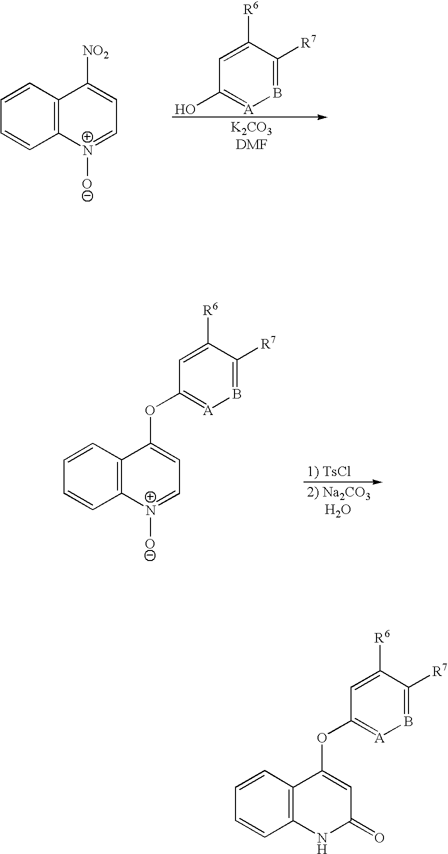

### Example 57

### Synthesis of 4-[3-(benzyloxy)phenoxy]quinolin-2(1H)-one

To stirred mixture of 3-benzyloxyphenol (2.00 g, 10 mmole), potassium carbonate (4.15 g, 30 mmole) and 25 ml DMF was added 4-nitroquinoline-N-oxide (2.09 g, 11 mmole). This was stirred and heated to a gentle reflux and kept there overnight. The reaction mixture was cooled to room temperature and quenched into 250 ml water. The solid that formed was collected by filtration, washed with water, and dried to give a beige solid, 2.79 g. A mixture of this N-oxide (250 mg, 0.73 mmole), p-tosyl chloride (167 mg, 0.87 mmole, 1.2 eq) and dichloroethane, 10 ml, was stirred and heated to reflux. This mixture was stirred for 1 hour, cooled, 10% sodium carbonate solution was added and stirred overnight. The reaction mixture was filtered, the solid was washed with water, and dried to give the product. (if no solid is present the mixture is portioned between water and ether, the ether layer was dried over sodium sulfate, filtered, evaporated, and the residue chromatographed on silica gel using a chloroform to 15% methanol in chloroform gradient, fractions containing product are combined, evaporated to give product) 149 mg (60%), mp 262-5° C.; HRMS: calcd for C₂₂H₁₇NO₃+H⁺, 344.12812; found (ESI-FTMS, [M+H]¹⁺), 344.12832.

The following examples were prepared according to the procedure of Example 57.

| Ex. # | A | B | R⁶ | R⁷ | Yield (%) |
|---|---|---|---|---|---|
| 57 | CH | CH | 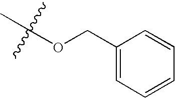 | H | 60 |
| 88 | CH | CH | 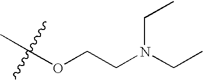 | H | 16 |
| 90 | CH | CH | H | 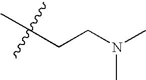 | 22 |
| 92 | CH | CH | H | I | 69 |
| 93 | CH | CH | I | H | 55 |
| 110 | CH | CH | NO₂ | H | 11 |
| 111 | CH | CH | 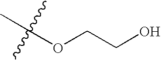 | H | 48 |
| 112 | CH | CH | 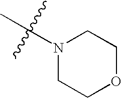 | H | 21 |
| 113 | CH | CH | —OCH₂O— | | 46 |
| 119 | CH | N | H | H | 47 |
| 137 | N | CH | H | H | 27 |

### Example 119

### Data for 4-(pyridin-3-yloxy)quinolin-2(1H)-one

mp 242-3° C.; MS (ESI) m/z 237.1; HRMS: calcd for C₁₄H₁₀N₂O₂+H⁺, 239.08150; found (ESI-FTMS, [M+H]¹⁺), 239.08141; NMR (400 MHz, DMSO d₆) δ ppm 5.36 (s, 1H), 7.27 (t, J=7.7 Hz, 1H), 7.37 (d, J=8.1 Hz), 7.59 (dd, J=8.4, 4.7 Hz, 1H), 7.62 (t, J=7.8 Hz, 1H), 7.84 (ddd, J=8.3, 2.8, 1.4 Hz, 1H), 7.98 (dd, J=8.1, 1.1, 1H), 8.59 (dd, J=4.8, 1.2 Hz, 1H), 8.63 (d, J=2.7 Hz, 1H), 11.66 (s, 1H, H-1).

### Example 137

### Data for 4-(pyridin-2-yloxy)quinolin-2(1H)-one

mp>360° C.; MS (ESI) m/z 239.1; MS (ESI) m/z 477.2; MS (ESI) m/z 302.1; HRMS: calcd for C₁₄H₁₀N₂O₂+H⁺, 239.08150; found (ESI-FTMS, [M+H]¹⁺), 239.08122; NMR (400 MHz, DMSO d₆) δ ppm 6.41 (t, J=6.6 Hz, 1H), 6.55 (d, J=9.0 Hz, 1H), 7.37 (s, 1H) 7.01 (d, J=8.0 Hz, 1H), 7.17 (t, J=7.6 Hz, 1H), 7.42 (d, J=8.3, 1H), 7.58 (t, J=7.7, 1H), 7.64 (ddd, J=9.1, 6.8, 2.0 Hz, 1H), 7.69 (d, J=6.8 Hz, 1H), 12.12 (s, 1H, H-1).

### Procedure 24:

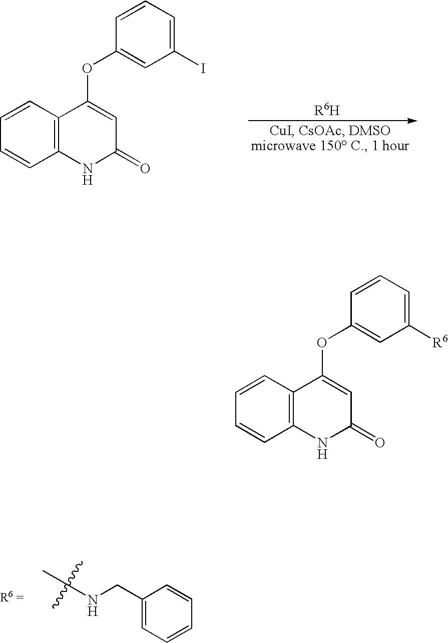

### Example 138

### Synthesis of 4-(3-(benzylamino)phenoxy)quinolin-2(1H)-one

To a 5.0 ml microwave tube was added 4-(3-iodophenoxy)quinolin-2(1H)-one (182 mg, 0.501 mmol), benzylamine (214 mg, 2.00 mmol), cesium acetate (673 mg, 3.51 mmol), copper(I) iodide (143 mg, 0.752 mmol) and DMSO (3.5 ml). This was sealed and then heated at 150° C. for 1 hour in the microwave. The reaction mixture was diluted with aqueous sodium bicarbonate solution (20 ml) and the solid formed was filtered, washed well with water, and dried. Solid was then washed with 15% MeOH in chloroform. The filtrate was passed through a short silica gel column eluting with 15% MeOH in chloroform, fractions containing product were combined and evaporated in-vacuo and the residue was treated with acetonitrile and the solid collected, (61 mg, 36% yield): mp 219-15° C.; MS (ESI) m/z 343.2; HRMS: calcd for C₁₆H₁₁F₂NO₃+H⁺, 343.14411; found (ESI-FTMS, [M+H]¹⁺), 343.14446.

The following examples were prepared according to the procedure of Example 138.

| Ex. # | R⁶ | Yield (%) |
|---|---|---|
| 138 | 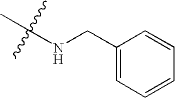 | 17 |
| 155 | 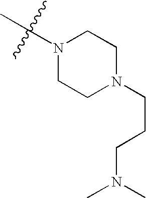 | 36 |

### Procedure 25:

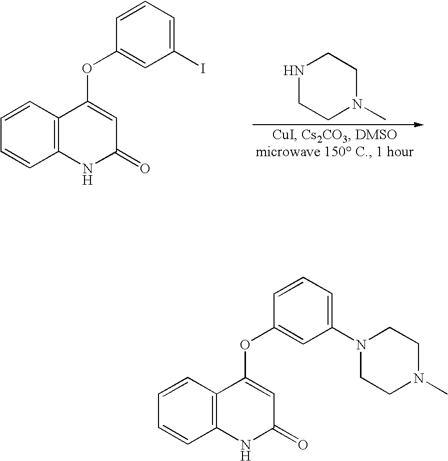

### Example 140

### Synthesis of 4-[3-(4-methylpiperazin-1-yl)phenoxy]quinolin-2(1H)-one

The above example was prepared according to the procedure of Example 138, except using cesium carbonate in place of cesium acetate, 36% yield: mp 218-21° C.; MS (ESI) m/z 336.2; HRMS: calcd for C₂₀H₂₁N₃O₂+H⁺, 336.17065; found (ESI-FTMS, [M+H]¹⁺), 336.17099.

### Procedure 26:

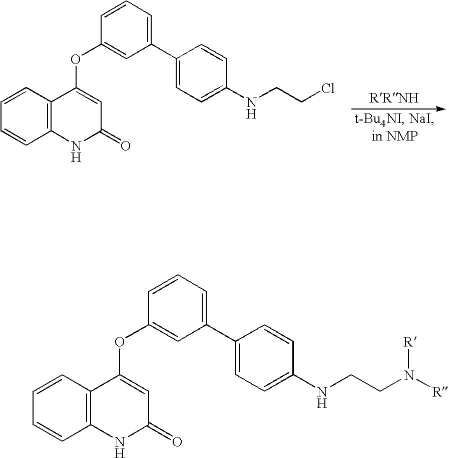

### Example 120

### Synthesis of 4-[(4′-{[2-(4-methylpiperazin-1-yl)ethyl]amino}biphenyl-3-yl)oxy]quinolin-2(1H)-one

A solution of 4-({4′-[(2-chloroethyl)amino]biphenyl-3-yl}oxy)quinolin-2(1H)-one (120 mg, 0.31 mmol), 4-metheylpiperazine (0.34 ml, 3.1 mmol), tetrabutylammonium iodide (114.8 mg, 0.31 mmol) and sodium iodide (93 mg, 0.62 mmol) in NMP was stirred at 80° C. for 3 hours. After cooling, the mixture was dissolved in chloroform (100 ml) and extracted with water, dried over sodium sulfate. The solvent was evaporated and residue was purified by thin layer chromatography (methano/methylene chloride at 15:85) to give the desired product example 120 as a yellow solid (85 mg, 61%).

The following examples were prepared according to the procedure of Example 120.

| Ex. # | 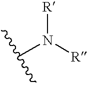 | Yield (%) |
|---|---|---|
| 120 | 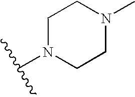 | 61 |
| 121 | 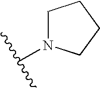 | 55 |
| 122 | 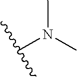 | 39 |
| 151 | 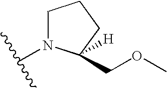 | 43 |

### Procedure 27:

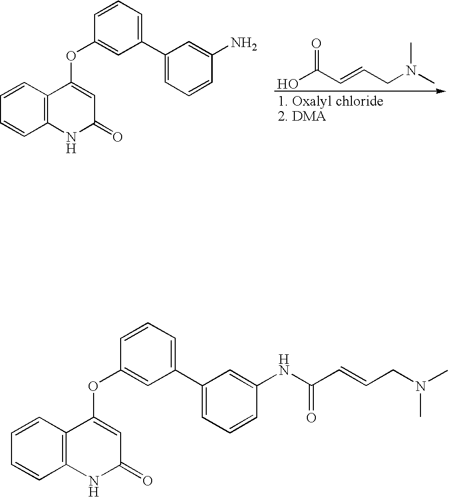

### Example 152

### Synthesis of (2E)-4-(di methylamino)-N-{3′-[(2-oxo-1,2-dihydroquinolin-4-yl)oxy]biphenyl-3-yl}but-2-enamide

A solution of (E)-4-(dimethylamino)-2-butenoic acid (1.63 g, 10 mmol) and excess of 2.0 M oxalyl chloride (3 ml) was refluxed at 60° C. until the solid was in solution. To a separate flask was added 4-[(3′-aminobiphenyl-3-yl)oxy]quinolin-2(1H)-on (329 mg 1.0 mmol) and N,N-dimethylacetamide (2 ml) and placed in an ice bath for ten minutes. The mixture containing the acid chloride was cooled to room temperature and evaporated to dryness. Dichloromethane (2 ml) was added and transferred to the flask in the ice bath slowly. The mixture was allowed to stir in the ice bath for 3 hours. It was evaporated and purified by column chromatography with 10% methanol: 90% dichloromethane: 1.5% ammonium hydroxide to give the desired product (143 mg, 35% yield) as a brown solid.

### Procedure 28:

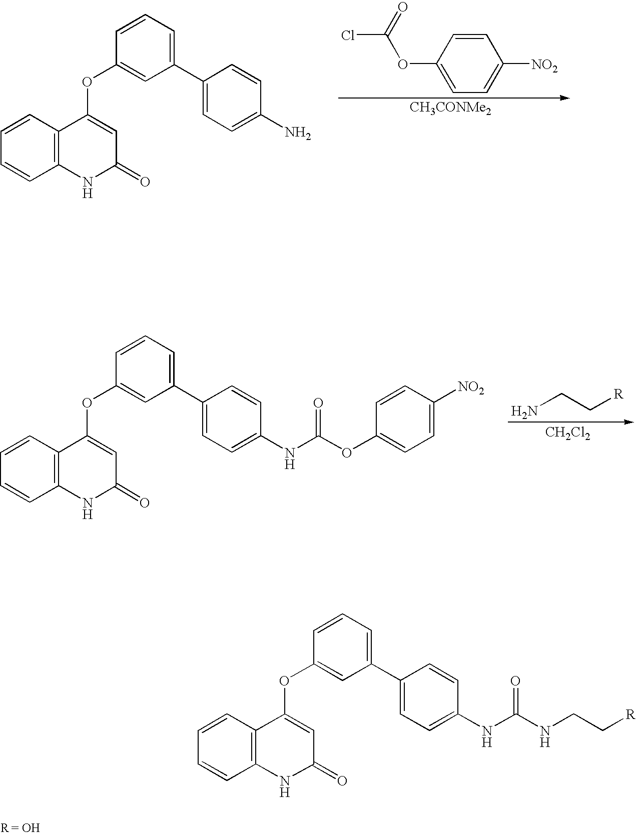

### Example 94

### Synthesis of 1-(2-hydroxyethyl)-3-{3′-[(2-oxo-1,2-dihydroquinolin-4-yl)oxy]biphenyl-4-yl}urea

A solution of 4-[(4′-aminobiphenyl-3-yl)oxy]quinolin-2(1H)-one (310 mg, 0.95 mmol) in N,N-dimethylacetamide (5 ml) was placed in an ice bath for five minutes. To this was added 4-nitrochloroformate (1.91 g, 9.45 mmol) and allowed to stir at room temperature for 2 hours. Water (15 ml) was added to the mixture and the precipitate was filtered, and washed with ethyl ether and acetonitrile to give 400 mg of 4-nitrophenyl 3′-(2-oxo-1,2-dihydroquinolin-4-yloxy)biphenyl-4-ylcarbamate as a yellow solid (86%).

A solution of 4-nitrophenyl 3′-(2-oxo-1,2-dihydroquinolin-4-yloxy) biphenyl-4-ylcarbamate (200 mg, 0.41 mmol) and methylene chloride (15.0 ml) was stirred at room temperature, to this mixture was added ethanolamine excess (3 ml) and the product precipitate out of solution. The mixture was allowed to stir at room temperature for 1 hour, then the solid was filtered and washed with water, ethyl ether and methanol to give the desired product 1-(2-hydroxyethyl)-3-{3′-[(2-oxo-1,2-dihydroquinolin-4-yl)oxy]biphenyl-4-yl}urea as a yellow solid (142 mg, 84%).

The following examples were prepared according to the procedure of Example 94.

| Ex. # | R | Yield (%) |
|---|---|---|
| 94 | OH | 84 |
| 95 | 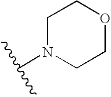 | 56 |
| 96 | NMe₂ | 54 |
| 97 | OMe | 70 |

### Procedure 29:

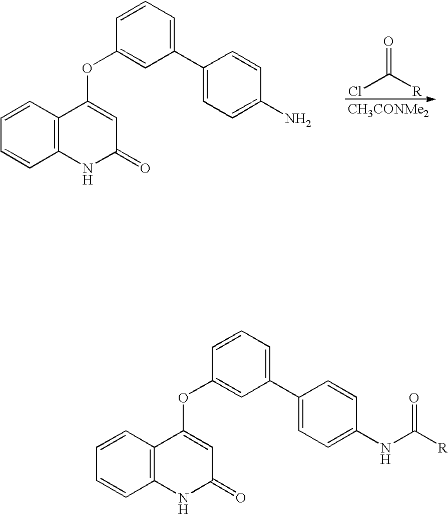

### Example 98

### Synthesis of N-{3′-[(2-oxo-1,2-dihydroquinolin-4-yl)oxy]biphenyl-4-yl}acetamide

A solution of 4-[(4′-aminobiphenyl-3-yl)oxy]quinolin-2(1H)-one (100 mg, 0.3 mmol) in N,N-dimethylacetamide (2 ml) was placed in an ice bath for five minutes. To this was added acetyl chloride (0.22 ml, 3.1 mmol) and allowed to stir at room temperature for 2 hours. Water (5 ml) was added to the mixture and the precipitate was filtered, and washed with ethyl ether and acetonitrile to give 69 mg of synthesis of N-{3′-[(2-oxo-1,2-dihydroquinolin-4-yl)oxy]biphenyl-4-yl}acetamide, as a white solid (61%).

The following examples were prepared according to the procedure of Example 98.

| Ex. # | R | Yield (%) |
|---|---|---|
| 98 | Me | 61 |
| 99 | O-i-Pr | 68 |

### Procedure 30:

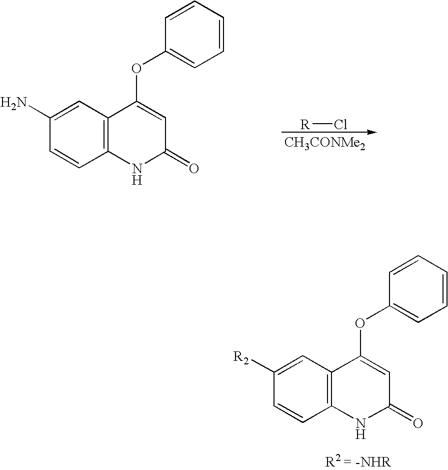

### Example 18

### Synthesis of N-(2-oxo-4-phenoxy-1,2-dihydroquinolin-6-yl)acetamide

A solution of 6-amino-4-phenoxyquinolin-2(1H)-one (50 mg, 0.2 mmol) in N,N-dimethylacetamide (2 ml) was placed in an ice bath for five minutes. To this was added acetyl chloride (0.14 ml, 2.0 mmol) and allowed to stir at room temperature for 30 minutes. Water (3 ml) was added to the mixture and the precipitate was filtered, and washed with ethyl ether and acetonitrile to give 48 mg of N-(2-oxo-4-phenoxy-1,2-dihydroquinolin-6-yl)acetamide, as a white solid (83%).

The following examples were prepared according to the procedure of Example 18.

| Ex. # | R² | Yield (%) |
|---|---|---|
| 18 | 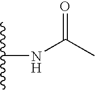 | 83 |
| 22 | 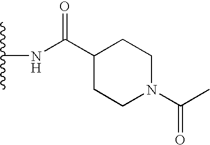 | 50 |
| 23 | 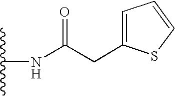 | 54 |
| 24 | 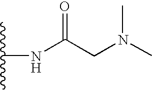 | 67 |

### Synthesis of Intermediates

### Synthesis of substituted 4-hydroxyquinolin-2(1H)-one

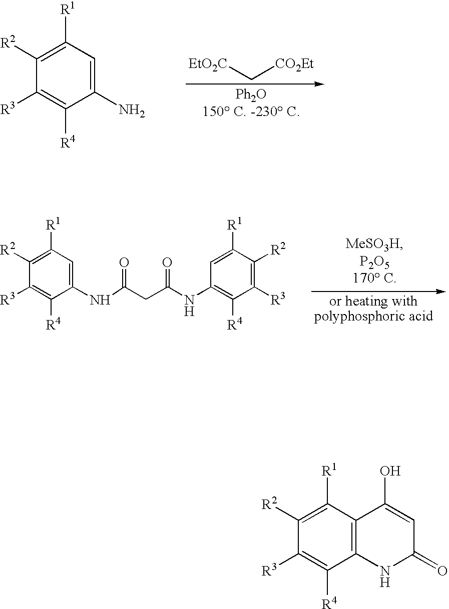

References: Kappe, T., et. al. J. Heterocyclic Chem., 25, 857 (1988) R¹═R⁴═Cl; R²═R⁴═Cl; R³═Cl, R⁴═OMe; and Lee, B. S., et. al. Bioorg. Med. Chem. Lett. 12, 811-815 (2002) R²═NO₂.

| | | | | | ESMS(⁺) |
|---|---|---|---|---|---|
| R¹ | R² | R³ | R⁴ | Yield % | m/z |
| H | Cl | H | H | 57 | 323 |

### Alternate synthesis of substituted 4-hydroxyquinolin-2(1H)-one

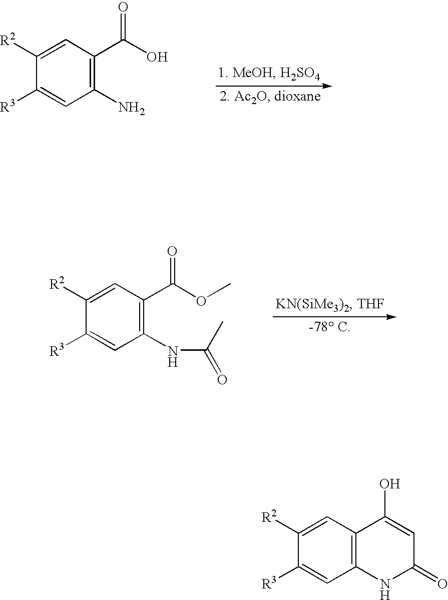

Ref: J. Med. Chem. 47 (24), 5923-36 (2004)

The following compounds were made by this alternate synthesis:

| | | | ESMS(⁺) |
|---|---|---|---|
| R² | R³ | Yield (%) | m/z |
| Cl | H | 87 | 196.3 |
| H | Cl | 85 | 196.0 |
| H | Br | 77 | 241.0 |
| Br | H | 66 | 240.0 |
| NO₂ | H | 71 | 206.15 |

### Synthesis of (diacetoxyiodo)arenes in acetic acid

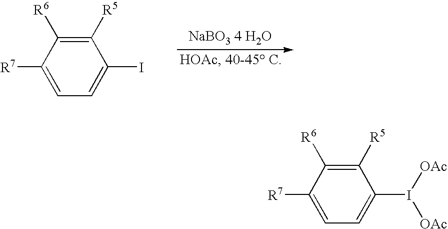

Ref: Tetrahedron 45, 3299-06 (1989), using sodium perborate and acetic acid:

| R⁵ | R⁶ | R⁷ |
|---|---|---|
| H | H | H |
| CH₃ | H | H |
| H | H | CH₃ |
| CH₃ | H | CH₃ |
| H | H | Cl |
| H | H | I |
| H | CF₃ | H |
| H | OCH₃ | H |
| H | H | OCH₃ |
| H | NO₂ | H |

Using this method, the following compounds were synthesized:

| R⁵ | R⁶ | R⁷ | Yield (%) |
|---|---|---|---|
| H | Br | H | 98 |
| H | CF₃ | H | 62 |
| H | H | OCF₃ | 60 |
| H | CH₂NH₂ | H | 16 |
| Br | H | H | 43 |
| H | OCF₃ | H | 11 |
| H | CH₃ | CH₃ | 46 |

### Synthesis of (diacetoxyiodo)arenes in acetic acid and trifluorosulfonic acid

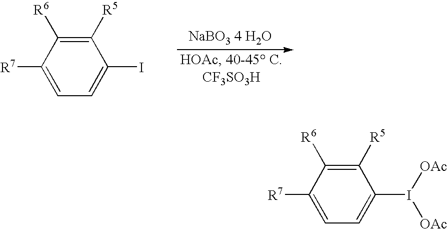

Ref: J. Org. Chem., 70, 6984-6 (2005):

| R⁵ | R⁶ | R⁷ |
|---|---|---|
| H | H | H |
| H | H | CH₃ |
| H | H | Cl |
| H | CF₃ | H |
| H | NO₂ | H |
| H | OCH₃ | H |
| H | H | F |

Using this method, the following compounds were synthesized:

| R⁵ | R⁶ | R⁷ | Yield (%) |
|---|---|---|---|
| H | H | Br | 52 |

### Biological Evaluation—PI3Kα Fluorescence Polarization Assay Protocol

The reaction buffer was 20 mM HEPES pH7.5, 2 mM MgCl₂, 0.05% CHAPS, and 0.01% βME (added fresh). The substrate solution was 40 μM PIP2 (diC8, Echelon, Salt Lake City Utah cat #P-4508, 1 mM in water) and 50 μM ATP in the reaction buffer. Nunc 384-well black polypropylene fluorescent plates were used for PI3K assays. The assay is run by putting 9.5 μl of freshly diluted enzyme in the reaction buffer per well, adding 0.5 μl of diluted drug or DMSO, and mixing. Then 10 μl of the substrate solution is added to each well to start the reaction. A final concentration of 20 μM PIP2 and 25 μM ATP in the reaction was used. Reactions were allowed to proceed for 30-60 minutes at room temperature. After 30-60 minutes, 20 μl of a solution of 10 nM TAMRA detector (Red detector probe-Echelon) and 2.5 uM of GST-murine GRP (1.5 mg/ml in 17% glycerol) was added per well to stop the reaction. The resulting solution was mixed well and allowed to stand for 90-110 minutes before reading plate. Assay Plates were read on Perkin-Elmer Envision plate readers with appropriate filters for Tamra [BODIPY-TMRI(1,3,4,5)P4]. Data obtained were used to calculate enzymatic activity and enzyme inhibition by inhibitor compounds. It is important to keep Red probe solutions dark. This procedure is adapted from Echelon Biosciences Inc procedure for their PI3-Kinase fluorescence polarization activity Assay kit Product number K-1100.

### mTOR Enzyme Assay

The routine human TOR assays with purified enzyme were performed in 96-well plates by DELFIA format as follows. Enzymes were first diluted in kinase assay buffer (10 mM HEPES (pH 7.4), 50 mM NaCl, 50 mM β-glycerophosphate, 10 mM MnCl₂, 0.5 mM DTT, 0.25 μM microcystin LR, and 100 μg/ml BSA). To each well, 12 μL of the diluted enzyme were mixed briefly with 0.5 μL test inhibitor or the control vehicle dimethylsulfoxide (DMSO). The kinase reaction was initiated by adding 12.5 μL kinase assay buffer containing ATP and His6-S6K to give a final reaction volume of 25 μL containing 800 ng/ml FLAG-TOR, 100 μM ATP and 1.25 μM His6-S6K. The reaction plate was incubated for 2 hours (linear at 1-6 hours) at room temperature with gentle shaking and then terminated by adding 25 μL Stop buffer (20 mM HEPES (pH 7.4), 20 mM EDTA, 20 mM EGTA). The DELFIA detection of the phosphorylated (Thr-389) His6-S6K was performed at room temperature using a monoclonal anti-P(T389)-p70S6K antibody (1A5, Cell Signaling) labeled with Europium-N-1-ITC (Eu) (10.4 Eu per antibody, PerkinElmer). The DELFIA Assay buffer and Enhancement solution were purchased from PerkinElmer. A 45 μL portion of the terminated kinase reaction mixture was transferred to a MaxiSorp plate (Nunc) containing 55 μL PBS. The His6-S6K was allowed to attach for 2 hours after which the wells were aspirated and washed once with PBS. 100 μL of DELFIA Assay buffer with 40 ng/ml Eu-P(T389)-S6K antibody was added. The antibody binding was continued for 1 hour with gentle agitation. The wells were then aspirated and washed 4 times with PBS containing 0.05% Tween-20 (PBST). 100 μL of DELFIA Enhancement solution was added to each well and the plates were read in a PerkinElmer Victor model plate reader. Data obtained were used to calculate enzymatic activity and enzyme inhibition by potential inhibitors

### In Vitro Cell Growth Assay

Cell lines used were human adenocarcinoma (LoVo), pancreatic (PC3), prostate (LNCap), breast (MDA468, MCF7), colon (HCT116), renal (HTB44 A498), and ovarian (OVCAR3) tumor cell lines. The tumor cells were plated in 96-well culture plates at approximately 3000 cells per well. One day following plating, various concentrations of inhibitors in DMSO were added to cells (final DMSO concentration in cell assays was 0.25%). Three days after drug treatment, viable cell densities were determined by cell mediated metabolic conversion of the dye MTS, a well-established indicator of cell proliferation in vitro. Cell growth assays were performed using kits purchased from Promega Corporation (Madison, Wis.), following the protocol provided by the vendor. Measuring absorbance at 490 nm generated MTS assay results. Compound effect on cell proliferation was assessed relative to untreated control cell growth. The drug concentration that conferred 50% inhibition of growth was determined as IC₅₀ (INA).

Following the above protocols, the following biological data was obtained:

| | mTOR Kinase | PI3Kα | LNCap | MDA468 |
|---|---|---|---|---|
| Structure | IC₅₀ (μM) | IC₅₀ (nM) | IC₅₀ (μM) | IC₅₀ (μM) |
| 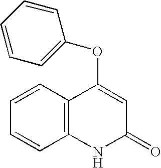 | 0.37 | 24128 | 8.88 | 60 |
| 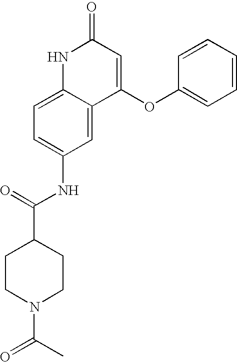 | >20.000 | >10000 | >60.00 | >60.00 |
| 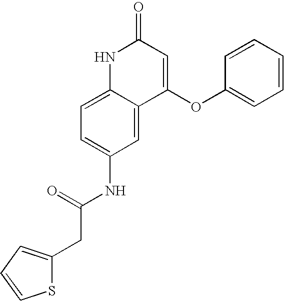 | 14.50 | 383 | 22 | 35 |
| 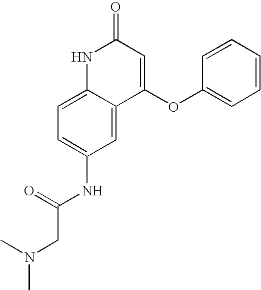 | >20.000 | >10000 | >60.00 | >60.00 |
| 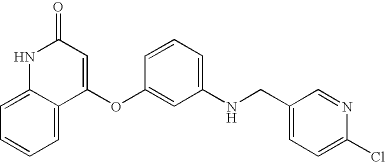 | 0.06 | 13000 | 3.5 | 8 |
| 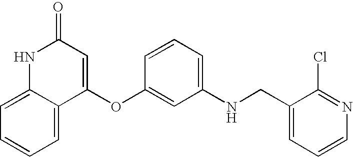 | 0.13 | 11000 | 5.3 | >60.00 |
| 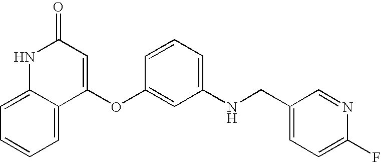 | 0.08 | 9539 | 4 | 11 |
| 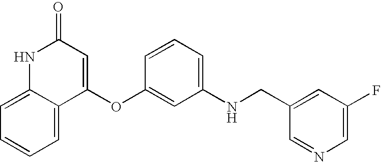 | 0.05 | 8992 | 1.9 | 7.2 |
| 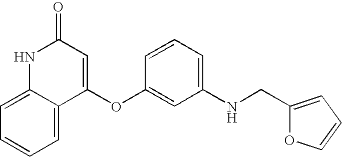 | 0.13 | 4645 | 3.4 | 13 |
| 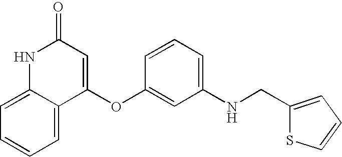 | 0.08 | >10000 | 5.4 | >60.00 |
| 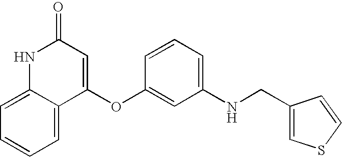 | 0.12 | >10000 | 35 | |
| 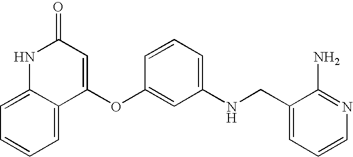 | 0.07 | >10000 | 4 | |
| 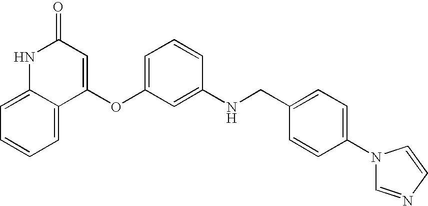 | 0.08 | >10000 | 4 | |
| 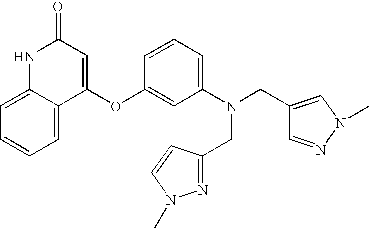 | 0.23 | 4619 | 3 | 3 |
| 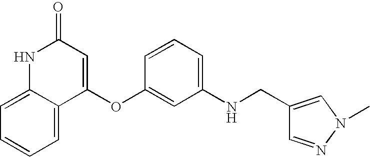 | 0.17 | 4794 | 4 | 8.8 |
| 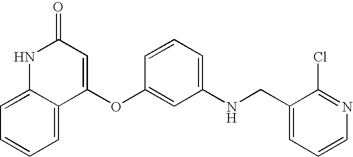 | 0.15 | >10000 | 4.2 | 10 |
| 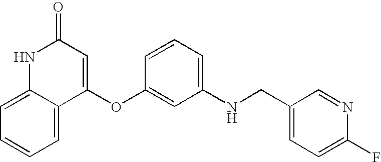 | 0.24 | | 13 | 50 |
| 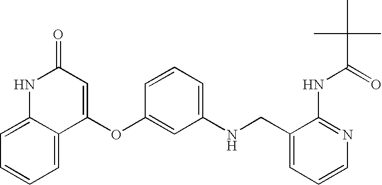 | 0.04 | 4053 | 1.3 | 2.8 |
| 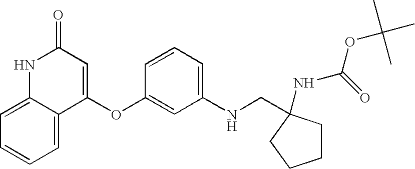 | 0.23 | 5130 | 4.1 | 8.5 |
| 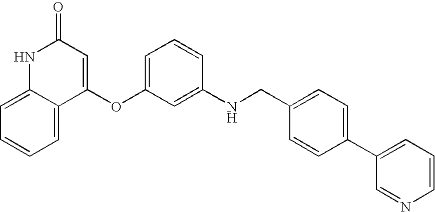 | 0.09 | 7976 | 3.3 | 8 |
| 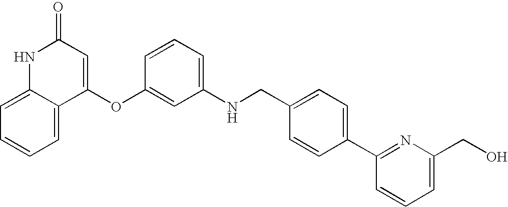 | 0.09 | 3919 | 4.3 | 10 |
| 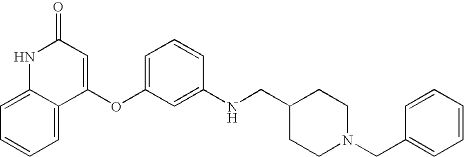 | 0.18 | 9535 | 3 | 4.1 |
| 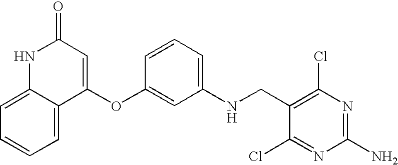 | 0.17 | >10000 | 5.2 | 3.5 |
| 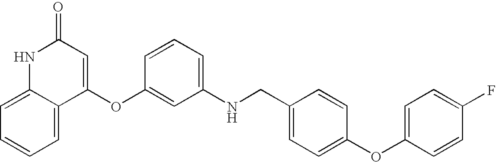 | 3.50 | >10000 | 5.3 | 12 |
| 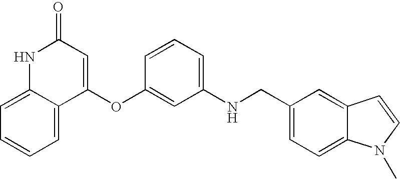 | 0.35 | 1425 | 1 | 1.2 |
| 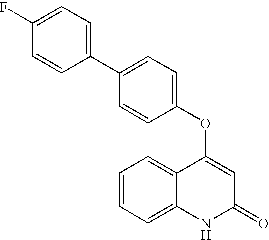 | 15.75 | 9655 | >60.00 | >60.00 |
| 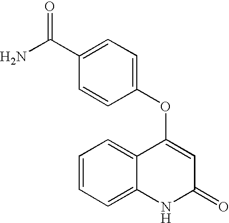 | 1.83 | >10000 | >60.00 | 23 |
| 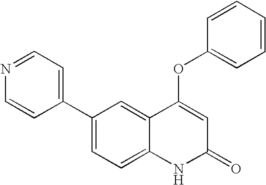 | 20.00 | 3941 | 60 | 60 |
| 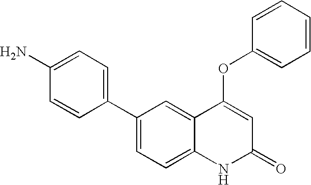 | >20.000 | 6535 | 18 | 60 |
| 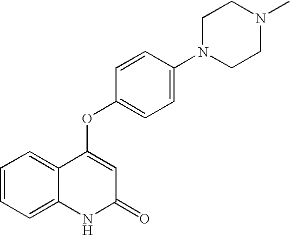 | 7.00 | >10000 | 17 | 40 |
| 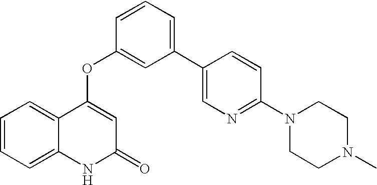 | 0.21 | >10000 | 4 | 9 |
| 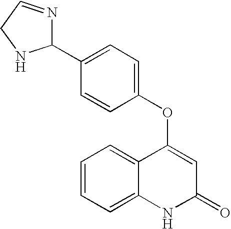 | 0.78 | >10000 | 45 | 60 |
| 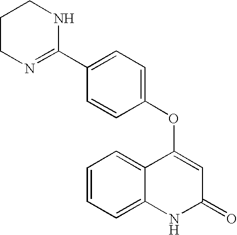 | 0.57 | >10000 | >60.00 | 60 |
| 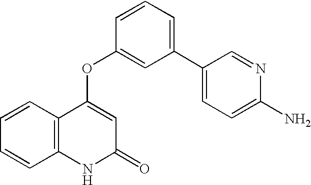 | 0.09 | 9000 | 23 | 1.95 |
| 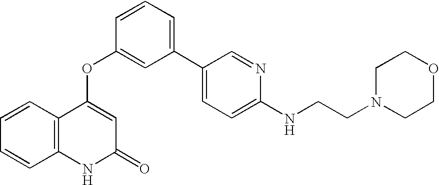 | 0.15 | 6786 | 4.7 | 2 |
| 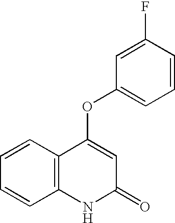 | 1.12 | >10000 | >60.00 | >60.00 |
| 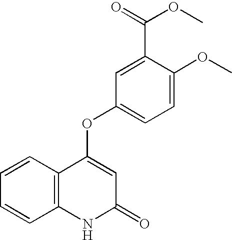 | 1.28 | >10000 | 7.9 | >60.00 |
| 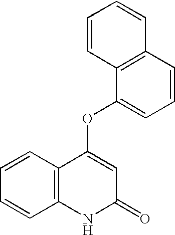 | 0.23 | >10000 | >60.00 | >60.00 |
| 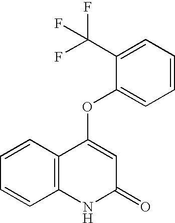 | 1.65 | 308 | >60.00 | >60.00 |
| 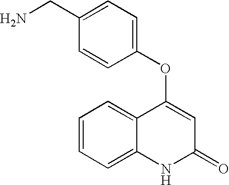 | 3.95 | >10000 | 12 | 35 |
| 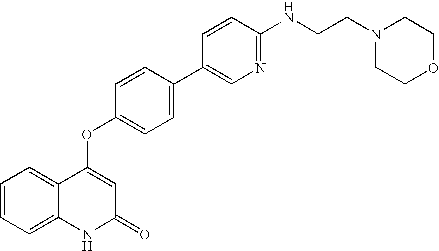 | 1.08 | >10000 | 1.05 | 8 |
| 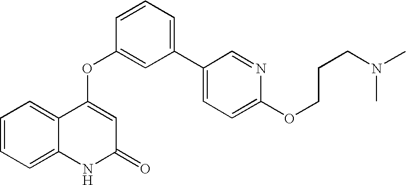 | 0.08 | >10000 | 3.8 | 10 |
| 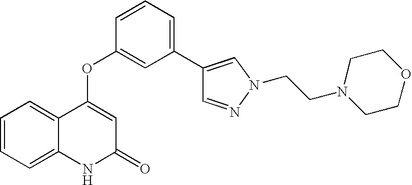 | 0.14 | >10000 | 2.2 | 10 |
| 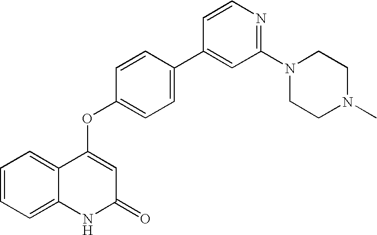 | 0.56 | 9000 | 0.8 | 1.2 |
| 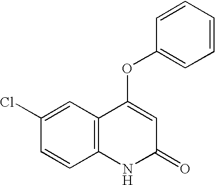 | 6.75 | 5143 | 13 | >60.00 |
| 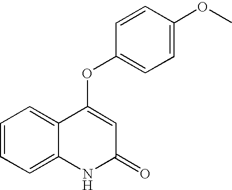 | 0.54 | 10984 | >60.00 | >60.00 |
| 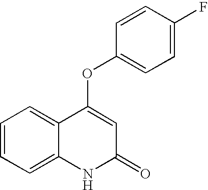 | 0.60 | 1585 | 18 | >60.00 |
| 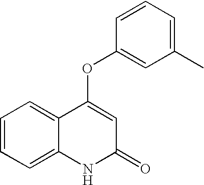 | 0.24 | 736 | 7.5 | 20 |
| 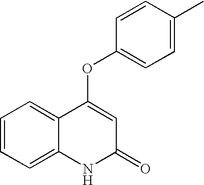 | 1.15 | 4171 | 18 | >60.00 |
| 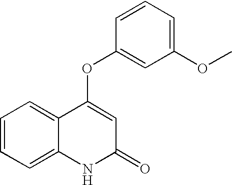 | 0.30 | >10000 | 9.3 | 60 |
| 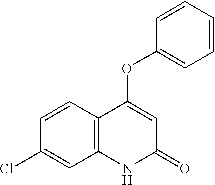 | >20.000 | 308 | | |
| 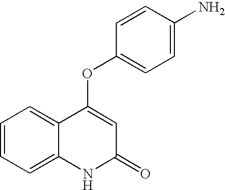 | 0.16 | 2225 | 7.5 | 27 |
| 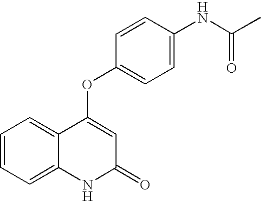 | 6.00 | 7284 | | |
| 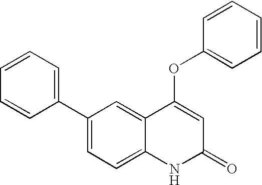 | 8.20 | 8230 | | |
| 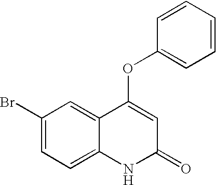 | >20.000 | >10000 | | |
| 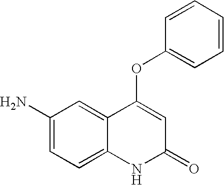 | >20.000 | 2572 | >60.00 | >60.00 |
| 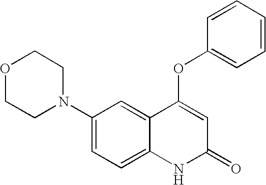 | 16.50 | 1174 | 7.5 | 27 |
| 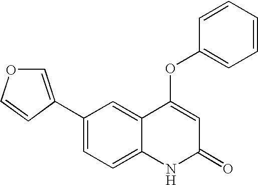 | 7.30 | 6955 | >60.00 | 4.5 |
| 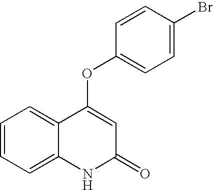 | 2.20 | 4111 | >60.00 | 50 |
| 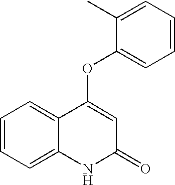 | 2.00 | 7907 | 8 | >60.00 |
| 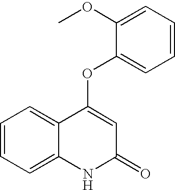 | 9.10 | 4683 | >60.00 | >60.00 |
| 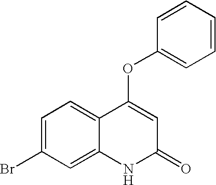 | >20.000 | 5387 | >60.00 | >60.00 |
| 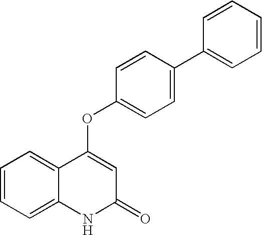 | >20.000 | 938 | >60.00 | >60.00 |
| 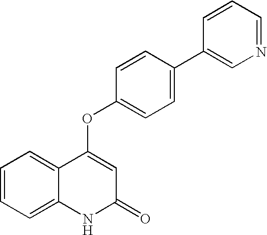 | 1.75 | 3286 | 2.15 | 16.65 |
| 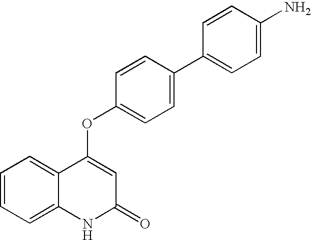 | >20.000 | 778 | 2 | 60 |
| 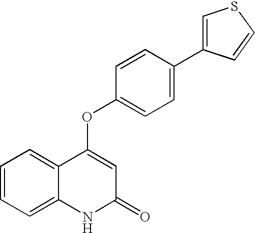 | >20.000 | 2506 | >60.00 | >60.00 |
| 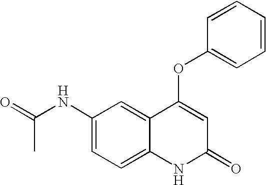 | >20.000 | >10000 | >60.00 | >60.00 |
| 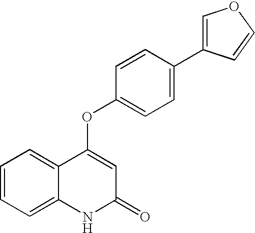 | >20.000 | 1936 | >60.00 | >60.00 |
| 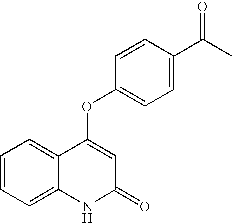 | 1.85 | 1658 | 13 | >60.00 |
| 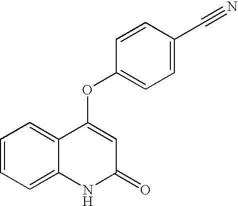 | 7.60 | 2088 | 5.5 | 48 |
| 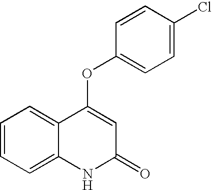 | 0.96 | 2484 | >60.00 | 49 |
| 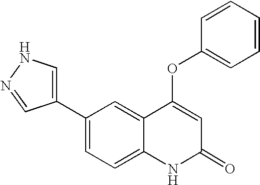 | 19.00 | 375 | >60.00 | >60.00 |
| 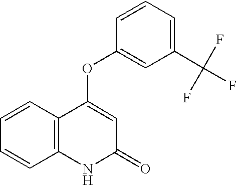 | 1.35 | 8694 | >60.00 | >60.00 |
| 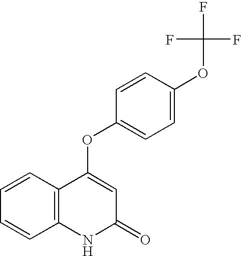 | >20.000 | >10000 | >60.00 | >60.00 |
| 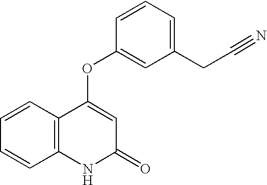 | 0.30 | 5799 | 4.2 | 22 |
| 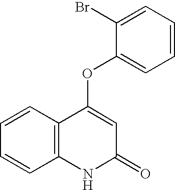 | 0.95 | 8000 | 7 | 40 |
| 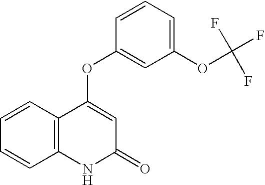 | 0.75 | 8000 | 7 | 60 |
| 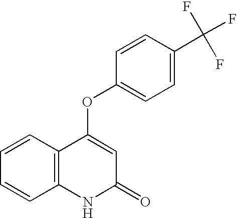 | >20.000 | 2311 | >60.00 | >60.00 |
| 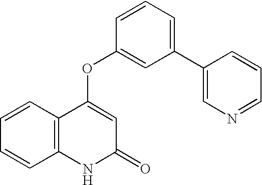 | 0.43 | 3662 | 7 | 20 |
| 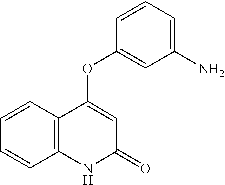 | 0.15 | 106 | 2.5 | 28 |
| 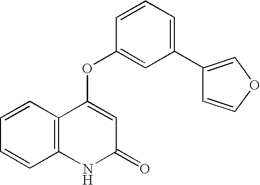 | 0.35 | 4320 | 6.2 | 0.3 |
| 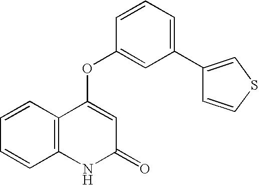 | 0.63 | 630 | 30 | 60 |
| 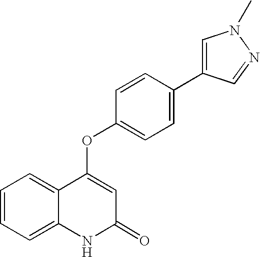 | 1.98 | 2284 | 1.3 | >60.00 |
| 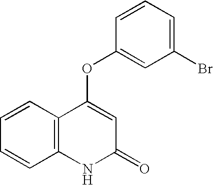 | 0.11 | >10000 | 60 | >60.00 |
| 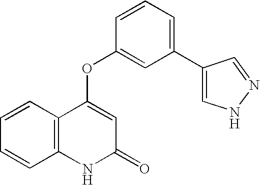 | 0.30 | 3896 | 8 | 2.9 |
| 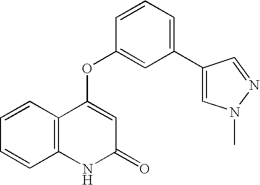 | 0.18 | >10000 | >60.00 | 60 |
| 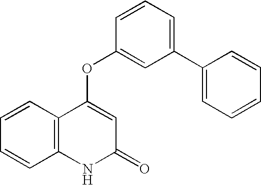 | 0.63 | >10000 | 17 | 60 |
| 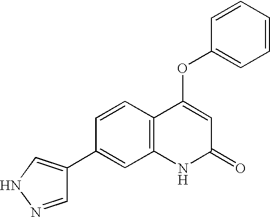 | >20.000 | 2763 | >60.00 | >60.00 |
| 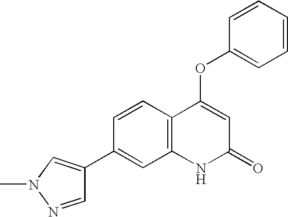 | >20.000 | 4629 | >60.00 | >60.00 |
| 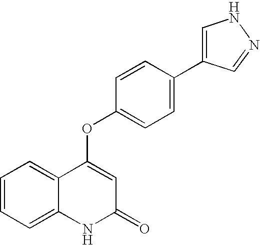 | >20.000 | 2171 | 60 | 60 |
| 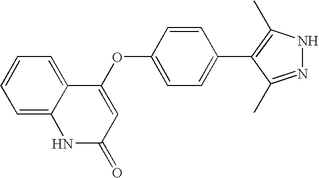 | 1.85 | 6947 | >60.00 | >60.00 |
| 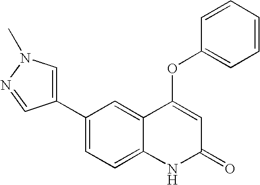 | >20.000 | >10000 | >60.00 | >60.00 |
| 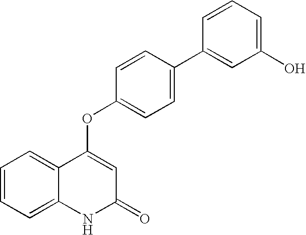 | 0.71 | 1660 | >60.00 | >60.00 |
| 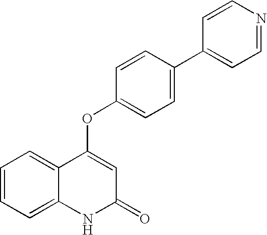 | 0.24 | 4882 | 1.9 | >60.00 |
| 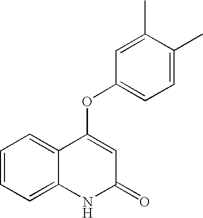 | 0.69 | >10000 | >60.00 | >60.00 |
| 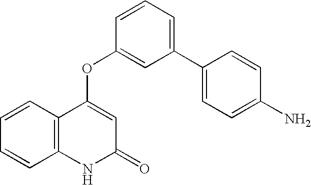 | 0.29 | 9522 | 11 | 30 |
| 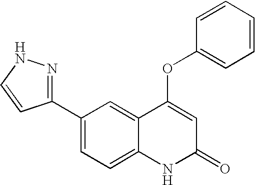 | 11.50 | 1018 | >60.00 | 40 |
| 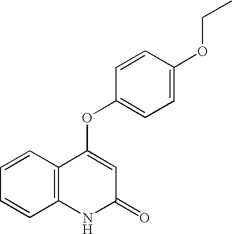 | >20.000 | >10000 | >60.00 | >60.00 |
| 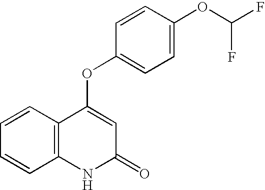 | 1.04 | >10000 | 5.5 | >60.00 |
| 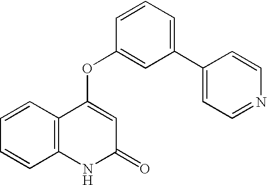 | 0.37 | >10000 | 7 | 60 |
| 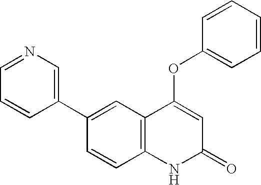 | 8.50 | 7436 | >60.00 | >60.00 |
| 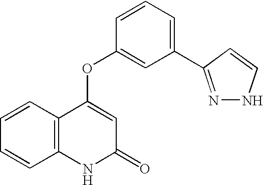 | 0.15 | 6668 | 30 | 60 |
| 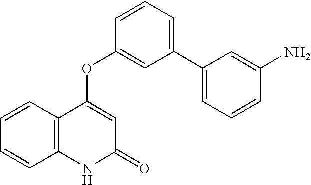 | 0.17 | 5591 | 8 | 30 |
| 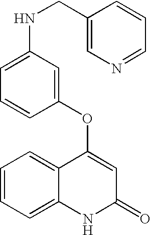 | 0.08 | >10000 | 3.2 | 9.77 |
| 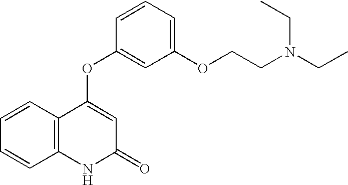 | 0.32 | >10000 | 7 | 28 |
| 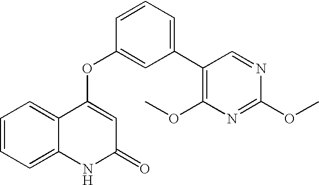 | 0.28 | >10000 | >60.00 | 38 |
| 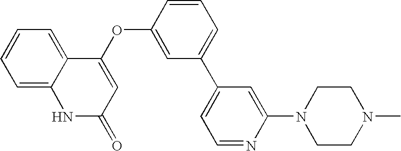 | 0.40 | >10000 | 2.5 | 4.05 |
| 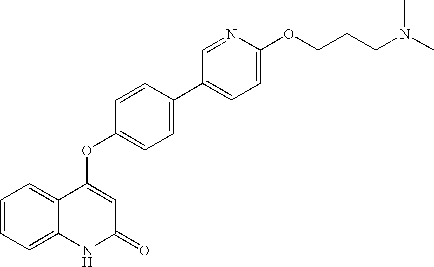 | 0.82 | >10000 | 2.1 | 3.9 |
| 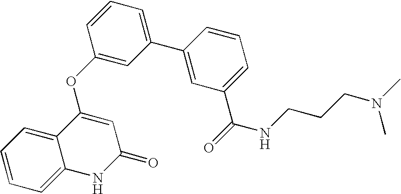 | 0.50 | >10000 | 4.3 | 10 |
| 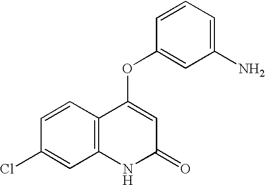 | 0.60 | 6731 | 4.5 | >60.00 |
| 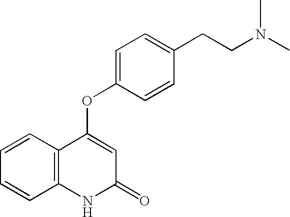 | 3.23 | >10000 | 8 | 23 |
| 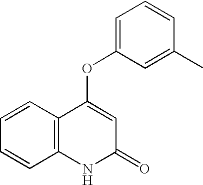 | 0.50 | >10000 | 60 | 40 |
| 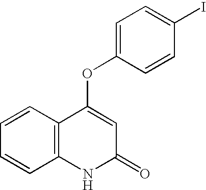 | 0.74 | >10000 | >60.00 | 18 |
| 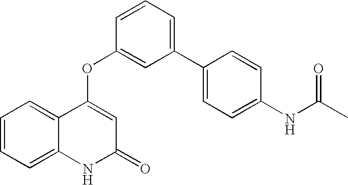 | 0.22 | 3672 | 15 | 11 |
| 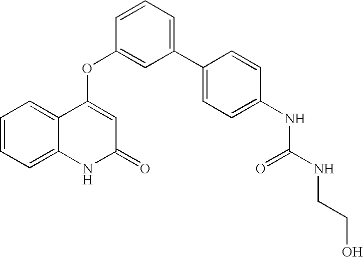 | 0.25 | 8485 | 60 | >60.00 |
| 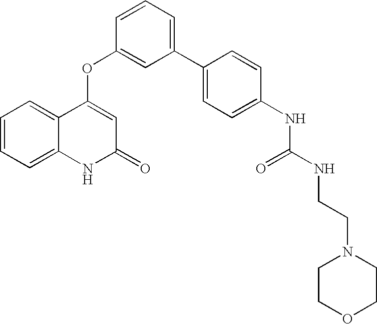 | 0.22 | >10000 | 7 | 25 |
| 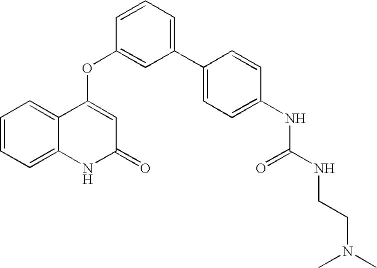 | 0.10 | >10000 | 7 | 9 |
| 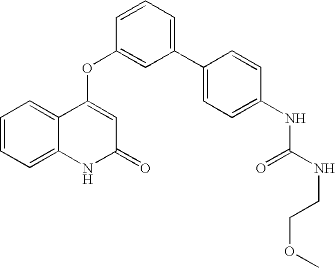 | 0.26 | >10000 | >60.00 | 60 |
| 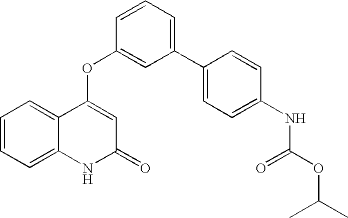 | 1.35 | >10000 | 4 | 5 |
| 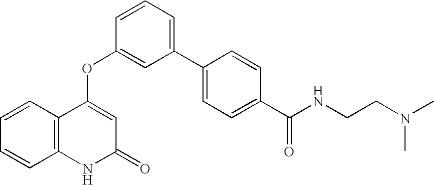 | 0.24 | >10000 | 6 | 12 |
| 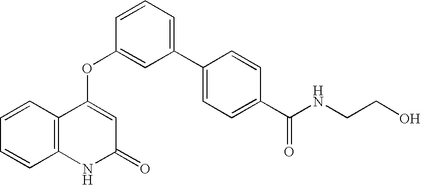 | 0.23 | >10000 | >60.00 | >60.00 |
| 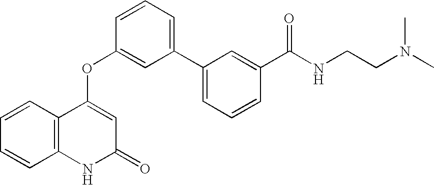 | 0.32 | >10000 | 10 | 10 |
| 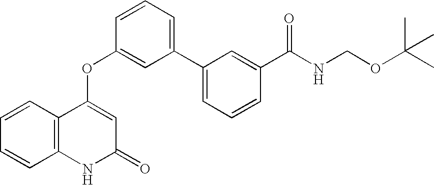 | 2.38 | >10000 | 6.6 | >60.00 |
| 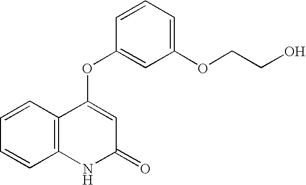 | 0.34 | 12000 | 6 | >60.00 |
| 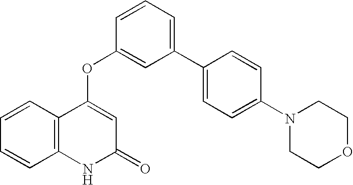 | 0.49 | 10378 | 13 | 35 |
| 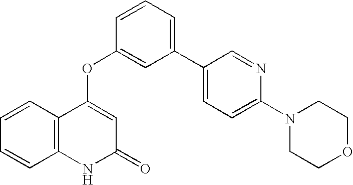 | 0.20 | >10000 | 12 | >60.00 |
| 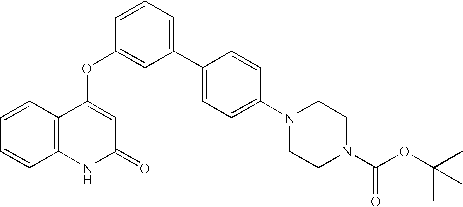 | 5.85 | 9890 | 60 | 44 |
| 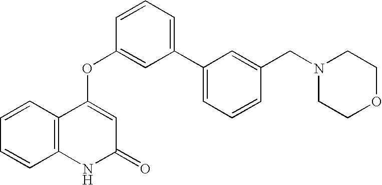 | 0.60 | 6832 | 9.5 | 30 |
| 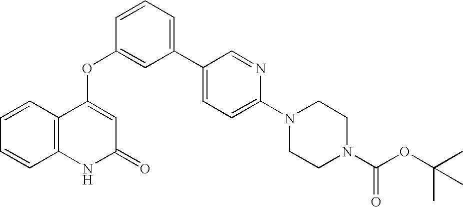 | 3.70 | >10000 | >60.00 | 18 |
| 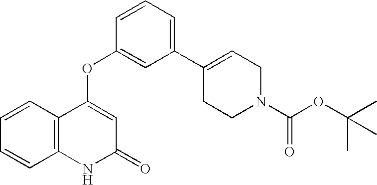 | 0.65 | 9500 | 13 | 32 |
| 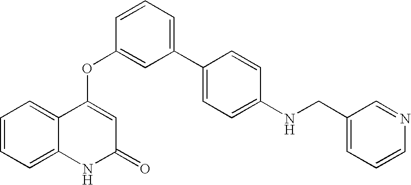 | 1.30 | 7000 | >60.00 | >60.00 |
| 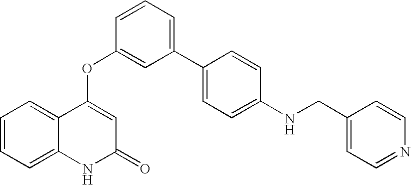 | 0.70 | 5254 | >60.00 | >60.00 |
| 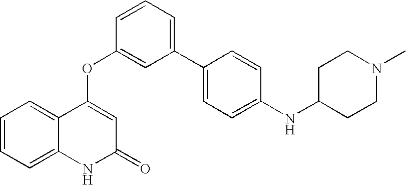 | 0.05 | 8000 | 2.8 | 5 |
| 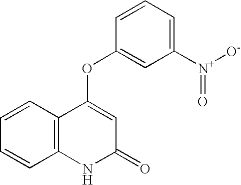 | 2.25 | >10000 | >60.00 | >60.00 |
| 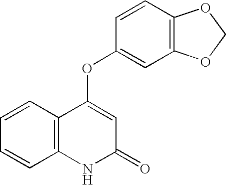 | 1.50 | >10000 | >60.00 | >60.00 |
| 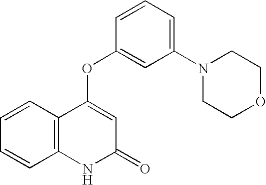 | 0.43 | >10000 | 9 | 60 |
| 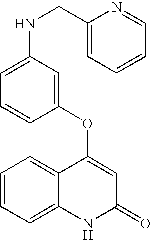 | 0.15 | 3487 | 5.8 | 12 |
| 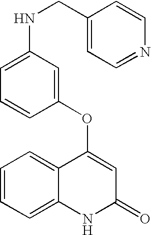 | 0.15 | >10000 | 5 | 10 |
| 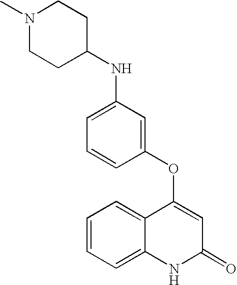 | 0.32 | >10000 | 6 | 12 |
| 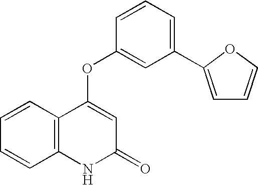 | 0.40 | >10000 | >60.00 | 0.7 |
| 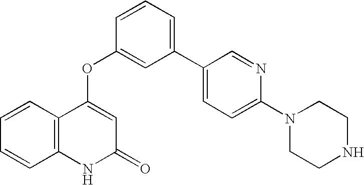 | 0.15 | >10000 | 4.8 | 7 |
| 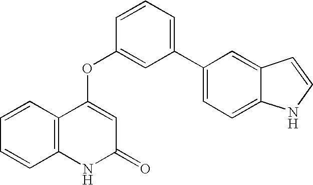 | 0.47 | >10000 | 12 | 23 |
| 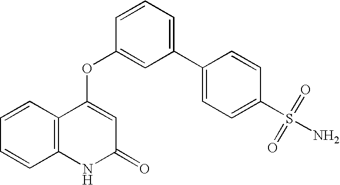 | 0.26 | >10000 | >60.00 | 60 |
| 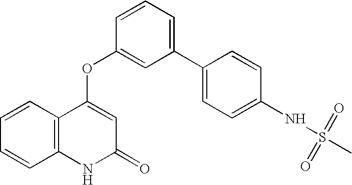 | 0.27 | >10000 | 5.8 | 26 |
| 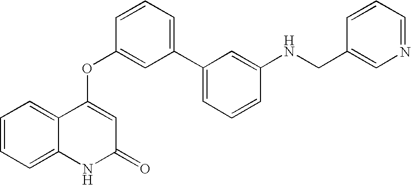 | 0.51 | >10000 | 8 | 28 |
| 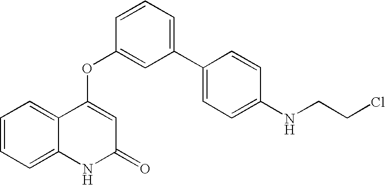 | 0.44 | >10000 | 6.2 | 11 |
| 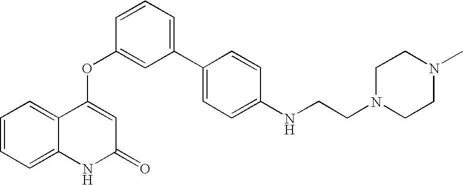 | 0.14 | >10000 | 12 | 23 |
| 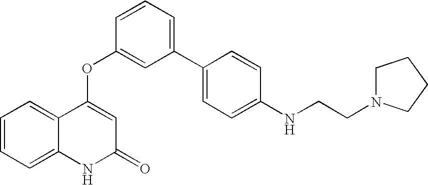 | 0.18 | >10000 | 3.05 | 4.5 |
| 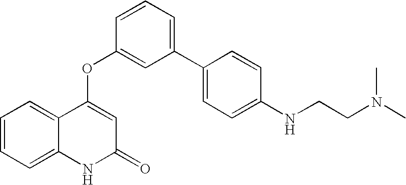 | 0.18 | 5000 | 4.8 | 8 |
| 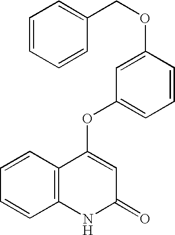 | 20.00 | 4150 | >60.00 | >60.00 |
| 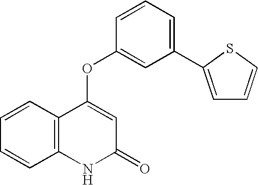 | >20.000 | 2694 | >60.00 | 3.8 |
| 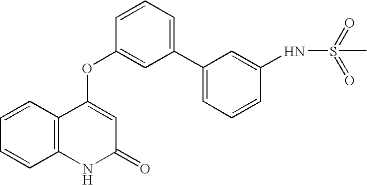 | 0.58 | 3554 | 6.5 | 18 |
| 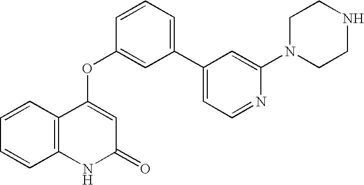 | 0.55 | 15000 | 9 | 8.5 |
| 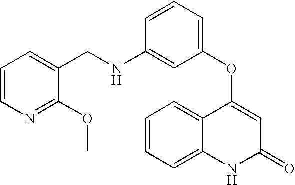 | 0.40 | 6741 | 8.5 | 60 |
| 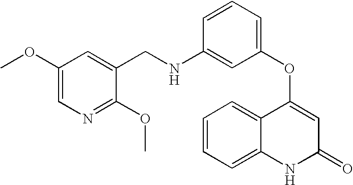 | 0.51 | 1935 | 9 | 15 |
| 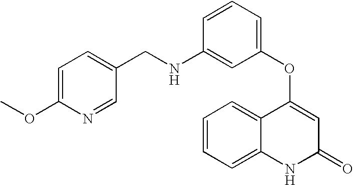 | 0.31 | 4074 | 4.1 | 12 |
| 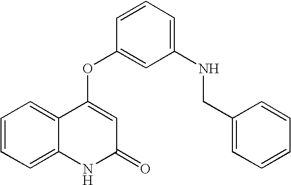 | 0.14 | 3784 | 6 | |
| 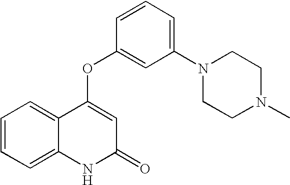 | 0.37 | 10339 | 0.62 | |
| 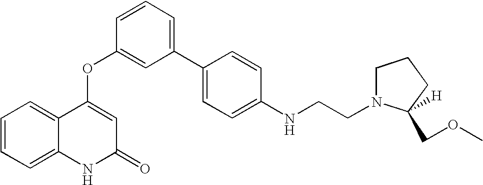 | 0.13 | 11000 | 5 | |
| 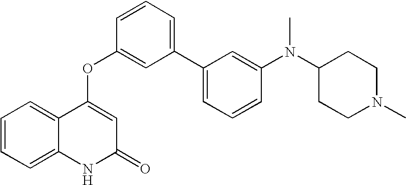 | 0.33 | 8313 | 3 | |
| 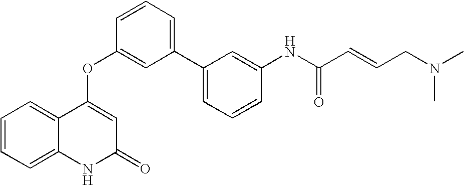 | 0.14 | 12000 | 4 | 8 |
| 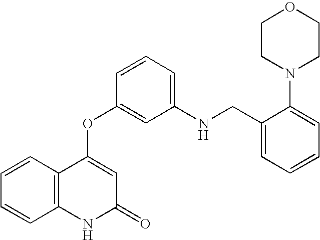 | 0.33 | 5620 | 9 | 24 |
| 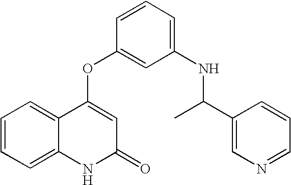 | 0.19 | 3576 | 3.3 | 11 |
| 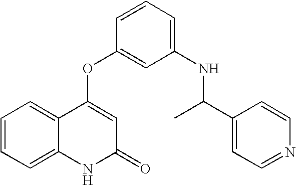 | 0.14 | 7523 | 3.1 | 11 |
| 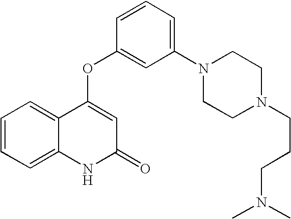 | 0.72 | 10000 | 7 | 10.5 |
| 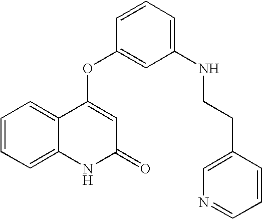 | 0.12 | >10000 | 4 | 11.5 |
| 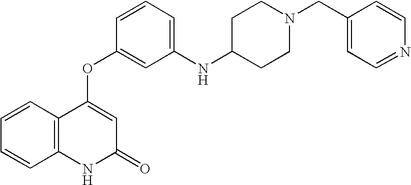 | 0.15 | >10000 | 6.4 | 31 |
| 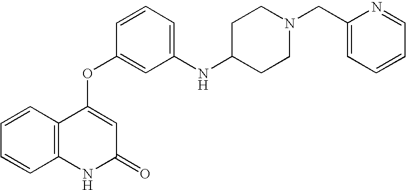 | 0.19 | >10000 | 6.5 | 19 |
| 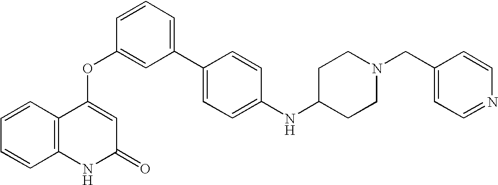 | 0.12 | >10000 | 6.7 | 13 |
| 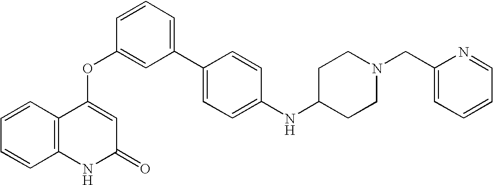 | 0.11 | >10000 | 5.2 | 11 |
| 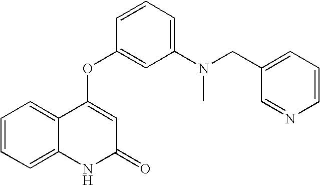 | 0.42 | 1963 | 13 | 20 |
| 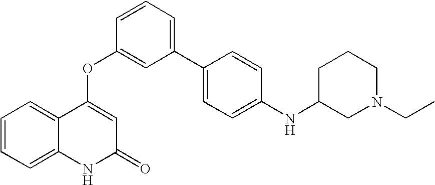 | 0.31 | 7998 | 3.9 | 10 |
| 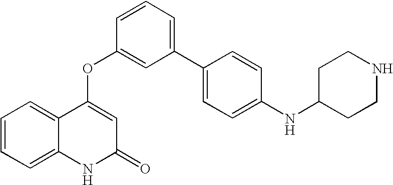 | 0.09 | >10000 | 2.3 | 3.8 |
| 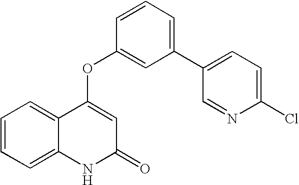 | >20.000 | >10000 | >60.00 | >60.00 |
| 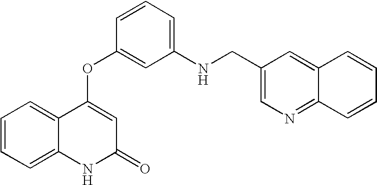 | 0.06 | >10000 | >60.00 | 60 |
| 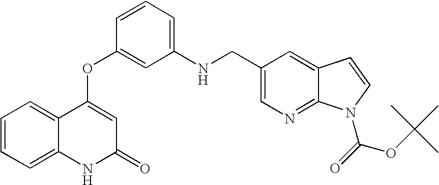 | 4.50 | 11834 | 11.5 | 60 |
| 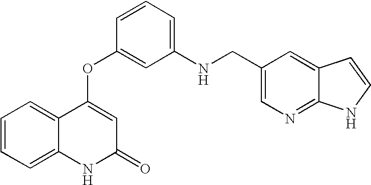 | 0.12 | 3418 | 4.9 | 13 |
| 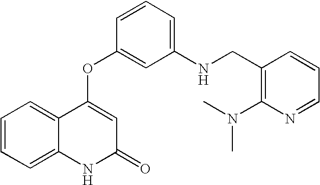 | 0.31 | >10000 | 5 | 12 |
| 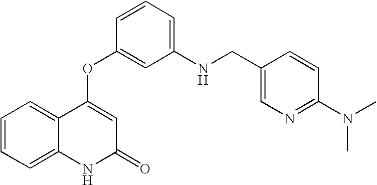 | 0.16 | 4553 | >60.00 | >60.00 |
| 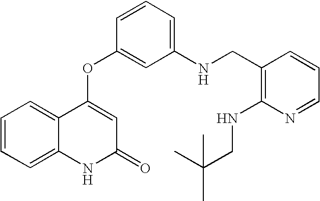 | 0.16 | 10000 | 4.2 | 10 |
| 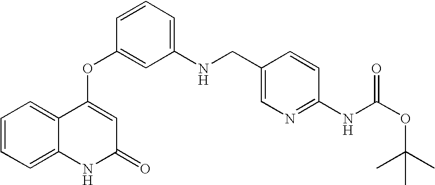 | 0.42 | 10000 | 12 | 60 |
| 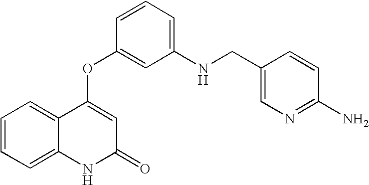 | 0.11 | 7155 | 4 | 8 |
| 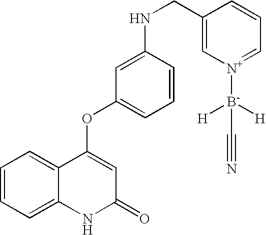 | 0.03 | 7379 | 2.3 | 4.2 |
| 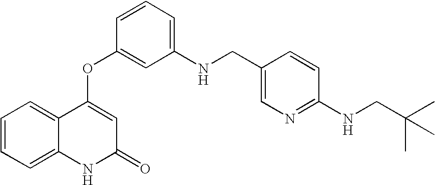 | 0.35 | 2091 | 7 | 12 |
| 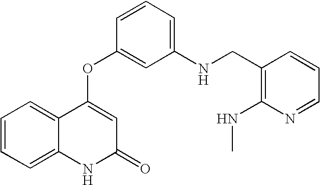 | 0.09 | >10000 | 2.8 | 6.8 |
| 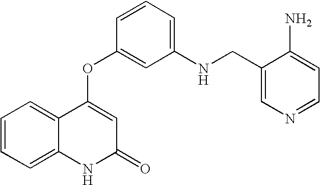 | 0.15 | >10000 | 8 | 11.5 |
| 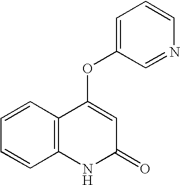 | 1.16 | >10000 | >60.00 | >60.00 |
| 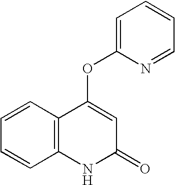 | 9.70 | 8736 | >60.00 | |

Throughout this application, various publications are referenced. The disclosures of these publications in their entireties are hereby incorporated by reference into this application in order to more fully describe the state of the art as known to those skilled therein as of the date of the invention described and claimed herein.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compound of Formula I: 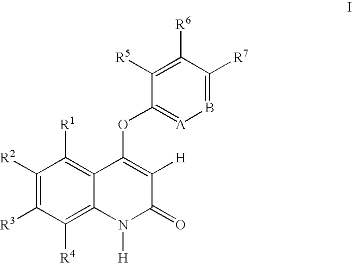
wherein:
A and B are each independently CH or N;
R¹, R², R³, and R⁴ are each independently selected from:
H, halogen, C₆-C₁₀aryl, C₁-C₉heteroaryl, and C₁-C₉heterocyclyl-, C₁-C₆alkyl, C₃-C₆cycloalkyl, C₂-C₆alkenyl, C₂-C₆alkynyl, NHC(O)Y, NR′R″, CF₃, OCF₃, OCHF₂, OR′, SR′, SOR′, SO₂R′, NO₂, SO₂NR′R″, NHSO₂R′, CONR′R″, CN, COOR′, OCOR′, COR′, NHCONR′R″, and NHCOOR';
Y is C₁-C₉heteroaryl-, C₁-C₆alkyl-, or C₁-C₉heterocyclyl- and is optionally substituted with 1 or 2 substituents selected from C₆-C₁₀aryl-, C₁-C₉heteroaryl-, C₁-C₉heterocyclyl-, OR′, SR′, COR′, and NR′R″;
R′ and R″ are each independently selected from the group consisting of H, C₁-C₆alkyl, C₆-C₁₀aryl-, C₁-C₉heteroaryl-, and C₁-C₉heterocyclyl-;
R⁵ and R⁷ are each independently selected from the group consisting of H, HO—, C₁-C₆alkoxy, C₆-C₁₀aryl-O—, C₁-C₉heteroaryl-O—, C₁-C₉heterocyclyl-O—, NHC(O)Y, C₆-C₁₀aryl, C₁-C₉heteroaryl, and C₁-C₉heterocyclyl-, COR′, OCF₃, C₁-C₆alkyl, C₃-C₆cycloalkyl, C₂-C₆alkenyl, C₁-C₆alkynyl, OCHF₂, halogen, COOR′, NO₂, NR′R″, OH, CF₃, and CN;
R⁶ is selected from the group consisting of H, HO—, C₁-C₆alkoxy, O₆—C₁₀aryl-O—, O₁—C₉heteroaryl-O—, C₁-C₉heterocyclyl-O—, NHC(O)Y, O₆—C₁₀aryl, C₁-C₉heteroaryl, C₁-C₉heterocyclyl-, COR′, OCF₃, C₁-C₆alkyl, C₃-C₆cycloalkyl, C₂-C₆alkenyl, C₂-C₆alkynyl, OCHF₂, halogen, COOR′, NO₂, NR′R″, NHCHR′R″, CF₃, CN, and CH₂CN;
or R⁵ and R⁶ taken together with the carbon atoms to which they are attached form a C₆-C₁₀aryl;
or, R⁶ and R⁷ taken together are OCH₂O, so that together with the carbon atoms to which they are attached they form a 5-membered ring;
each C₁-C₆alkyl group may be optionally substituted by a OH, NR′R″, C₃-C₆cycloalkyl, C₆-C₁₀aryl, C₁-C₉heteroaryl, or C₁-C₉heterocyclyl-group;
each C₁-C₉heteroaryl- and C₁-C₉heterocyclyl-group includes 1-4 ring atoms selected from O, S, and N;
and each C₆-C₁₀aryl, C₁-C₉heteroaryl, and C₁-C₉heterocyclyl-group is optionally substituted with one to three substituents independently selected from halogen, C₆-C₁₀aryl, C₁-C₉heteroaryl, C₁-C₉heterocyclyl-, C₁-C₆alkyl, C₃-C₆cycloalkyl, C₂-C₆alkenyl, C₂-C₆alkynyl, NHC(O)Y, NR′R″, CF₃, OCF₃, OCHF₂, OR′, SR′, SOR′, SO₂R′, NO₂, SO₂NR′R″, NHSO₂R′, CONR′R″, CN, COOR′, OCOR′, COR′, NHCONR′R″, and NHCOOR';
provided that: when A and B are both CH, and R⁵, R⁶ and R⁷ are all H, then R¹, R², R³ and R⁴ are not H, halogen, phenyl, naphthyl, C₁-C₆alkyl, C₃-C₆cycloalkyl, C₂-C₆alkenyl, NHC(O)Y, NR′R″, OR′, SR′, NO₂, SO₂NR′R″, NHSO₂R′, CONR′R″, CN, COOR′, OCOR′, COR′, or NHCONR′R″;
further provided that: when A and B are both CH, and R¹, R², R³, R⁴, R⁵, and R⁶ are all H, then R⁷ cannot be H, unsubstituted methyl or unsubstituted methoxy;
and further provided that: when B is N, then R¹, R², R³ and R⁴ are not NO₂, CN, COOR′, CONR′R″, C₃-C₆cycloalkyl-, or C₁-C₉heterocyclyl-,
and pharmaceutically acceptable salts thereof.

2. The compound of claim 1 wherein R¹ is H.

3. The compound of claim 1 wherein R² is H.

4. The compound of claim 1 wherein R³ is H.

5. The compound of claim 1 wherein R⁴ is H.

6. The compound of claim 2 wherein R² or R³ is selected from the group consisting of halogen, C₆-C₁₀aryl, C₁-C₉heteroaryl, C₁-C₉heterocyclyl-, and NHC(O)Y.

7. The compound of claim 1 wherein A and B are both CH.

8. The compound of claim 1 wherein A is CH and B is N.

9. The compound of claim 1 wherein A is N and B is CH.

10. The compound of claim 1 wherein R⁵ is halogen, C₁-C₆alkoxy-, or CF₃, or R⁵ and R⁶ taken together with the carbon atoms to which they are attached form a phenyl ring.

11. The compound of claim 1 wherein R⁶ is selected from the group consisting of C₆-C₁₀aryl, C₁-C₉heteroaryl, C₁-C₉heterocyclyl-, and NHCHR′R″.

12. A compound of claim 1 wherein R¹, R², R³, R⁴, R⁵, and R⁷ are all H.

13. A compound of claim 12 wherein A and B are both CH and R⁶ is selected from the group consisting of C₆-C₁₀aryl, C₁-C₉heteroaryl, C₁-C₉heterocyclyl-, and NHCHR′R″.

14. A compound of claim 12 wherein one of A and B is N and the other is CH.

15. A compound of claim 14 wherein R⁶ is H.

16. A compound selected from the group consisting of:
4-[3-(6-aminopyridin-3-yl)phenoxy]quinolin-2(1H)-one;
4-({4′-[(1-methylpiperidin-4-yl)amino]biphenyl-3-yl}oxy)quinolin-2(1H)-one;
4-{3-[1-(2-morpholin-4-ylethyl)-1H-pyrazol-4-yl]phenoxy}quinolin-2(1H)-one;
4-{3-[pyridin-3-ylmethyl)amino]phenoxy}quinolin-2(1H)-one;
4-(pyridin-2-yloxy) quinolin-2(1H)-one;
4-(pyridin-3-yloxy) quinolin-2(1H)-one;
2,2-dimethyl-N-{3-[({3-[(2-oxo-1,2-dihydroquinolin-4-yl)oxy]phenyl}amino)methyl]-pyridin-2-yl}propanamide;
4-(3-{[(5-fluoropyridin-3-yl)methyl]amino}phenoxy)quinolin-2(1H)-one;
4-({4′-[(1-methylpiperidin-4-yl)amino]biphenyl-3-yl}oxy)quinolin-2(1H)-one;
4-(3-{[(6-chloropyridin-3-yl)methyl]amino}phenoxy)quinolin-2(1H)-one;
4-{3-[(quinolin-3-ylmethyl)amino]phenoxy}quinolin-2(1H)-one;
4-(3-{[(2-aminopyridin-3-yl)methyl]amino}phenoxy)quinolin-2(1H)-one;
4-(3-{6-[3-(dimethylamino)propoxy]pyridin-3-yl}phenoxy)quinolin-2(1H)-one;
4-(3-{[4-(1H-imidazol-1-yl)benzyl]amino}phenoxy)quinolin-2(1H)-one;
4-{3-[(pyridin-3-ylmethyl)amino]phenoxy}quinolin-2(1H)-one;
4-(3-{[(6-fluoropyridin-3-yl)methyl]amino}phenoxy)quinolin-2(1H)-one;
4-{3-[(2-thienylmethyl)amino]phenoxy}quinolin-2(1H)-one;
4-[3-({4-[6-(hydroxymethyl)pyridin-2-yl]benzyl}amino)phenoxy]quinolin-2(1H)-one;
4-{[4′-(piperidin-4-ylamino)biphenyl-3-yl]oxy}quinolin-2(1H)-one;
4-[3-{[2-(methylamino)pyridin-3-yl]methyl}amino)phenoxy]quinolin-2(1H)-one;
4-[3-(6-aminopyridin-3-yl)phenoxy]quinolin-2(1H)-one;
4-{3-[(4-pyridin-3-ylbenzyl)amino]phenoxy}quinolin-2(1H)-one; and
1-[2-(dimethylamino)ethyl]-3-{3′-[(2-oxo-1,2-dihydroquinolin-4-yl)oxy]biphenyl-4-yl}urea;
or a pharmaceutically acceptable salt thereof.

17. A composition comprising a compound of claim 1 and a pharmaceutically acceptable carrier.

18. The composition of claim 17, wherein the pharmaceutically acceptable carrier suitable for oral administration and the composition comprises an oral dosage form.

19. A composition comprising a compound of claim 1; a second compound selected from the group consisting of a topoisomerase I inhibitor, a MEK 1/2 inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab and lavendustin A; and a pharmaceutically acceptable carrier.

20. The composition of claim 19, wherein the second compound is Avastin.

21. A method of treating a PI3K-related disorder, comprising administering to a mammal in need thereof a compound of claim 1 in an amount effective to treat a PI3K-related disorder.

22. The method of claim 21, wherein the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

23. The method of claim 22, wherein the PI3K-related disorder is cancer.

24. The method of claim 23, wherein the cancer is selected from the group consisting of leukemia, skin cancer, bladder cancer, breast cancer, uterus cancer, ovarian cancer, prostate cancer, non-small cell lung cancer, colon cancer, pancreas cancer, renal cancer, gastric cancer, and brain cancer.

25. A method of treating an mTOR-related disorder, comprising administering to a mammal in need thereof a compound of claim 1 in an amount effective to treat an mTOR-related disorder.

26. The method of claim 25, wherein the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

27. The method of claim 26, wherein the mTOR-related disorder is cancer.

28. The method of claim 27, wherein the cancer is selected from the group consisting of leukemia, skin cancer, bladder cancer, breast cancer, uterus cancer, ovarian cancer, prostate cancer, non-small cell lung cancer, colon cancer, pancreas cancer, renal cancer, gastric cancer, and brain cancer.

29. A method of treating advanced renal cell carcinoma, comprising administering to a mammal in need thereof a compound of claim 1 in an amount effective to treat advanced renal cell carcinoma.

30. A method of treating acute lymphoblastic leukemia, comprising administering to a mammal in need thereof a compound of claim 1 in an amount effective to treat acute lymphoblastic leukemia.

31. A method of treating acute malignant melanoma, comprising administering to a mammal in need thereof a compound of claim 1 in an amount effective to treat malignant melanoma.

32. A method of treating soft-tissue or bone sarcoma, comprising administering to a mammal in need thereof a compound of claim 1 in an amount effective to treat soft-tissue or bone sarcoma.

33. A method of treating a cancer selected from the group consisting of leukemia, skin cancer, bladder cancer, breast cancer, uterus cancer, ovarian cancer, prostate cancer, non-small cell lung cancer, colon cancer, pancreas cancer, renal cancer, gastric cancer, and brain cancer comprising administering to a mammal in need thereof the composition of claim 20 in an amount effective to treat the cancer.

34. A method of inhibiting mTOR in a subject, comprising administering to a subject in need thereof a compound of claim 1 in an amount effective to inhibit mTOR.

35. A method of inhibiting PI3K in a subject, comprising administering to a subject in need thereof a compound of claim 1 in an amount effective to inhibit PI3K.

36. A method of inhibiting mTOR and PI3K together in a subject, comprising administering to a subject in need thereof a compound of claim 1 in an amount effective to inhibit mTOR and PI3K.

37. A method of synthesizing a compound of claim 1 comprising treating a compound of the formula II: 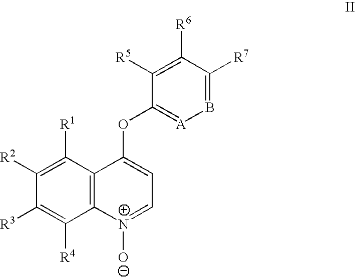
with a compound selected from tosyl chloride or benzoyl chloride, followed by hydrolysis with a base, wherein A, B, R¹, R², R³, R⁴, R⁵, R⁶ and R⁷ are defined as in claim 1.

38. The method of claim 37 wherein the base is selected from the group consisting of sodium carbonate, potassium carbonate, potassium hydroxide, sodium hydroxide, and triethylamine.

39. The method of claim 37 wherein the compound of formula II is made by reacting a compound of the formula: 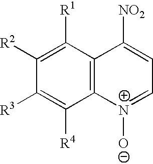
with a compound of the formula: 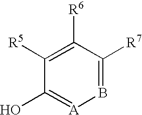
under basic conditions.

40. A method for preparing a compound according to claim 7, the method comprising replacing the iodine atom in a compound of formula III: 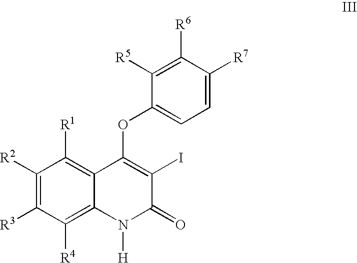
with a hydrogen atom.

41. The method of claim 40 wherein the iodine atom is replaced with a hydrogen atom by treating the compound of formula III with Zn and acetic acid.

42. The method of claim 40 wherein the compound of formula III is prepared by a process comprising reacting a compound of formula: 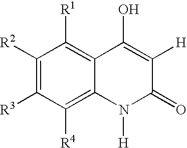
with a compound of formula: 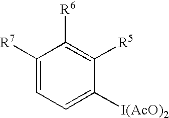
under basic conditions.

43. The method according to claim 42, wherein R¹ and R⁴ are both H.
